(19) United States
(12) Patent Application Publication
(10) Pub. No.: US 2014/0343050 A1
(43) Pub. Date: Nov. 20, 2014

(54) PRODRUGS OF D-ISOGLUTAMYL-[D/L]-TRYPTOPHAN

(71) Applicant: APTOEX TECHNOLOGIES, INC., TORONTO (CA)

(72) Inventors: Tim Fat Tam, Vaughan (CA); Regis Leung-Toung, Mississauga (CA); Yingsheng Wang, Toronto (CA); Yanqing Zhao, Richmond Hill (CA); Tao Xin, Vaughan (CA); Wanren Li, Toronto (CA); Jolanta Maria Wodzinska, Mississauga (CA); Vrajlal S. Rabadia, Mississauga (CA); Christopher John Feeney, Toronto (CA)

(73) Assignee: APTOEX TECHNOLOGIES, INC., TORONTO, ON (CA)

(21) Appl. No.: 14/008,902

(22) PCT Filed: Mar. 30, 2012
(86) PCT No.: PCT/CA12/00304

§ 371 Date: Aug. 8, 2014

Related U.S. Application Data
(60) Provisional application No. 61/470,467, filed on Mar. 31, 2011.

(51) Int. Cl. C07D 209/20

(52) U.S. Cl. CPC C07D 209/20
USPC 514/232.5; 548/495; 514/419; 544/143; 514/235.2; 544/82

(57) ABSTRACT

Provided are carboxylic ester derivatives of formula (I), methods of preparing them, and methods for using them. These compounds are prodrugs of D-isoglutamyl-[D/L]-tryptophan. The in vitro bioconversion of some of the prodrugs to the parent drug D-isoglutamyl-D-tryptophan (or thymodepressin) was tested in human hepatocytes and in human blood. In vivo pharmacokinetic studies following oral administration of some of the prodrugs to rats are also reported.

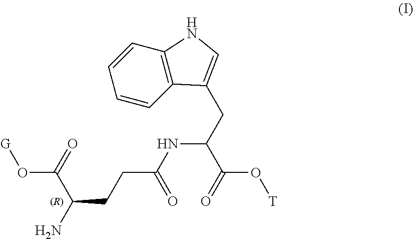

### TECHNICAL FIELD

This invention relates to the field of pharmaceutical sciences and more particularly to prodrugs of D-isoglutamyl-D-tryptophan and prodrugs of D-isoglutamyl-L-tryptophan.

### BACKGROUND

A prodrug is a compound that is modified in the body after its administration to provide an active drug. Depending on the therapeutic use and mode of administration, a prodrug may be used orally, for injection, intranasally, or in an inhaler formulation directed at lung tissues (Rautio et al. Nature Reviews Drug Discovery 7, 255-270 (February 2008). The use of prodrug compounds in an inhaler formulation directed at the lung tissue has been reviewed (Proceedings Of The American Thoracic Society Vol 1 2004, How the Lung Handles Drugs, Pharmacokinetics and Pharmacodynamics of Inhaled Corticosteroids, Julia Winkler, Guenther Hochhaus, and Hartmut Derendorf 356-363; H. Derendorf et al., Eur Respir J 2006; 28: 1042-1050).

For inhaler and intranasal means of administration, the minimization of oral bioavailability and systemic side effects by rapid clearance of absorbed active drug may be part of the design considerations. A prodrug designed for oral administration may prefer an improvement to oral bioavailability upon oral administration to animals, and appropriate chemical stability in simulated digestive fluids at pH 1.2 (also known as simulated gastric fluids) or pH 5.8 or 6.8 (also known as the simulated intestinal fluids). For prodrugs that are used in injection, the aqueous solubility of the compound is an important consideration.

The screening criteria for prodrugs depend on its mode of administration. However, a prodrug that can be readily hydrolyzed to the active drug in a human blood is a positive feature upon administration. Human blood has esterases that are capable of biotransforming some ester derivatives to the active drug (Derek Richter and Phyllis Godby Croft, Blood Esterases, Biochem J. 1942 December; 36(10-12): 746-757; Williams F M. Clinical significance of esterases in man. Clin Pharmacokinet. 1985 September-October; 10(5):392-403). In addition, prodrugs can be bioconverted in a human liver to the active drug (Baba et al., The pharmacokinetics of enalapril in patients with compensated liver cirrhosis Br J Clin Pharmacol. 1990 June; 29(6):766-9). Thus, regardless of the mode of administration, human hepatocyte and blood biotransformation results may be used to evaluate ester prodrugs.

D-Isoglutamyl-D-tryptophan (also known as H-D-Glu(D-Trp-OH)—OH or Apo805) is a synthetic hemoregulatory dipeptide developed for the treatment of autoimmune diseases including psoriasis (Sapuntsova, S. G., et al. (May 2002), Bulletin of Experimental Biology and Medicine, 133(5), 488-490). The sodium salt of H-D-Glu(D-Trp-OH)—OH (thymodepressin) is considered an effective treatment for psoriasis (U.S. Pat. No. 5,736,519), and is available as an injection ampoule in Russia.

D-Isoglutamyl-L-tryptophan (also known as H-D-Glu(L-Trp-OH)—OH or SCV-07) is reported as useful for modulating the immune system of a patient (U.S. Pat. No. 5,744,452), and useful for treating lung cancer (WO 2009/025830A1), tuberculosis (WO 2003/013572 A1), genital viral infections (WO 2006/076169), melanoma (WO 2007/123847), hemorrhagic viral infections (WO 2006/047702), respiratory viral infections (WO 2005/112639), hepatitis C (WO 2010/017178), and injury or damage due to disease of mucosa (WO 2008/100458). SCV-07 is also reported as a vaccine enhancer (WO 2006/116053).

### SUMMARY

The present invention is based, in part, on the elucidation of prodrugs of D-isoglutamyl-D-tryptophan (H-D-Glu(D-Trp-OH)—OH) and prodrugs of D-isoglutamyl-L-tryptophan (H-D-Glu(L-Trp-OH)—OH).

Illustrative embodiments of the present invention provide a compound of Formula I:

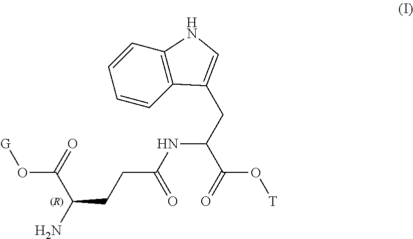

or a pharmaceutically acceptable salt thereof, wherein G is selected from the group consisting of: H, 2-morpholinoethyl, (CH₂)ₙCF₃, C₁-C₈ alkyl, benzyl and A₅-A₁₀ aryl; T is selected from the group consisting of: H, C₁-C₈ alkyl, 2-morpholinoethyl, (CH₂)ₙCF₃, CH₂CONR⁴R⁵, CH₂CH₂NR⁴R⁵, C₃-C₆ cycloalkyl, A₅-A₁₀ aryl,

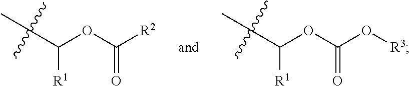

n is 1, 2, 3 or 4; R¹ is H or C₁-C₈ alkyl; R² is C₁-C₈ alkyl, C₃-C₆ cycloalkyl, or phenyl; R³ is C₁-C₈ alkyl, C₃-C₆ cycloalkyl, or phenyl; and R⁴ and R⁵ are either separate groups or together form a single group with the N to which they are bonded; when R⁴ and R⁵ are separate groups, R⁴ and R⁵ are independently selected from the group consisting of: C₁-C₆ alkyl; when R⁴ and R⁵ together with the N to which they are bonded form the single group, the single group is selected from the group consisting of: morpholinyl, N—(C₁-C₄ alkyl)-piperazinyl and piperidinyl; provided that if T is H, then G is 2-morpholinoethyl, (CH₂)ₙCF₃, C₁-C₈ alkyl or benzyl; if T is CH₂CONR⁴R⁵, CH₂CH₂NR⁴R⁵, or C₃-C₆ cycloalkyl, then G is H; and if T is C₁-C₈ alkyl, then G is 2-morpholinoethyl, (CH₂)ₙCF₃, or A₅-A₁₀ aryl.

Illustrative embodiments of the present invention provide a compound described herein wherein if G is H, then T is selected from the group consisting of: 2-morpholinoethyl, (CH₂)ₙCF₃, CH₂CONR⁴R⁵, CH₂CH₂NR⁴R⁵, C₃-C₆ cycloalkyl,

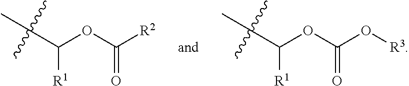

Illustrative embodiments of the present invention provide a compound described herein wherein if G is H, then T is selected from the group consisting of: 2-morpholinoethyl, (CH₂)ₙCF₃, CH₂CH₂NR⁴R⁵, C₃-C₆ cycloalkyl,

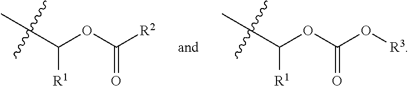

Illustrative embodiments of the present invention provide a compound described herein wherein if G is H, then T is selected from the group consisting of: 2-morpholinoethyl, (CH₂)ₙCF₃, CH₂CH₂NR⁴R⁵,

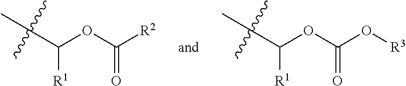

Illustrative embodiments of the present invention provide a compound described herein wherein a chiral carbon of a tryptophan moiety is in the D-configuration.

Illustrative embodiments of the present invention provide a compound described herein wherein a chiral carbon of a tryptophan moiety is in the L-configuration.

Illustrative embodiments of the present invention provide a compound described herein wherein G is H and T is A₅ to A₁₀ aryl.

Illustrative embodiments of the present invention provide a compound described herein wherein T is

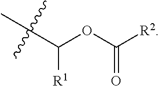

Illustrative embodiments of the present invention provide a compound described herein wherein T is

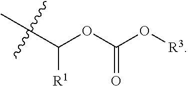

Illustrative embodiments of the present invention provide a compound described herein wherein T is (CH₂)ₙCF₃.

Illustrative embodiments of the present invention provide a compound described herein wherein T is 2-morpholinoethyl.

Illustrative embodiments of the present invention provide a compound described herein wherein G is 2-morpholinoethyl, (CH₂)ₙCF₃, or C₁-C₈ alkyl; and T is 2-morpholinoethyl, (CH₂)ₙCF₃, A₅ to A₁₀ aryl,

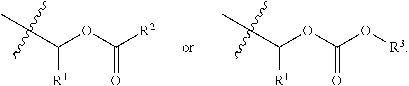

Illustrative embodiments of the present invention provide a compound described herein wherein T is C₁-C₈ alkyl.

Illustrative embodiments of the present invention provide a compound described herein wherein G is A₅ to A₁₀ aryl.

Illustrative embodiments of the present invention provide a compound described herein wherein T is isoamyl, G is indanyl.

Illustrative embodiments of the present invention provide a compound described herein wherein T is H.

Illustrative embodiments of the present invention provide a compound described herein wherein G is H.

Illustrative embodiments of the present invention provide a compound described herein wherein T is H and G is ethyl.

Illustrative embodiments of the present invention provide a compound described herein wherein T is H and G is benzyl.

Illustrative embodiments of the present invention provide a compound described herein wherein T is H and G is methyl.

Illustrative embodiments of the present invention provide a compound described herein wherein T is H and G is isoamyl.

Illustrative embodiments of the present invention provide a compound described herein wherein T is H and G is isopropyl.

Illustrative embodiments of the present invention provide a compound described herein wherein T is H, G is (CH₂)ₙCF₃ and n is 1.

Illustrative embodiments of the present invention provide a compound described herein wherein T is H, G is (CH₂)ₙCF₃ and n is 2.

Illustrative embodiments of the present invention provide a compound described herein T is H and G is 2-morpholinoethyl.

Illustrative embodiments of the present invention provide a compound described herein wherein T is

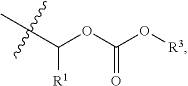

R¹ is methyl, R³ is cyclohexyl and G is H.

Illustrative embodiments of the present invention provide a compound described herein wherein T is 2-morpholinoethyl and G is H.

Illustrative embodiments of the present invention provide a compound described herein wherein T is cyclohexyl and G is H.

Illustrative embodiments of the present invention provide a compound described herein wherein T is

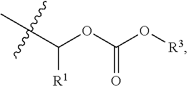

R¹ is methyl, R³ is cyclohexyl and G is H.

Illustrative embodiments of the present invention provide a compound described herein wherein T is (CH₂)ₙCF₃, n is 2 and G is H.

Illustrative embodiments of the present invention provide a compound described herein wherein T is

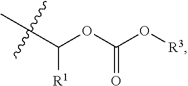

R¹ is methyl, R³ is ethyl and G is H.

Illustrative embodiments of the present invention provide a compound described herein wherein T is

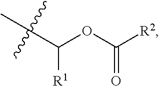

R¹ is H, R² is pent-2-yl and G is H.

Illustrative embodiments of the present invention provide a compound described herein wherein T is

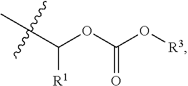

R¹ is methyl, R³ is isopropyl and G is H.

Illustrative embodiments of the present invention provide a compound described herein wherein T is CH₂CONR⁴R⁵, R⁴ is CH₃, R⁵ is CH₃ and G is H.

Illustrative embodiments of the present invention provide a compound described herein wherein T is CH₂CONR⁴R⁵, R⁴ is CH₃, R⁵ is CH₃ and G is H.

Illustrative embodiments of the present invention provide a compound described herein wherein T is

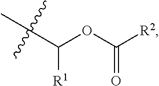

### R¹ is H, R² is C(CH₃)₂—CH₂CH₂CH₃ and G is H.

Illustrative embodiments of the present invention provide a compound described herein wherein T is (CH₂)ₙCF₃, n is 1 and G is H.

Illustrative embodiments of the present invention provide a compound described herein wherein T is (CH₂)ₙCF₃, n is 1 and G is H.

Illustrative embodiments of the present invention provide a compound described herein wherein T is indanyl and G is H.

Illustrative embodiments of the present invention provide a compound described herein wherein T is 2-methoxyphenyl and G is H.

Illustrative embodiments of the present invention provide a compound described herein wherein T is

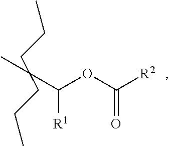

R¹ is H, R² is t-butyl and G is H.

Illustrative embodiments of the present invention provide a compound described herein wherein T is

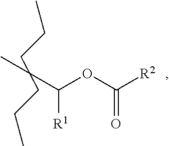

R¹ is H, R² is phenyl and G is H.

Illustrative embodiments of the present invention provide a compound described herein wherein T is (CH₂)ₙCF₃, n is 2, G is (CH₂)ₙCF₃ and n is 2.

Illustrative embodiments of the present invention provide a compound described herein wherein T is 2-morpholinoethyl and G is ethyl.

Illustrative embodiments of the present invention provide a compound described herein wherein T is

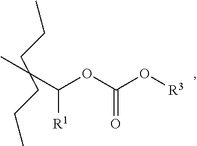

R¹ is methyl, R³ is ethyl and G is ethyl.

Illustrative embodiments of the present invention provide a compound described herein wherein T is 2-morpholinoethyl and G is 2-morpholinoethyl.

Illustrative embodiments of the present invention provide a compound described herein wherein T is benzyl and G is 2-morpholinoethyl.

Illustrative embodiments of the present invention provide a compound described herein wherein T is indanyl and G is 2-morpholinoethyl.

Illustrative embodiments of the present invention provide a compound described herein wherein T is 2-morpholinoethyl. G is (CH₂)ₙCF₃ and n is 2.

Illustrative embodiments of the present invention provide a compound described herein wherein T is 2-morpholinoethyl and G is isoamyl.

Illustrative embodiments of the present invention provide a compound described herein wherein T is (CH₂)ₙCF₃, n is 1, G is (CH₂)ₙCF₃ and n is 1.

Illustrative embodiments of the present invention provide a pharmaceutical formulation comprising a compound described herein and a pharmaceutically acceptable excipient.

Illustrative embodiments of the present invention provide a pharmaceutical composition described herein wherein the formulation is adapted for inhalation.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

FIG. 1 shows the average (n=5) concentration of H-D-Glu(D-Trp-OH)—OH (Apo805) in plasma after oral dosing of H-D-Glu(D-Trp-O—CH₂—O—CO-t-Bu)-OH (Apo839) and H-D-Glu(D-Trp-OH)—OH monopotassium salt (Apo805K1) (5 mg/kg) to rats demonstrating similar oral bioavailability of the prodrug.

FIG. 2 shows the average (n=5) concentration of H-D-Glu(D-Trp-OH)—OH (Apo805) in plasma after oral dosing of H-D-Glu(D-Trp-O—CH(CH₃)—O—CO—O-cyclohexyl)-OH (Apo843) and H-D-Glu(D-Trp-OH)—OH monopotassium salt (Apo805K1) (5 mg/kg) to rats demonstrating reduced oral bioavailability of the prodrug. The minimization of oral bioavailability is one feature to be considered for prodrugs designed for an inhaler mode of administration.

### DETAILED DESCRIPTION

The present invention is based, in part, on the elucidation of prodrugs of D-isoglutamyl-D-tryptophan and prodrugs of D-isoglutamyl-L-tryptophan.

As used herein, the symbol

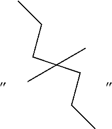

indicates the point at which the displayed moiety is attached to the remainder of the molecule. For example, pentyl or CH₃—CH₂—CH₂— may be shown as

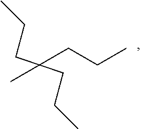

Another example is CH₃—CH₂—CH—CH₂—CH₃ (a pent-3-yl moiety) may be shown as

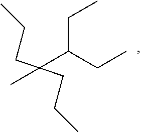

As used herein, the term “alkyl” means a branched or unbranched saturated hydrocarbon chain. Non-limiting, illustrative examples of alkyl moieties include, methyl, ethyl, propyl, isopropyl, n-propyl, butyl, sec-butyl, isobutyl, n-pentyl, hexyl, octyl and the like. When the terminology “Cₓ-C_{y}”, where x and y are integers, is used with respect to alkyl moieties, the ‘C’ relates to the number of carbon atoms the alkyl moiety. For example, methyl may be described as a C₁ alkyl and isobutyl may be described as a C₄ alkyl. C₁-C₄ alkyl means methyl (a C₁ alkyl), ethyl (a C₂ alkyl), propyl or isopropyl (a C₃ alkyl), butyl or sec-butyl or isobutyl or tert-butyl (a C₄ alkyl). All specific integers and ranges of integers within each range are specifically disclosed by the broad range. For example, C₁-C₈, specifically includes the following: C₁, C₂, C₃, C₄, C₅, C₆, C₇, C₈, C₁-C₂, C₁-C₃, C₁-C₄, C₁-C₅, C₁-C₆, C₁-C₇, C₁-C₈, C₂-C₃, C₂-C₄, C₂-C₅, C₂-C₅, C₂-C₇, C₂-C₈, C₃-C₄, C₃-C₅, C₃-C₆, C₃-C₇, C₃-C₈, C₄-C₅, C₄-C₆, C₄-C₇, C₄-C₈, C₅-C₆, C₅-C₇, C₅-C₈, C₆-C₇, C₆-C₈, and C₇-C₈. Another example is C₅-C₈ specifically includes C₅, C₆, C₇, C₈, C₅-C₆, C₅-C₇, C₅-C₈, C₆-C₇, C₆-C₈, and C₇-C₈.

As used herein the term “aryl” means any moiety which has at least a portion of the moiety that conforms to Hückel's rule. This includes moieties that are hydrocarbons and moieties that include heteroatoms. For clarity, an aryl moiety as a whole does not need to conform to Hückel's rule as long as some portion of the aryl moiety, when considered in the absence of the remainder of the moiety, does conform to Hackers rule. Non-limiting, illustrative examples of aryl moieties include phenyl, benzyl, indanyl, 2-methoxyphenyl, 3-methoxyphenyl and 2-fluorophenyl. When the terminology “Aₓ-A_{y}”, where x and y are integers, is used with respect to aryl moieties, the ‘A’ relates to the total number of carbon and heteroatoms in the aryl moiety. For example, 1-fluorophenyl may be described as an A₇ aryl group and 2-methoxyphenyl may be described as an A₈ aryl group. Furan is an example of an A₅ aryl group. All specific integers and ranges of integers within each range are specifically disclosed by the broad range. For example, A₅-A₁₀, specifically includes the following: A₅, A₆, A₇, A₈, A₉, A₁₀, A₅-A₆, A₅-A₇, A₅-A₈, A₅-A₉, A₅-A₁₀, A₆-A₇, A₆-A₈, A₆-A₉, A₆-A₁₀, A₇-A₈, A₇-A₈, A₇-A₁₀, A₈-A₉, A₈-A₁₀ and A₉-A₁₀.

As used herein, the term “mofetil” means a morpholinoethyl radical having the structure:

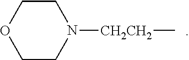

Mofetil is often referred to by the IUPAC name 2-morpholinoethyl.

The following acronyms and/or shorthand notation are also used herein.

| Acronym and/or Shorthand | Explanation of Acronym and/or Shorthand |
|---|---|
| EDCl | 1-ethyl-3-(3-dimethylaminopropyl) |
| | carbodiimide) hydrochloride |
| DIPEA | diisopropylethylamine |
| DMF | dimethylformamide |
| DMSO | dimethylsulfoxide |
| RT | room temperature |
| HOSu | hydroxysuccinimide |
| Boc-D-Glu(O-Bzl)-OH | 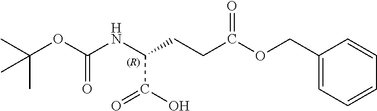 |
| Boc-D-Glu-O-isoamyl | 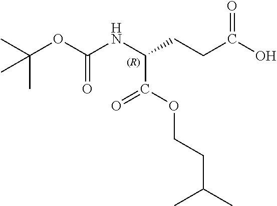 |
| Boc-D-Glu-OBzl | 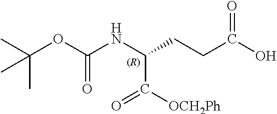 |
| Boc-D-Glu(O-Bzl)-O-isoamyl | 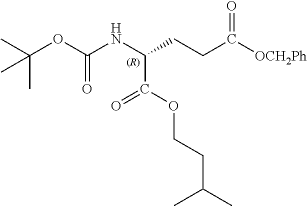 |
| Cbz-D-Glu-O-Bzl | 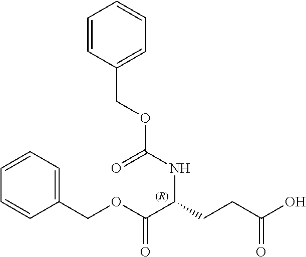 |
| H-D-Glu(D-Trp-OH)—OH | D-gamma-glutamyl-D-tryptophan |
| H-D-Glu(L-Trp-OH)—OH | D-gamma-glutamyl-L-tryptophan |
| H-D-Glu(Trp-OH)—OH | 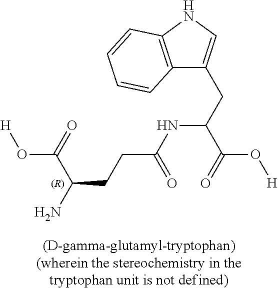 |
| Boc-D-Glu(D-Trp-O-Bzl)-O-isoamyl | 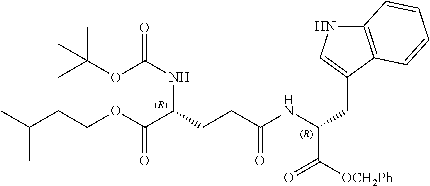 |
| Boc-D-Glu(D-Trp-OH)—O-isoamyl | 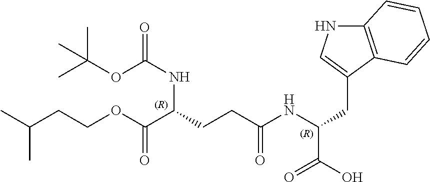 |
| H-D-Glu(D-Trp-OH)—O-isoamyl | 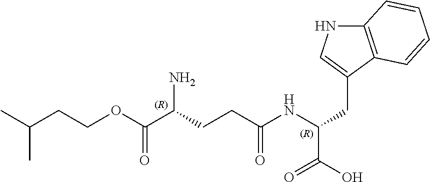 |
| Boc-D-Glu(D-Trp-O-Bzl)-OEt | 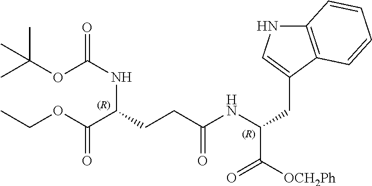 |
| Boc-D-Glu(D-Trp-OH)—OEt | 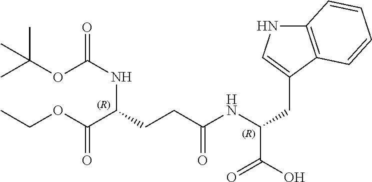 |
| H-D-Glu(D-Trp-OH)—OEt | 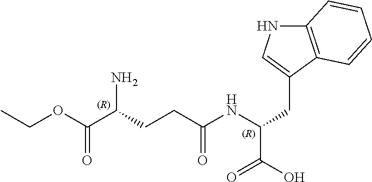 |
| Boc-D-Glu(D-Trp-O-Bzl)-O-iPr | 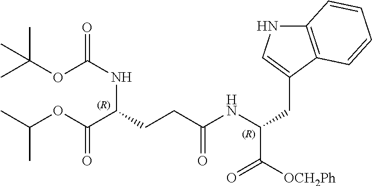 |
| Boc-D-Glu(D-Trp-OH)—O-iPr | 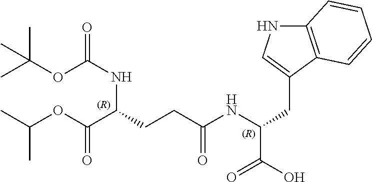 |
| H-D-Glu(D-Trp-OH)—O-iPr | 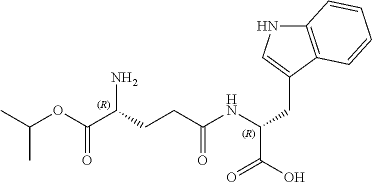 |
| Boc-D-Glu(D-Trp-OH)—OCH₂CH₂CF₃ | 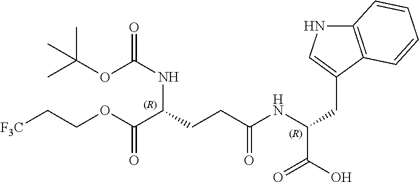 |
| H-D-Glu(D-Trp-OH)—OCH₂CH₂CF₃ | 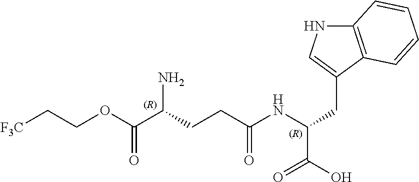 |
| Boc-D-Glu(D-Trp-OH)—O-Bzl | 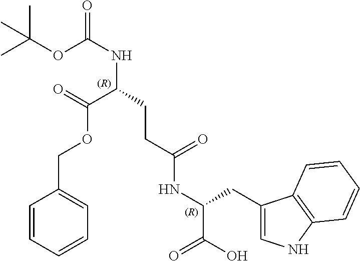 |
| H-D-Glu(D-Trp-OH)—O-Bzl | 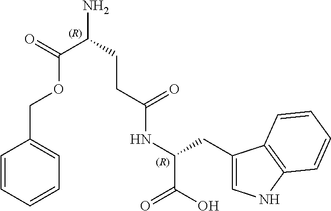 |
| H-D-Glu(D-Trp-OH)—OCH₂CF₃ | 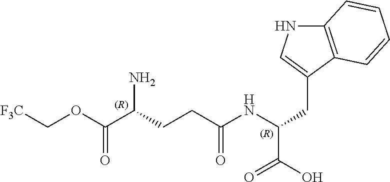 |
| Cbz-D-Glu(D-Trp-OH)—O-Bzl | 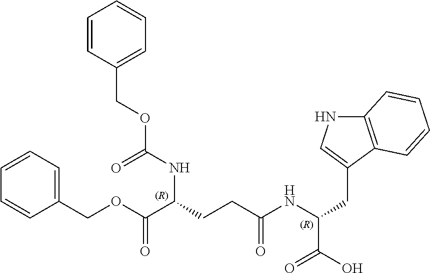 |
| Cbz-D-Glu(D-Trp- O—CH(CH₃)—O—CO—O-cyclohexyl)-O-Bzl | 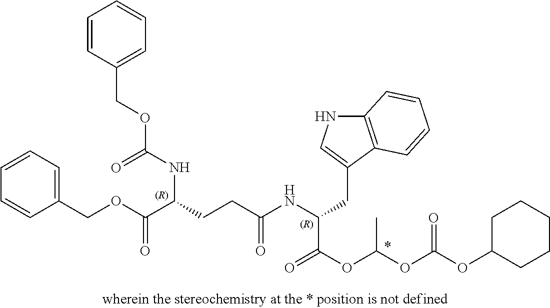 |
| H-D-Glu(D-Trp-O—CH(CH₃)—O—CO—O- cyclohexyl)-O | 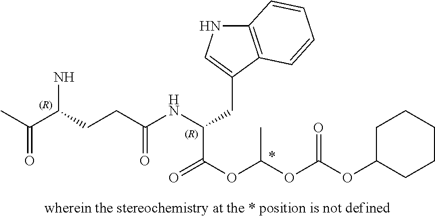 |
| Cbz-D-Glu(D-Trp- O—CH(CH₃)—O—CO—O—Et)—O-Bzl | 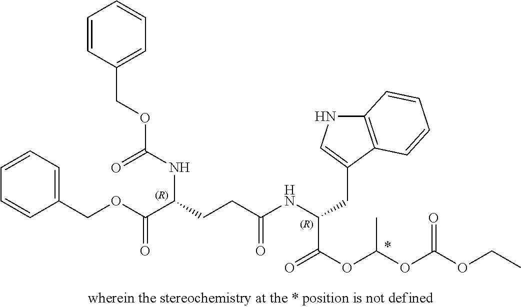 |
| H-D-Glu(D-Trp- O—CH(CH₃)—O—CO—O—Et)—OH | 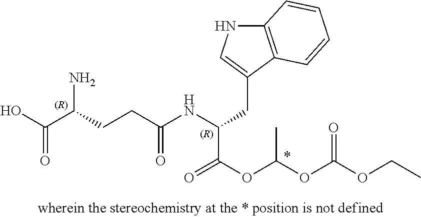 |
| Cbz-D-Glu(D-Trp- O—CH(CH₃)—O—CO—O—i-Pr)—O-Bzl | 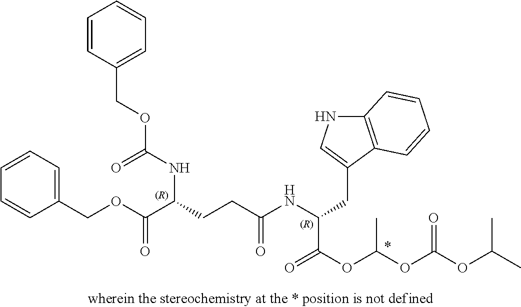 |
| H-D-Glu(D-Trp- O—CH(CH₃)—O—CO—Oi—-Pr)—OH | 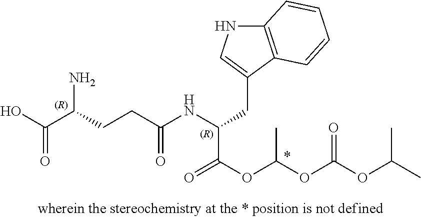 |
| Cbz-D-Glu(D-Trp- O—CH₂CO—N(CH₃)₂)—O-Bzl | 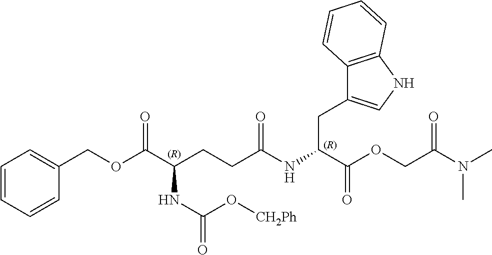 |
| H-D-Glu(D-Trp- O—CH₂CO—N(CH₃)₂)—OH | 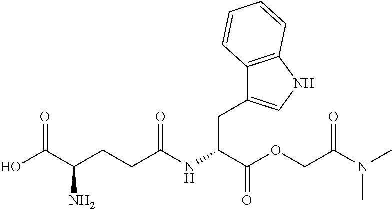 [SMILES: CN(C)C(=O)COC(=O)[C@@H](Cc1c[nH]c2ccccc12)NC(=O)CC[C@@H](N)C(=O)O] |
| Cbz-D-Glu(D-Trp-O-mofetil)-O-Bzl | 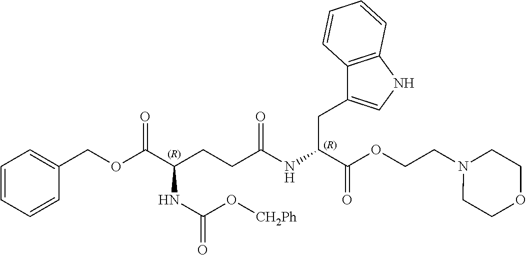 |
| H-D-Glu(D-Trp-O-mofetil)-OH | 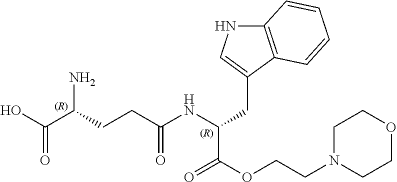 |
| Cbz-D-Glu(D-Trp-OCH₂O—CO—Ph)—O-Bzl | 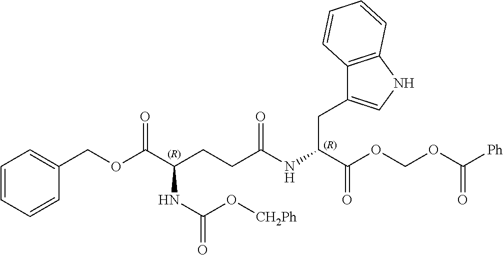 |
| H-D-Glu(D-Trp-OCH₂O—CO—Ph)—OH | 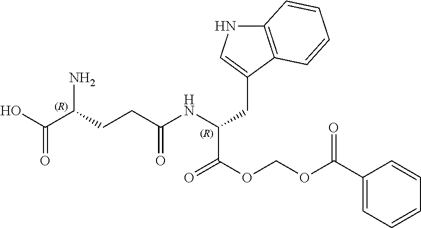 |
| H-D-Glu(D-Trp-OCH₂O—CO-[pent-3-yl])-OH | 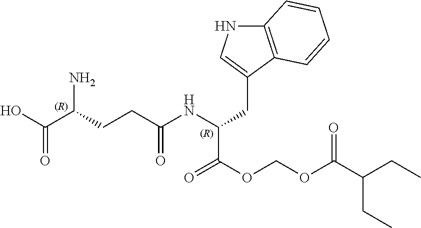 |
| H-D-Glu(D-Trp- OCH₂O—CO—C(CH₃)₂—CH₂CH₂CH₃)—OH | 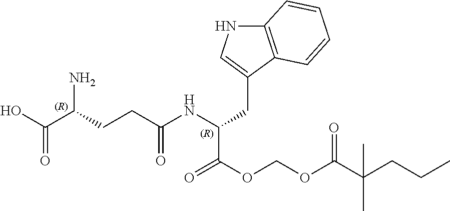 |
| H-D-Gru(D-Trp-OCH₂CH₂CF₃)—OH | 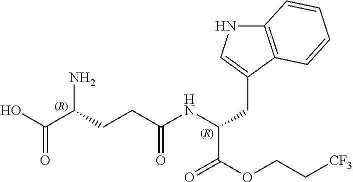 |
| Boc-D-Glu(D-Trp-O-mofetil)-O-mofetil | 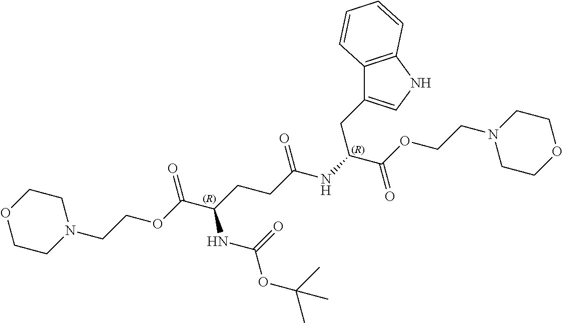 |
| H-D-Glu(D-Trp-O-mofetil)-O-mofetil | 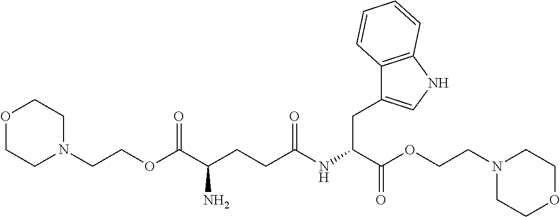 [SMILES: N[C@H](CCC(=O)N[C@H](Cc1c[nH]c2ccccc12)C(=O)OCCN1CCOCC1)C(=O)OCCN1CCOCC1] |
| H-D-Glu(D-Trp- O—CH₂CH₂CF₃)—O—CH₂CH₂CF₃ | 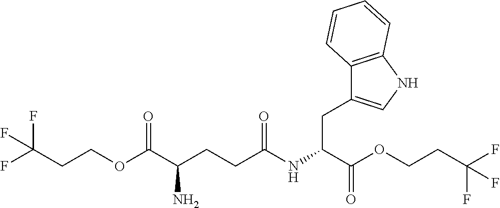 [SMILES: N[C@H](CCC(=O)N[C@H](Cc1c[nH]c2ccccc12)C(=O)OCCC(F)(F)F)C(=O)OCCC(F)(F)F] |
| Cbz-D-Glu(D-Trp-OH)—OEt | 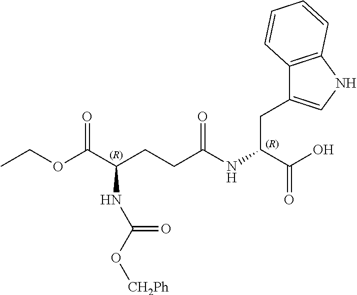 |
| Cbz-D-Glu(D-Trp- O—CH(CH₃)—O—CO—O-cyclohexyl)-OEt | 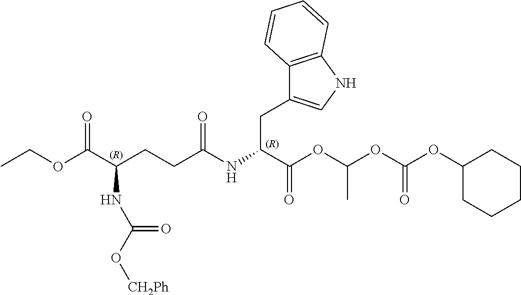 |
| H-D-Glu(D-Trp- O—CH(CH₃)—O—CO—O-cyclohexyl)-OEt | 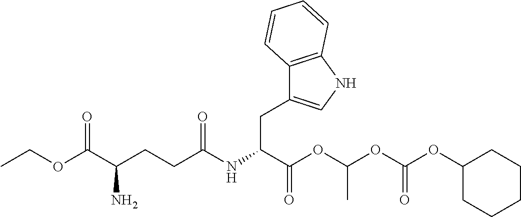 [SMILES: CCOC(=O)[C@H](N)CCC(=O)N[C@H](Cc1c[nH]c2ccccc12)C(=O)OC(C)OC(=O)OC1CCCCC1] |
| Cbz-D-Glu(D-Trp- O—CH(CH₃)—O—CO—OEt)—OEt | 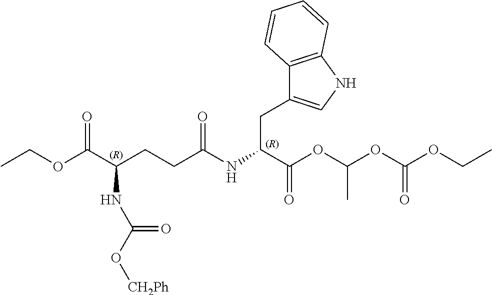 |
| H-D-Glu(D- Trp-O—CH(CH₃)—O—COO—Et)—OEt | 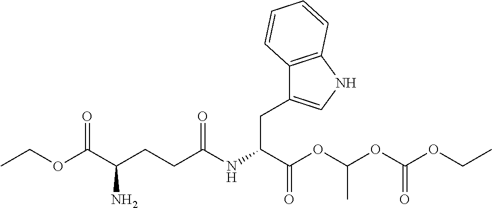 [SMILES: CCOC(=O)OC(C)OC(=O)[C@@H](Cc1c[nH]c2ccccc12)NC(=O)CC[C@@H](N)C(=O)OCC] |
| Cbz-D-Glu(D-Trp-O-mofetil)-OEt | 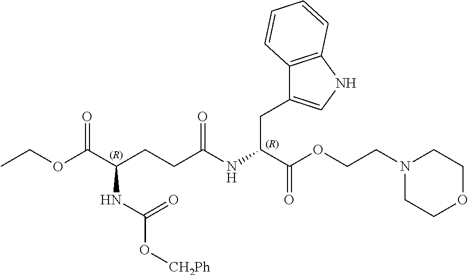 |
| H-D-Glu(D-Trp-O-mofetil)-OEt | 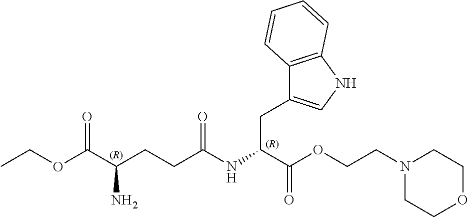 |
| H-D-Glu(D-Trp-O-mofetil)-O—CH₂CH₂CF₃ | 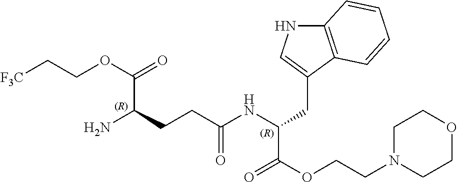 |
| H-D-Glu(D-Trp-O-mofetil)-O-isoamyl | 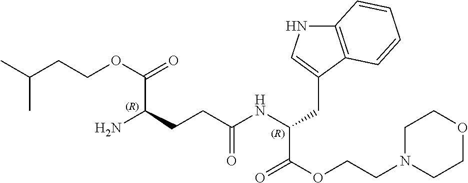 |
| H-D-Glu(D-Trp-O-5-indanyl)-O-mofetil | 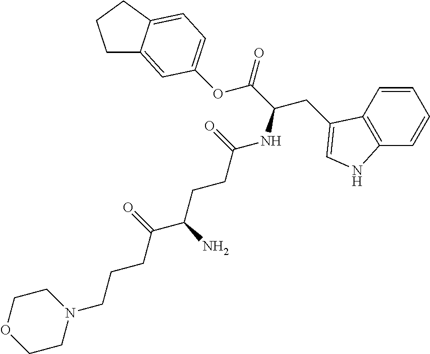 [SMILES: N[C@H](CCC(=O)N[C@H](Cc1c[nH]c2ccccc12)C(=O)Oc1ccc2c(c1)CCC2)C(=O)CCCN1CCOCC1] |
| H-D-Glu(D-Trp-OH)-O-mofetil | 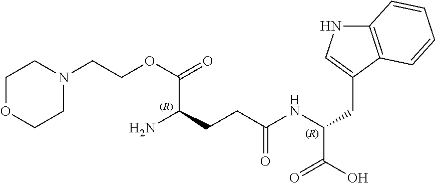 |
| H-D-Glu(D-Trp-O-mofetil)-O-Bzl | 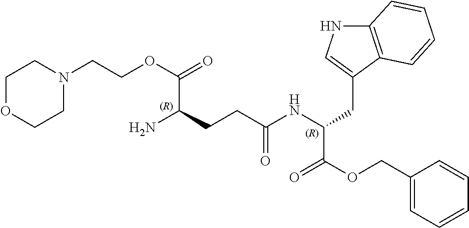 |
| H-D-Glu(D-Trp-OCH₂O—CO—C(CH₃)₃)—OH | 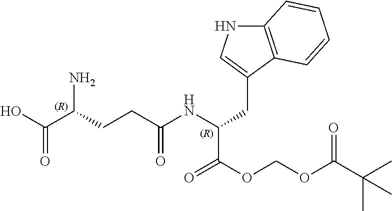 |

Compounds of the present invention may be described by Formula I:

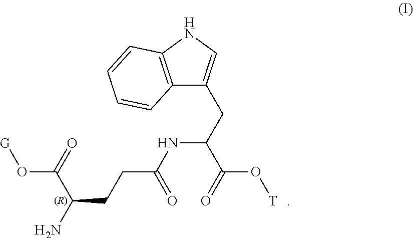

In Formula I:
G is selected from the group consisting of: H, 2-morpholinoethyl, (CH₂)ₙCF₃, C₁-C₈ alkyl, and A₅-A₁₀ aryl; and
T is selected from the group consisting of: H, C₁-C₈ alkyl, 2-morpholinoethyl, (CH₂)ₙCF₃, CH₂CONR⁴R⁵, CH₂CH₂NR⁴R⁵, C₃-C₆ cycloalkyl, A₅-A₁₀ aryl,

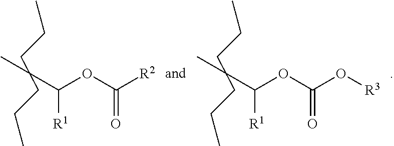

In the (CH₂)ₙCF₃ moiety, n is 1, 2, 3 or 4.

In those moieties in which R¹ appears, R¹ is H or C₁-C₃ alkyl.

In the moiety in which R² appears, R² is C₁-C₈ alkyl, C₃-C₆ cycloalkyl, or phenyl.

In the moiety in which R³ appears, R³ is C₁-C₈ alkyl, C₃-C₆ cycloalkyl, or phenyl.

In those moieties in which R⁴ and R⁵ appear, R⁴ and R⁵ are either separate groups or together form a single group with the N to which they are bonded. When R⁴ and R⁵ are separate groups, R⁴ and R⁵ are independently selected from the group consisting of: C₁-C₆ alkyl. When R⁴ and R⁵ together with the N to which they are bonded form the single group, the single group is selected from the group consisting of: morpholinyl, N—(C₁-C₄ alkyl)-piperazinyl and piperidinyl.

Compounds of Formula I are limited to compounds in which if T is H, then G is 2-morpholinoethyl, (CH₂)ₙCF₃, C₁-C₈ alkyl or benzyl (benzyl is a particular A₅-A₁₀ aryl) and if T is CH₂CONR⁴R⁵, CH₂CH₂NR⁴R⁵, or C₃-C₆ cycloalkyl, then G is H and if T is C₁-C₈ alkyl, G is 2-morpholinoethyl, (CH₂)ₙCF₃, or A₅-A₁₀ aryl.

In particular embodiments, compounds of Formula I may be further limited to compounds in which when G is H, T is selected from the group consisting of: 2-morpholinoethyl, (CH₂)ₙCF₃, CH₂CONR⁴R⁵, CH₂CH₂NR⁴R⁵, C₃-C₆ cycloalkyl,

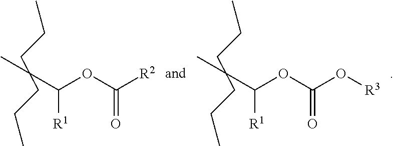

In particular embodiments, compounds of Formula I may be further limited to compounds in which when G is H, T is selected from the group consisting of: 2-morpholinoethyl, (CH₂)ₙCF₃, CH₂CH₂NR⁴R⁵, C₃-C₆ cycloalkyl,

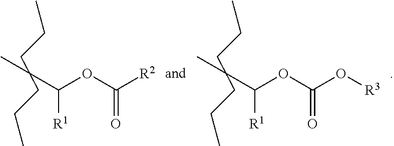

In particular embodiments, compounds of Formula I may be further limited to compounds in which when G is H, T is selected from the group consisting of: 2-morpholinoethyl, (CH₂)ₙCF₃, CH₂CH₂NR⁴R⁵,

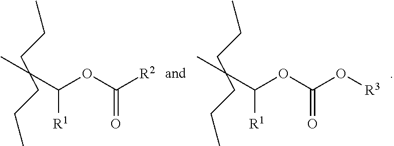

In particular embodiments, compounds of Formula I specifically exclude compounds in which T is A₅-A₁₀ aryl and G is H.

In particular embodiments, compounds of Formula I specifically exclude compounds in which G is C₁-C₈ alkyl and T is H.

In particular embodiments, compounds of Formula I specifically exclude compound in which T is H and G is H.

In particular embodiments, compounds of Formula I specifically exclude compounds in which G is C₁-C₈ alkyl and T is C₁-C₈ alkyl.

In particular embodiments, compounds of Formula I are also compounds of Formula IA:

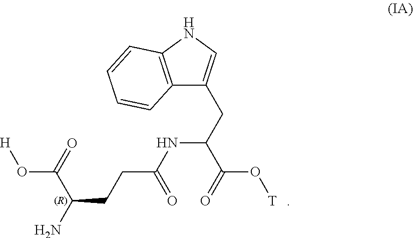

In Formula IA, T is selected from the group consisting of: 2-morpholinoethyl;

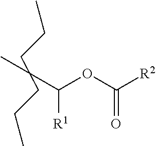

wherein R¹ is H or C₁-C₃ alkyl, and R² is C₁-C₈ alkyl, C₃-C₆ cycloalkyl, or phenyl;

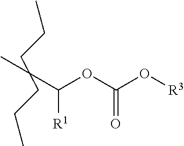

wherein R¹ is H or C₁-C₃ alkyl, and R³ is C₁-C₈ alkyl, phenyl, or C₃-C₈ cycloalkyl; and —(CH₂)ₙCF₃ wherein n is 1 to 4.

In particular embodiments, compounds of Formula I are also compounds of Formula IB:

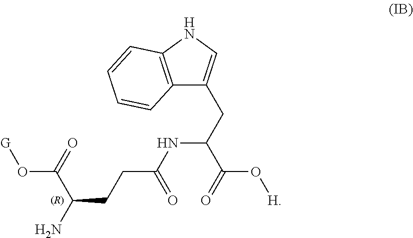

In Formula IB, G is selected the group consisting of: 2-morpholinoethyl; and (CH₂)ₙCF₃ wherein n is 1 to 4.

In particular embodiments, compounds of Formula I are also compounds of Formula IC:

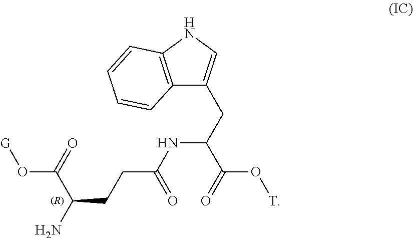

In Formula IC:
G is selected from the group consisting of: C₁-C₈ alkyl, 2-morpholinoethyl, —(CH₂)ₙCF₃ wherein n is 1 to 4, and A₅-A₁₀ aryl; and
T is selected from the group consisting of: 2-morpholinoethyl;

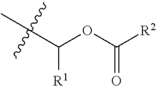

wherein R¹ is H or C₁-C₃ alkyl, and R² is C₁-C₈ alkyl, C₃-C₆ cycloalkyl, or phenyl;

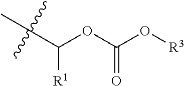

wherein R¹ is H or C₁-C₃ alkyl, and R³ is C₁-C₈ phenyl, or C₃-C₆ cycloalkyl; and —(CH₂)ₙCF₃ wherein n is 1 to 4.

Compounds of Formulas I, IA, IB and IC comprise a tryptophan moiety. The tryptophan moiety may be considered as the following moiety:

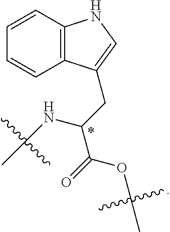 [SMILES: *NC(Cc1c[nH]c2ccccc12)C(=O)O*]

Of particular interest in the tryptophan moiety is a chiral carbon, which is denoted above by the “*”. The chiral carbon of the tryptophan moiety may be in either the L-configuration or the D-configuration. In some embodiments, the compounds of Formula I, IA, IB and/or IC comprise a chiral carbon of the tryptophan moiety in the D-configuration. In other embodiments, the compounds of Formula I, IA, IB and/or IC comprise a chiral carbon of the tryptophan moiety in the L-configuration. In still other embodiments, compositions of compounds comprising compounds of Formulas I, IA, IB and/or IC may comprise some compounds in which the chiral carbon of the tryptophan moiety is in the L-configuration and other compounds in which the chiral carbon of the tryptophan moiety is in the D-configuration.

Compounds of the present invention may also be provided in the form of a salt or a pharmaceutically acceptable salt. An example of a pharmaceutically acceptable salt of this invention is Apo900, H-D-Glu(D-Trp-O-mofetil)-O-Et.2HCl, (ethyl(2R)-2-amino-5-({(2R)-3-(1H-indol-3-yl)-1-[2-(morpholin-4-yl)ethoxy]-1-oxopropan-2-yl}amino)-5-oxopentanoate dihydrochloride), which may be diagrammatically represented by the following structure:

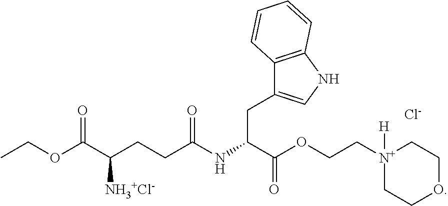

Compounds of the present invention may be pharmaceutically acceptable salts and include salts of acidic or basic groups present in compounds described herein. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Suitable base salts include, but are not limited to, aluminum, calcium, lithium, magnesium, potassium, sodium, zinc, and diethanolamine salts. For a review on pharmaceutically acceptable salts see Berge et al., 66 J. Pharm. Sci. 1-19 (1977).

Syntheses of compounds of Formula I are outlined below in Schemes 1, 2 and 3.

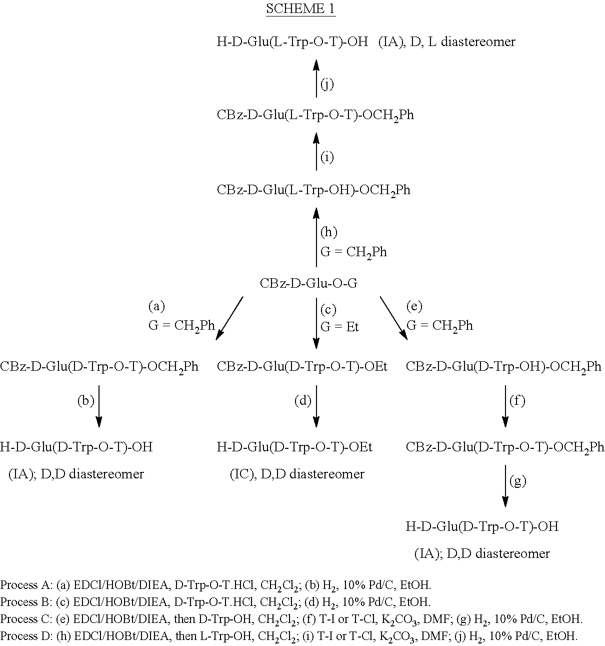

Process A describes synthesis of a compound of Formula IA wherein the dipeptide is H-D-Glu(D-Trp-O-T)-OH is used as an illustrative example. The process may be readily adapted to make other compounds of Formula I.

In process A step (a), Cbz-D-Glu-OCH₂Ph is coupled with D-Trp-O-T.HCl ester wherein T is C₃-C₆ cycloalkyl, or an A₅-A₁₀ aryl to give the compound Cbz-D-Glu(D-Trp-O-T)-OCH₂Ph using EDCl, HOBt, DIEA (diisopropylethylamine) in CH₂Cl₂. In step (b), hydrogenation of the Cbz-D-Glu(D-Trp-O-T)-OCH₂Ph give the compound of formula (1A) as shown above.

Process B describes synthesis of a compound of Formula IC wherein the dipeptide is H-D-Glu(D-Trp-O-T)-O-G is used as an illustrative example. The process may be readily adapted to make other compounds of Formula I.

In process B step (c), Cbz-D-Glu-OEt is coupled with D-Trp-O-T.HCl ester wherein T is C₃-C₆ cycloalkyl, or an A₅-A₁₀ aryl to give the compound Cbz-D-Glu(D-Trp-O-T)-OEt using EDCl, HOBt, DIEA in CH₂Cl₂. In step (d), hydrogenation of the Cbz-D-Glu(D-Trp-O-T)-OEt give the compound of formula (IC) wherein G is ethyl, T is C₃-C₆ cycloalkyl, or an A₅-A₁₀ aryl.

Process C describes synthesis of compounds of Formula IA wherein T is N-morpholinylethyl;

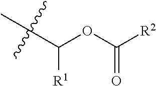

wherein R¹ is H or C₁-C₃ alkyl, and R² is C₁-C₈ alkyl, C₃-C₆ cycloalkyl, or phenyl;

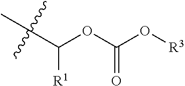

wherein R¹ is H or C₁-C₃ alkyl, and R³ is C₁-C₈ alkyl, phenyl, or C₃-C₆ cycloalkyl; or (CH₂)ₙCF₃ wherein n is 1 to 4. The process may be readily adapted to make other compounds of Formula I.

In process C step (e), Cbz-D-Glu-OCH₂Ph is coupled with D-Trp-OH to give the compound Cbz-D-Glu(D-Trp-OH)—OCH₂Ph using EDCl, HOBt, DIEA in CH₂Cl₂. In step (f), Cbz-D-Glu(D-Trp-OH)—OCH₂Ph is reacted with potassium carbonate and T-Cl or T-I wherein T is defined above under the compound of formula (IA) in process C to give the dipeptide Cbz-D-Glu(D-Trp-O-T)-OCH₂Ph. In step (g), hydrogenation of the Cbz-D-Glu(D-Trp-O-T)-OCH₂Ph gives the peptide H-D-Glu(D-Trp-O-T)-OH, a compound of formula (IA) wherein T is defined above under process C.

Process D describes synthesis of compounds of Formula IA wherein T is N-morpholinylethyl;

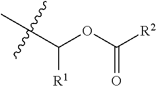

wherein R¹ is H or C₁-C₃ alkyl, and R² is C₁-C₈ alkyl, C₃-C₆ cycloalkyl, or phenyl;

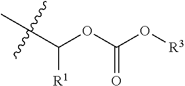

wherein R¹ is H or C₁-C₃ alkyl, and R³ is C₁-C₈ alkyl, phenyl, or C₃-C₆ cycloalkyl; or (CH₂)ₙCF₃ wherein n is 1 to 4. The process may be readily adapted to make other compounds of Formula I.

Process D is identical to process C, with the exception that L-Trp-OH is used instead of D-Trp-OH in the process. As an illustrative example, by replacing the D-Trp-OH in step (e) with L-Trp-OH, Apo894 (D,L), a compound of formula IA wherein T=CH₂CON(CH₃)₂ can be made. The procedure is further exemplified in a particular embodiment in Example 16.

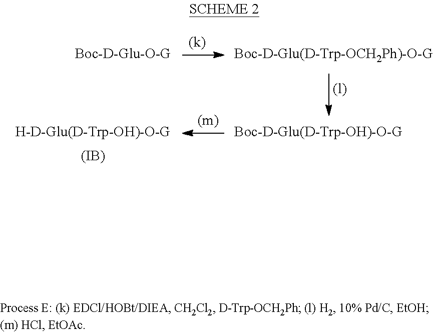

Process E describes synthesis of a compound of Formula IB. The process may be readily adapted to make other compounds of Formula I.

In process E step (k), Boc-D-Glu-O-G wherein G is C₁-C₈ alkyl, trifluoropropyl is coupled to the D-Trp-OCH₂Ph.HCl with EDCl/HOBt/DEIA in CH₂Cl₂ to give Boc-D-Glu(D-Trp-OCH₂Ph)-O-G. In step (I), hydrogenation over Pd/C in ethanol gives Boc-D-Glu(D-Trp-OH)—O-G. In step (m), de-Boc of Boc-D-Glu(D-Trp-OH)—O-G using HCl in EtOAc affords the compound of Formula (IB).

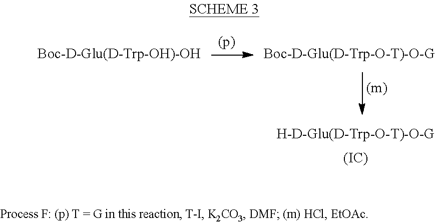

Process F describes synthesis of a compound wherein G=T=N-morpholinylethyl. The process may be readily adapted to make other compounds of Formula I.

In process F step (p), Boc-D-Glu(D-Trp-OH)—OH is reacted with T-I, K₂CO₃, DMF to give Boc-D-Glu(D-Trp-O-T)-O-G wherein G=T, and G is N-morpholinylethyl. In step (m), treatment of Boc-D-Glu(D-Trp-O-T)-O-G gives the compound (IC) wherein G=T.

In a similar manner, compounds of Formula I with the gamma-D-glutamyl and L-tryptophanyl moiety may be prepared using the information as described in processes A to F adapted to suit the particulars of the desired product.

Compounds of Formula I that exist in free base form may be converted to their pharmaceutically acceptable salts by treatment with the appropriate inorganic or organic acid. Salts of the compounds of Formula I may be converted to the free base form or to another salt.

Compounds of the present invention or salts thereof may be formulated into a pharmaceutical formulation. Many compounds of this invention are generally water soluble and may be formed as salts. In such cases, pharmaceutical compositions in accordance with this invention may comprise a salt of such a compound, preferably a physiologically acceptable salt, which are known in the art. Pharmaceutical preparations will typically comprise one or more carriers acceptable for the mode of administration of the preparation, be it by injection, inhalation, topical administration, lavage, or other modes suitable for the selected treatment. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as improved PASI score. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as a desirable PASI score. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

As used herein, a “subject” may be a human, non-human primate, rat, mouse, cow, horse, pig, sheep, goat, dog, cat, etc. The subject may be suspected of having or at risk for having psoriasis and/or atopic dermatitis and/or a medical condition wherein an agent is used in modulating the immune system. Diagnostic methods for psoriasis, atopic dermatitis and various disorders for which immune modulating compounds are used and the clinical delineation of those conditions' diagnoses are known to those of ordinary skill in the art.

### EXAMPLES

The following examples are illustrative of some of the embodiments of the invention described herein. These examples do not limit the spirit or scope of the invention in any way.

### Example 1

### Preparation of H-D-Glu(D-Trp-OH)—OCH₂CH₂CF₃, Apo878

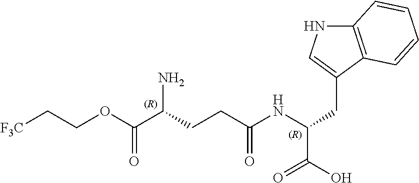

### A. Preparation of Boc-D-Glu(OH)—OCH₂CH₂CF₃

To a solution of Boc-D-Glu(O-Bzl)-OH (7.00 g, 20.7 mmol) in DMF (50 mL) was added N-hydroxysuccinimide (HOSu, 2.63 g, 22.8 mmol) followed by EDCl.HCl (4.38 g, 22.8 mmol). After stirring for 1 h, 3,3,3-trifluoropropan-1-ol (3.2 mL, 36.3 mmol) and DIPEA (4.0 mL, 22.8 mmol) were added and the resulting mixture was stirred at RT for overnight. The reaction mixture was quenched with de-ionized water and extracted with EtOAc. The organic fraction was washed with brine, dried over anhydrous Na₂SO₄, filtered and then evaporated to dryness under reduced pressure. A mixture of the crude Boc-D-Glu(O-Bzl)-OCH₂CH₂CF₃ and 1.6 g of wet 10% Pd—C in EtOH (100 mL) was then hydrogenated under 45 psi of hydrogen gas pressure in a Parr apparatus for 1.5 h. The mixture was filtered, and the filtrate was concentrated in vacuo to afford Boc-D-Glu(OH)—OCH₂CH₂CF₃ (crude yield=81%), which was used without further purification.

### B. Preparation of Boc-D-Glu(D-Trp-O-Bzl)-OCH₂CH₂CF₃

The Boc-D-Glu(OH)—OCH₂CH₂CF₃ from section A was dissolved in DMF (70 mL). N-Hydroxysuccinimide (2.63 g, 22.8 mmol), EDCl.HCl (4.38 g, 22.8 mmol), H-D-Trp-OBzl.HCl (7.5 g, 22.8 mmol) and DIPEA (4.0 mL, 22.8 mmol) were successively added. The resulting solution was stirred at RT for overnight. The reaction mixture was quenched with de-ionized water and then extracted with EtOAc. The organic layer was washed with brine and dried over anhydrous Na₂SO₄, filtered and concentrated to dryness. The residue was purified by flash column chromatography using a mixture of EtOAc and hexanes (111, v/v) as eluent, thereby affording Boc-D-Glu(D-Trp-O-Bzl)-OCH₂CH₂CF₃ (3.74 g, Y=29%); MS-ESI (m/z): 620 [M+1]⁺.

### C. Preparation of Boc-D-Glu(D-Trp-OH)—OCH₂CH₂CF₃

A mixture of Boc-D-Glu(D-Trp-O-Bzl)-OCH₂CH₂CF₃ from Section B above (3.7 g, 6.0 mmol) and 1.5 g of wet 10% Pd—C in EtOH (150 mL) was hydrogenated under 45 psi of hydrogen gas pressure in a Parr apparatus for 2.5 h. The reaction mixture was filtered, and the filtrate was concentrated to dryness to give the crude Boc-D-Glu(D-Trp-OH)—OCH₂CH₂CF₃.

### D. Preparation of H-D-Glu(D-Trp-OH)—OCH₂CH₂CF₃, Apo878

The Boc-D-Glu(D-Trp-OH)—OCH₂CH₂CF₃ from section C above was dissolved in Et₂O (15 mL), and then a 2M HCl in Et₂O solution (25 mL) was added. The mixture was stirred at RT for overnight. The reaction mixture was concentrated to dryness. The residue was dissolved in de-ionized water (10 mL), and then adjusted to pH 6 using a conc. (28-30%) NH₄OH solution at ice-water bath temperature. The precipitated solid was collected by suction filtration and dried under vacuum to afford H-D-Glu(D-Trp-OH)—OCH₂CH₂CF₃, (Apo878, 1.49 g) as a light purple solid. Yield=58%; ¹H NMR (DMSO-D₆+D₂O, 400 MHz) δ (ppm): 7.51 (d, J=5.1 Hz, 1H), 7.31 (dd, J=8.1, 3.0 Hz, 1H), 7.10 (br. s, 1H), 7.04 (t, J=5.6 Hz, 1H), 6.89-7.01 (m, 1H), 4.18-4.44 (m, 3H), 3.41-3.55 (m, 1H), 3.13-3.29 (m, 1H), 2.89-3.04 (m, 1H), 2.56-2.76 (m, 2H), 2.07-2.18 (m, 2H), 1.78-1.94 (m, 1H), 1.64-1.79 (m, 1H); MS-ESI (m/z): 430 [M+1]⁺.

### Example 2

### General procedure for the preparation of Boc-D-Glu(OH)—O-alkyl

### A. Preparation of Boc-D-Glu(OH)—O-isoamyl

To a suspension of Boc-D-Glu(O-Bzl)-OH (5.48 g, 16.2 mmol), potassium carbonate (4.48 g, 32.5 mmol) and DMF (30 mL) at room temperature was added 1-iodo-3-methylbutane (6.43 g, 32.5 mmol). After the reaction mixture was stirred at room temperature for overnight, the solid was filtered off and washed with ethyl acetate. The filtrate was concentrated by rotary evaporation and the residue was mixed with water. The resulting solid was taken up in hexanes, and the organic solution was washed with water (2×), dried over magnesium sulphate, then filtered. The filtrate was concentrated by rotary evaporation to give Boc-D-Glu(O-Bzl)-O-isoamyl as a white solid (6.64 g) in quantitative yield. ¹H NMR (CDCl₃, 90 MHz) δ ppm: 7.03-7.56 (m, 5H), 5.12 (s, 3H), 3.87-4.50 (m, 3H), 2.25-2.63 (m, 2H), 1.83-2.20 (m, 2H), 1.23-1.75 (m, 12H), 0.91 (d, J=5.85 Hz, 6H).

Boc-D-Glu(O-Bzl)-O-isoamyl (6.20 g, 15.2 mmol) from above and 10% Pd/C (wet, 0.62 g) were mixed in ethyl acetate (80 mL). The reaction mixture was hydrogenated under a hydrogen gas atmosphere using a Parr apparatus at 40 psi hydrogen pressure for 4.5 h. The mixture was filtered through Celite™ and the cake was thoroughly washed with ethyl acetate. The filtrate was concentrated by rotary evaporation to give the title compound Boc-D-Glu(OH)—O-isoamyl as a sticky clear oil in quantitative yield (5.50 g). ¹H NMR (CDCl₃, 400 MHz) δ ppm: 5.18 (d, J=7.1 Hz, 1H), 4.35 (br. s, 1H), 4.18 (t, J=7.1 Hz, 2H), 2.38-2.54 (m, 2H), 2.12-2.27 (m, 1H), 1.84-2.04 (m, 1H), 1.63-1.81 (m, 1H), 1.50-1.63 (m, 2H), 1.45 (s, 9H), 0.93 (d, J=6.1 Hz, 6H).

B. In a similar manner, by replacing 1-iodo-3-methylbutane with other alkyl iodides (methyl iodide, ethyl iodide, 2-iodopropane), the following compounds are prepared:

Boc-D-Glu(OH)—O-Me

Boc-D-Glu (OH)—O-Et

Boc-D-Glu(OH)—O-i-Pr

### Example 3

### Preparation of H-D-Glu(D-Trp-OH)—O-isoamyl (Apo844)

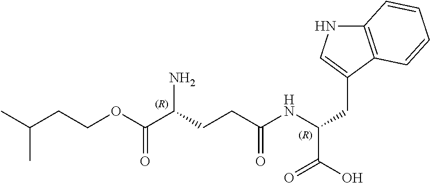

### A. Preparation of Boc-D-Glu(D-Trp-O-Bzl)-O-isoamyl

To a solution of Boc-D-Glu(OH)—O-isoamyl (1.94 g, 6.1 mmol) in N,N-dimethylformamide (15 mL) were added EDCl. HCl (1.17 g, 6.1 mmol), HOBt hydrate (0.93 g, 6.1 mmol) and H-D-Trp-OBzl.HCl (2.02 g, 6.1 mmol), followed by DIPEA (1.18 g, 9.1 mmoL). The reaction mixture was stirred at RT for overnight. The reaction mixture was quenched with a 0.5N HCl solution then extracted with ethyl acetate. The organic layer was successively washed with water, a 0.5M sodium carbonate solution, water and brine, dried over magnesium sulphate and filtered. The filtrate was concentrated in vacuo and the crude product was purified by column chromatography using a solvent gradient consisting of a mixture of ethyl acetate/dichloromethane/hexanes (2/1/7 to 3/1/6, v/v/v) as eluant. Thus Boo-D-Glu(D-Trp-O-Bzl)-O-isoamyl was obtained (2.24 g) as a pale yellow solid. Yield=62%; MS-ESI (m/z): 594 [M+1]⁺.

### B. Preparation of Boc-D-Glu(D-Trp-OH)—O-isoamyl

Boc-D-Glu(D-Trp-O-Bzl)-O-isoamyl from Section A above (2.09 g, 3.5 mmol) and 10% Pd/C (wet, 0.28 g) was mixed in ethyl acetate (50 mL). The reaction mixture was hydrogenated in a Parr apparatus at 10 psi (instrument meter reading) of hydrogen gas pressure for 2.5 h. The mixture was filtered through Celite™ and the cake was washed with ethyl acetate. The filtrate was concentrated by rotary evaporation under reduced pressure. The residue was triturated with hexanes to give Boc-D-Glu(D-Trp-OH)—O-isoamyl (1.49 g) as a pale-pink solid. Yield=84%; MS (m/z): 504 [M+1]⁺.

### C. H-D-Glu(D-Trp-OH)—O-isoamyl (Apo844)

Boc-D-Glu(D-Trp-OH)—O-isoamyl obtained in Section B above (987 mg, 2.0 mmol) was mixed with a 2M HCl in ether solution (30 mL) at RT and stirred for 22.5 h. The reaction mixture was diluted with dichloromethane and concentrated under vacuum by rotary evaporation. The residue was dissolved in water (20 mL) and decolorized with charcoal (1 g), then filtered through Celite™ The filtrate was neutralized with a 1M sodium hydroxide solution to pH 6. The precipitate was filtered, washed with water to give H-D-Glu(D-Trp-OH)—O-isoamyl (Apo844, 652 mg) as off-white solid, Yield=82%; ¹H NMR (DMSO-D₆+D₂O, 400 MHz) δ ppm: 7.50 (d, J=8.1 Hz, 1H), 7.30 (d, J=8.1 Hz, 1H), 7.10 (s, 1H), 7.03 (t, J=7.1 Hz, 1H), 6.86-6.99 (m. 1H), 4.27-4.39 (m, 1H), 4.05 (t, J=6.1 Hz, 2H), 3.23-3.31 (m, 1H), 3.17 (dd, J=14.2, 5.1 Hz, 1H), 2.96 (dd, J=14.2, 8.1 Hz, 1H), 2.14 (t, J=7.1 Hz, 2H), 1.70-1.85 (m, 1H), 1.51-1.68 (m, 2H), 1.38-1.50 (m, 2H), 0.86 (d, J=6.1 Hz, 6H); MS-ESI (m/z): 404 [M+1]⁺.

### Example 4

### Preparation of H-D-Glu(D-Trp-OH)—O-Et hydrochloride salt (Apo836.HCl)

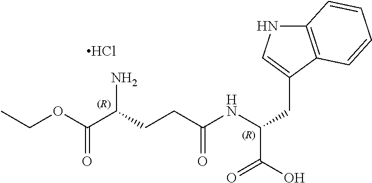

### A. Preparation of Boc-D-Glu(D-Trp-O-Bzl)-O-Et

Proceeding in a similar manner as described under Example 3A, Boc-D-Glu(D-Trp-O-Bzl)-O-Et was prepared in 87% yield. ¹H NMR (DMSO-D₆, 400 MHz) δ ppm: 10.87, (s, 1H), 8.35 (d, J=7.2 Hz, 1H), 7.48 (d, J=7.8 Hz, 1H), 7.35 (d, J=7.9 Hz, 1H). 7.29-7.33 (m, 3H), 7.23 (d, J=7.7 Hz, 1H), 7.09-7.22 (m, 3H), 7.08 (t, J=7.6 Hz, 1H), 6.98 (t, J=7.7 Hz, 1H), 4.98-5.06 (m, 2H), 4.55 (apparent q, J=7.3 Hz, 1H), 4.04-4.11 (m, 2H), 3.90-3.95 (m, 1H), 3.04-3.19 (m, 2H), 2.18-2.23 (m, 2H), 1.84-1.89 (m, 1H), 1.70-1.77 (m, 1H). 1.38 (s, 9H), 1.16 (t, J=7.1 Hz, 3H); MS-ESI (m/z): 552 [M+1]⁺.

### B. Preparation of Boc-D-Glu(D-Trp-OH)—O-Et

Proceeding in a similar manner as described under Example 3B, Boc-D-Glu(D-Trp-OH)—O-Et was prepared in quantitative yield. ¹H NMR (DMSO-D₆, 400 MHz) δ ppm: 12.62 (br. 1H), 10.82, (s, 1H), 8.10 (d, J=7.7 Hz, 1H), 7.52 (d, J=7.8 Hz, 1H), 7.33 (d, J=8.0 Hz, 1H), 7.23 (d, J=7.5 Hz, 1H), 7.12 (s, 1H), 7.06 (t, J=7.3 Hz, 1H), 6.98 (t, J=7.5 Hz, 1H), 4.45 (apparent q, J=7.7 Hz, 1H), 4.03-4.11 (m, 2H), 3.87-3.92 (m, 1H), 3.13-3.18 (m, 1H), 2.96-3.03 (m, 1H), 2.13-2.20 (m, 2H), 1.82-1.88 (m, 1H), 1.69-1.75 (m, 1H), 1.38 (s, 9H), 1.17 (t, J=7.1 Hz, 3H); MS-ESI (m/z): 462 [M+1]⁺.

### C. Preparation of H-D-Glu(D-Trp-OH)—O-Et.HCl (Apo836.HCl)

To an ice-cooled solution of Boc-D-Glu(D-Trp-OH)—O-Et (4.55 g, 9.8 mmol) obtained in Section B above in dichloromethane (100 mL) was bubbled HCl gas for 15 min. The reaction mixture was concentrated under vacuum by rotary evaporation to give H-D-Glu(D-Trp-OH)—O-Et hydrochloride (Apo836.HCl, 4.0 g) as a foamy solid. ¹H NMR (DMSO-D₆, 400 MHz) δ ppm: 12.68 (br. s, 1H), 10.90, (s, 1H), 8.66 (br, s, 3H), 8.33 (d, J=7.8 Hz, 1H), 7.52 (d, J=7.8 Hz, 1H), 7.33 (d, J=8.0 Hz, 1H), 7.12 (d, J=1.5 Hz, 1H), 7.06 (t, J=7.2 Hz, 1H), 6.98 (t, J=7.2 Hz, 1H), 4.47 (apparent q, J=4.8 Hz, 1H), 4.13-4.19 (m, 2H), 3.90 (br, 1H), 3.16-3.20 (m, 1H), 2.98-3.04 (m, 1H), 2.29-2.33 (m, 2H), 1.94-1.98 (m, 2H), 1.20 (t, J=7.1 Hz, 3H); MS-ESI (m/z): 362 [M+1]⁺ (free base).

### Example 5

### Preparation of H-D-Glu(D-Trp-OH)—O-i-Pr, Apo846

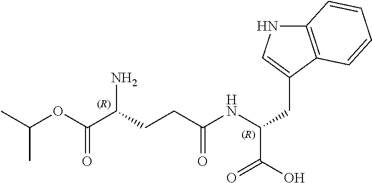

### A. Preparation of Boc-D-Glu(D-Trp-O-Bzl)-O-i-Pr

The Boc-D-Glu(OH)—O-f-Pr was dissolved in DMF (60 mL), and then N-hydroxysuccinimide (2.87 g, 24.9 mmol), EDCl.HCl (4.77 g, 24.9 mmol) and DIPEA (4.3 mL, 24.9 mmol) were successively added. After stirring at RT for 3.5 h, H-D-Trp-OBzl.HCl (7.55 g, 22.8 mmol) was added followed by DIPEA (4.3 mL, 24.9 mmol). The mixture was stirred for overnight. The reaction mixture was quenched with de-ionized water and then extracted with EtOAc. The EtOAc layer was washed with brine, dried over anhydrous Na₂SO₄, filtered and concentrated to dryness to give crude Boc-D-Glu(D-Trp-O-Bzl)-O-i-Pr.

### B. Preparation of Boc-D-Glu(D-Trp-OH)—O-i-Pr

The crude product from section A above was dissolved in isopropanol (100 mL), and 0.7 g of wet 10% Pd—C was added. The mixture was hydrogenated under 40 psi hydrogen gas pressure in a Parr apparatus for 2 h. The mixture was filtered, and the filtrate was evaporated to dryness to give crude Boc-D-Glu(D-Trp-OH)—O-i-Pr.

### C. Preparation of H-D-Glu(D-Trp-OH)—O-i-Pr, Apo846

The residue from section B above was dissolved in Et₂O (20 mL), and a 2M HCl in Et₂O solution (15 mL) was added. The mixture was stirred at RT for overnight. The reaction mixture was concentrated to dryness. The residue was dissolved in de-ionized water (6 mL), and the pH of the mixture was adjusted to about 5.5 using a 6N NaOH solution at ice-water bath temperature to afford a crude product. The crude material was purified using the Biotage instrument with reverse C18 column to afford the H-D-Glu(D-Trp-OH)—O-i-Pr (Apo846, 1.06 g) as white solid. Yield=14%; ¹H NMR (DMSO-D₆+D₂O, 400 MHz) δ ppm: 7.52 (d, J=7.1 Hz, 1H), 7.31 (d, J=8.1 Hz, 1H), 7.10 (s, 1H), 7.04 (t, J=7.6 Hz, 1H), 6.93-6.99 (m, 1H), 4.90 (quin, J=6.1 Hz, 1H), 4.35 (dd, J=8.6, 4.5 Hz, 1H), 3.35 (dd, J=8.1, 5.1 Hz, 1H), 3.18 (dd, J=14.7, 4.5 Hz, 1H), 2.96 (dd, J=14.7, 8.6 Hz, 1H), 2.16 (t, J=7.6 Hz, 2H), 1.73-1.85 (m, 1H), 1.59-1.72 (m, 1H), 1.18 (m, 6H); MS-ESI (m/z): 376 [M+1]⁺.

### Example 6

### Preparation of H-D-Glu(D-Trp-OH)—O-Bzl, Apo829

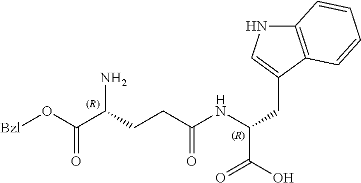

### A. Preparation of Boc-D-Glu(D-Trp-OH)—O—Bzl

Boc-D-Glu-OBzl (11.24 g, 33.3 mmol) was mixed with HOSu (3.83 g, 33.3 mmol) and EDCl hydrochloride (6.38 g, 33.3 mmol) in DMF (80 mL) at room temperature and stirred for overnight. D-Trp-OH (10.2 g, 50 mmol) was added all at once and the reaction mixture was stirred at room temperature for another 6 h. The mixture was then quenched with a 0.5N HCl solution (250 mL) as a sticky solid formed. The liquid fraction was decanted and the residual sticky solid was dissolved in ethyl acetate (200 mL). The ethyl acetate layer was washed with a 0.5 N HO solution (100 mL×2), water (100 mL×2) and brine, dried over MgSO₄ and filtered. The filtrate was concentrated in vacuo by rotary evaporation and the residue was triturated with ether to give Boc-D-Glu(D-Trp-OH)—O—Bzl as a white solid (6.60 g). The mother liquid was concentrated and triturated with 10% ethyl acetate in hexanes to give a second crop of product as off-white solid (7.23 g). Combined yield=13.82 g (79%); ¹H NMR (DMSO-D₅, 400 MHz) δ (ppm): 10.82 (br. s, 1H), 8.09 (d, J=7.1 Hz, 1H), 7.51 (d, J=7.1 Hz, 1H), 7.27-7.41 (m, 7H), 7.11 (s, 1H), 7.05 (t, J=7.1 Hz; 1H), 6.92-7.00 (m, 1H), 5.01-5.21 (m, 2H), 4.39-4.51 (m, 1H), 3.94-4.06 (m, 1H), 3.08-3.19 (m, 1H), 2.93-3.06 (m, 1H), 2.05-2.26 (m, 2H), 1.82-1.98 (m, 1H), 1.67-1.79 (m, 1H), 1.37 (s, 9H); MS (m/z) 524 [M+1]⁺.

### B. Preparation of H-D-Glu(D-Trp-OH)—O—Bzl hydrochloride salt, Apo829.HCl

Boc-D-Glu(D-Trp-OH)—O—Bzl (6.60 g, 12.6 mmol) was mixed with 4M HCl in dioxane (30 mL) and ethyl acetate (30 mL) at room temperature. After stirring for 50 min, an additional 4M HCl in dioxane (10 mL) was added. The reaction mixture was stirred for another 140 min, then concentrated in vacuo by rotary evaporation. The residue was triturated with ethyl acetate and the mixture was stirred for overnight. The resulting sticky solid was then triturated with a 10% ethyl acetate in hexanes mixture to give H-D-Glu(D-Trp-OH)—O—Bzl hydrochloride salt as an off-white solid (5.15 g). Yield=88%; ¹H NMR (400 MHz, DMSO-C₆) δ ppm: 10.88 (br. s, 1H), 8.53 (br. s, 3H), 8.31 (d, J=8.1 Hz, 1H), 7.51 (d, J=8.1 Hz, 1H), 7.29-7.43 (m, 6H), 7.14 (s, 1H), 7.05 (t, J=7.6 Hz, 1H), 6.97 (m, 1H), 5.24 (d, J=12.4 Hz, 1H), 5.16 (d, J=12.4 Hz, 1H), 4.41-4.52 (m, 1H), 3.96-4.13 (m, 2H)_{;} 3.16 (dd, J=14.2, 5.1 Hz, 1H), 3.01 (dd, J=14.7, 8.6 Hz, 1H), 2.26-2.36 (m, 2H), 1.92-2.03 (m, 2H). In a duplicate experiment, the desired HCl product (5.95 g) was obtained in 94% from the deprotection reaction of Boc-D-Glu(D-Trp-OH)—O—Bzl with 4M HCl in dioxane (40 mL).

### C. Preparation of H-D-Glu(D-Trp-OH)—O—Bzl, Apo829

The combined products obtained in Step B above (11.0 g) was dissolved in water (75 mL) and filtered. The ice-water cooled filtrate was then neutralized with a 6N NaOH solution to pH about 6. The resulting precipitate was filtered, washed with water to afford H-D-Glu(D-Trp-OH)—O—Bzl (Apo829). The wet product was triturated with ether (100 mL) for an hour, and was then collected via suction filtration. Analysis by HPLC indicated an AUC purity of 96.7%. Further purification was carried out. Thus, the product was mixed with ethyl acetate (20 mL) and 4M HCl in dioxane (20 mL) to form a clear solution. The solution was concentrated and the residue was triturated with ethyl acetate and hexanes. The solid was dissolved in water (300 mL) and cooled in an ice-water bath, then neutralized with a 6N NaOH solution to pH about 6. The precipitated fine solid was collected by suction filtration, washed with water and ether to give H-D-Glu(D-Trp-OH)—O—Bzl, Apo829 (6.05 g). Yield=60%; HPLC purity (AUC)=98.8%; ¹H NMR (400 MHz, DMSO-D₆) δ ppm: 10.81 (br. s, 1H), 8.05 (d, J=8.1 Hz, 1H), 7.51 (d, J=8.1 Hz, 1H), 7.27-7.41 (m, 6H), 7.12 (s, 1H), 7.04 (t, J=7.6 Hz, 1H), 6.91-6.99 (m, 1H), 5.10 (s, 2H), 4.35-4.46 (m, 1H), 3.30-3.36 (m, 1H), 3.16 (dd, J=14.7, 4.6 Hz, 1H), 2.97 (dd, J=14.7, 8.6 Hz, 1H), 2.12-2.24 (m, 2H), 1.74-1.87 (m, 1H), 1.59 (dd, J=14.2, 7.1 Hz, 1H); MS-ESI (m/z) 424 [M+1]⁺.

### D. Proceeding in a similar manner as above, H-D-Glu(D-Trp-OH)—OCH₂CF₃, Apo865 was prepared

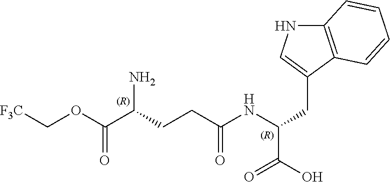

Yield=8.3 g (49%); ¹H NMR (DMSO-D₆+D₂O, 400 MHz) δ (ppm): 7.50 (d, J=6.8 Hz, 1H), 7.32 (d, J=7.0 Hz, 1H), 7.10 (s, 1H), 7.03-7.09 (m, 1H), 6.94-7.03 (m, 1H), 4.70-4.75 (m, 2H), 4.38-4.41 (m, 1H), 3.49-3.51 (m, 1H), 3.14-3.19 (m, 1H), 2.93-2.99 (m, 1H), 2.17-2.24 (m, 2H), 1.80-1.86 (m, 1H), 1.60-1.68 (m, 1H); MS-ESI (m/z): 416 [M+1]⁺.

### Example 7

### Preparation of H-D-Glu(D-Trp-O—CH(CH₃)—O—CO—O-cyclohexyl)-OH (Apo843)

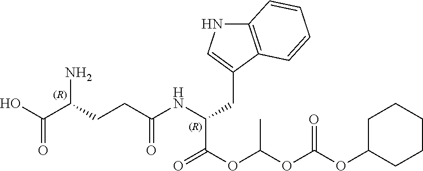

### A. Preparation of Cbz-D-Glu(D-Trp-OH)—O—Bzl

Cbz-D-Glu(OH)—O—Bzl (18.57 g, 50 mmol), HOSu (5.76 g, 50 mmol) and EDCl.HCl (9.59 g, 50 mmol) were mixed in DMF (100 mL) at ice-water bath temperature. The reaction mixture was allowed to warm to RT then stirred at RT for overnight. The reaction mixture was cooled again in an ice-water bath and D-Trp-OH (10.21 g, 50 mmol) was added. The mixture was then stirred at RT for 6 h. The mixture was poured into a beaker containing a mixture of 0.5N HCl (200 mL) and ice chunks. The mixture was extracted with ethyl acetate twice (200 mL+100 mL). The organic layers were combined and washed with water (100 mL×3) and brine (100 mL), dried over magnesium sulphate and filtered. The filtrate was concentrated by rotary evaporation under reduced pressure and the resulting solid was triturated with a mixture of ether and hexanes. Cbz-D-Glu(D-Trp-OH)—O—Bzl (24.5 g) was obtained as a white solid after suction filtration. Yield=88%; ¹H NMR (DMSO-D₅, 400 MHz) δ ppm: 12.55 (br. s, 1H), 10.81 (s, 1H), 8.11 (d, J=8.1 Hz, 1H), 7.78 (d, J=8.1 Hz, 1H), 7.51 (d, J=7.1 Hz, 1H), 7.19-7.45 (m, 11H), 7.11 (s, 1H), 7.05 (t, J=7.6 Hz, 1H), 6.96 (t, J=7.6 Hz, 1H), 4.92-5.22 (m, 4H), 4.37-4.55 (m, 1H), 3.99-4.17 (m, 1H), 3.14 (dd, J=14.7, 5.6 Hz, 1H), 2.92-3.07 (m, 1H), 2.08-2.33 (m, 2H), 1.85-2.07 (m, 1H), 1.64-1.85 (m, 1H); MS-ESI (m/z): 558 [M+1]⁺.

### B. Preparation of Cbz-D-Glu(D-Trp-O—CH(CH₃)—O—CO—O-cyclohexyl)-O-Bzl

To a mixture of Cbz-D-Glu(D-Trp-OH)—O—Bzl (6.00 g, 10.8 mmol), potassium carbonate (5.94 g, 43.0 mmol) and sodium iodide (28.50 g, 190.1 mmol) in N,N-dimethylformamide (40 mL) was added 1-chloroethyl-cyclohexyl carbonate (8.90 g, 43.0 mmol) at RT. After stirring at 30° C. for overnight, the reaction mixture was diluted with ethyl acetate. The mixture was then washed with water (×3) and brine. The residue was subjected to purification by column chromatography on silica gel using a solvent gradient consisting of a mixture of ethyl acetate in hexanes (20 to 40%) as eluant. Fractions rich in product were combined, and volatiles were removed in vacuo. Thus, the alkylated product CBz-D-Glu(D-Trp-O—CH(CH₃)—O—CO—O-cyclohexyl)-O-Bzl (3.77 g) was obtained as a pale-yellow foam. Yield=48%; ¹H NMR (DMSO-D₆, 400 MHz) δ ppm: 10.86 (s, 1H), 8.35 (dd, J=17.7, 7.6 Hz, 1H), 7.78 (t, J=7.6 Hz, 1H), 7.46 (t, J=8.1 Hz, 1H), 7.34 (br. s, 11H), 7.14 (d, J=3.0 Hz, 1H), 7.06 (t, J=7.6 Hz, 1H), 6.94-7.00 (m, 1H), 6.62 (q, J=5.1 Hz, 0.5H), 6.51 (q, J=5.1 Hz, 0.5H), 5.12 (d, J=3.0 Hz, 2H), 4.97-5.09 (m, 2H), 4.40-4.59 (m, 2H), 4.05-4.15 (m, 1H), 2.93-3.18 (m. 2H), 2.15-2.27 (m, 2H), 1.91-1.97 (m, 1H), 1.71-1.86 (m, 3H), 1.57-1.68 (m, 2H), 1.13-1.49 (m, 9H); MS-ESI (m/z): 728 [M+1]⁺.

### C. Preparation of H-D-Glu(D-Trp-O—CO—O-cyclohexyl)-OH (Apo843)

Cbz-D-Glu(D-Trp-O—CH(CH₃)—O—CO—O-cyclohexyl)-O-Bzl obtained in Section B above (3.67 g, 5.0 mmol) and 10% Pd—C(wet, 1.16 g) was mixed in ethanol (100 mL). The mixture was hydrogenated in a Parr apparatus under a blanket of hydrogen at 15-25 psi of hydrogen pressure for 3 h. The mixture was filtered through Ceilte™ and the cake was washed with ethanol. The filtrate was concentrated by rotary evaporation under reduced pressure and the residue was triturated with ether to give the title compound H-D-Glu(D-Trp-O—CH(CH₃)—O—CO—O-cyclohexyl)-OH (Apo843, 2.00 g) as a white solid. Yield=78%; ¹H NMR (DMSO-D₆+D₂O, 400 MHz,) δ ppm: 7.46 (t, J=9.1 Hz, 1H), 7.34 (d, J=8.1 Hz, 1H), 7.17 (s, 1H), 7.07 (t, J=7.6 Hz, 1H), 6.90-7.03 (m, 1H), 6.58-6.68 (m, 0.5H), 6.42-6.57 (m, 0.5H), 4.49-4.61 (m, 1H), 4.32-4.49 (m, 1H), 3.20-3.30 (m, 1H), 2.89-3.20 (m, 2H), 2.09-2.38 (m. 2H), 1.75-1.92 (m, 4H), 1.62 (br. s, 2H), 1.12-1.54 (m, 9H); MS-ESI (m/z): 504 [M+1]⁺.

### Example 8

### Preparation of H-D-Glu(D-Trp-O—CH(CH₃)—O—CO—O-Et)-OH, Apo888

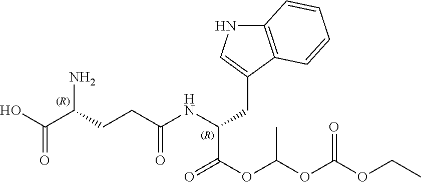

Proceeding in a similar manner as described under example 7B, Cbz-D-Glu(D-Trp-O—CH(CH₃)—O—CO—O-Et)-O-Bzl (1.65 g, yield=61%) was prepared from the reaction of Cbz-D-Glu(D-Trp-OH)—O—Bzl from example 7A (2.24 g, 4.0 mmol) with 1-chloroethyl ethyl carbonate (1.22 g, 8.0 mmol) in the presence of potassium carbonate (1.10 g, 8.0 mmol) and sodium iodide (2.40 g, 16.0 mmol) in N,N-dimethylformamide (20 mL) at 50° C. for overnight. ¹H NMR (CD₃OD, 400 MHz) δ ppm: 10.86 (br. s, 1H), 8.24-8.47 (m, 1H), 7.79 (t, J=7.6 Hz, 1H), 7.40-7.52 (m, 1H), 7.24-7.42 (m, 11H), 7.14 (d, J=5.1 Hz, 1H), 7.06 (t, J=7.6 Hz, 1H), 6.93-7.02 (m, 1H), 6.57-6.67 (m, 0.5H), 6.44-6.56 (m, 0.5H), 5.12 (d, J=3.0 Hz, 2H), 4.97-5.09 (m, 2H), 4.41-4.49 (m, 1H), 4.05-4.18 (m, 3H), 2.95-3.17 (m, 2H), 2.13-2.29 (m, 2H), 1.88-1.98 (m, 1H), 1.76 (dd, J=14.1, 8.1 Hz, 1H), 1.42 (d, J=6.1 Hz, 1.5H), 1.13-1.25 (m, 4.5H); MS-ESI (m/z): 674 [M+1]⁺.

Proceeding in a similar manner as described under example 7C, H-D-Glu(D-Trp-O—CH(CH₃)—O—CO—O-Et)-OH, Apo888, (623 mg, yield=56%) was prepared from the deprotection of Cbz-D-Glu(D-Trp-O—CH(CH₃)—O—CO—O-Et)-O-Bzl (1.65, 2.5 mmol) via hydrogenation with 10% Pd/C (wet, 0.5 g) in ethanol (100 mL) under a blanket of hydrogen. ¹H NMR (DMSO-D₅, 400 MHz) δ ppm: 10.96 (br. s, 1H), 8.76 (br. s, 1H), 7.41-7.52 (m, 1H), 7.34 (d, J=8.1 Hz, 1H), 7.20 (br. s, 1H), 7.02-7.14 (m, 1H), 6.90-7.02 (m, 1H), 6.57-6.69 (m, 0.5H), 6.44-6.57 (m, 0.5H), 4.36-4.49 (m, 1H), 4.14 (q, J=7.1 Hz, 2H), 2.93-3.17 (m, 3H), 2.18-2.37 (m, 2H), 1.71-1.99 (m, 2H), 1.45 (d, J=5.1 Hz, 1.5H), 1.17-1.27 (m, 4.5H). MS m/z: 450 [M+1]⁺.

### Example 9

### Preparation of H-D-Glu(D-Trp-O—CH(CH₃)—O—CO—O-i-Pr)-OH (Apo891)

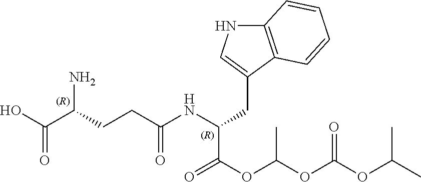

Proceeding in a similar manner as described under example 7B, Cbz-D-Glu(D-Trp-O—CH(CH₃)—O—CO—O-i-Pr)-O-Bzl (1.54 g, yield=56%) was prepared from the reaction of Cbz-D-Glu(D-Trp-OH)—O—Bzl from Example 7A above (2.24 g, 4.0 mmol) with 1-chloroethyl isopropyl carbonate (1.33 g, 8.0 mmol) in presence of potassium carbonate (1.10 g, 8.0 mmol) and sodium iodide (2.40 g, 16.0 mmol) in N,N-dimethylformamide (20 mL) at 50° C. for overnight. ¹H NMR (CD₃OD, 400 MHz) δ ppm: 7.43-7.55 (m, 1H), 7.31 (br, s, 11H), 7.03-7.12 (m, 2H), 6.92-7.04 (m, 1H), 6.67-6.78 (m, 0.5H), 6.53-6.67 (m, 0.5H), 5.00-5.18 (m, 4H), 4.76-4.85 (m, 1H), 4.63-4.76 (m, 1H), 4.12-4.23 (m, 1H), 3.20-3.25 (m, 1H), 3.05-3.19 (m, 1H), 2.18-2.31 (m, 2H), 2.01-2.13 (m, 1H), 1.77-1.93 (m, 1H), 1.45 (d, J=5.1 Hz, 1.5H), 1.18-1.31 (m, 7.5H); MS-ESI (m/z): 688 [M+1]⁺.

Proceeding in a similar manner as described under example 7C, H-D-Glu(D-Trp-O—CH(CH₃)—O—CO—O-i-Pr)-OH, Apo891, (0.36 g, yield=36%) was prepared from the hydrogenation of Cbz-D-Glu(D-Trp-O—CH(CH₃)—O—CO—O-i-Pr)-O-Bzl (1.50, 2.2 mmol) with 10% Pd/C (wet, 0.56 g) in ethanol (50 mL). ¹H NMR (CD₃OD, 400 MHz) δ ppm: 7.47-7.55 (m, 1H), 7.33 (d, J=8.1 Hz, 1H), 7.05-7.15 (m, 2H), 6.97-7.05 (m, 1H), 6.70-6.78 (m, 0.5H), 6.58-6.66 (m, 0.5H), 4.80-4.86 (m, 1H), 4.66-4.74 (m, 1H), 3.58-3.65 (m, 1H), 3.24-3.37 (m. 1H), 3.04-3.21 (m, 1H), 2.32-2.50 (m, 2H), 1.93-2.11 (m, 2H), 1.49 (d, J=5.1 Hz, 1.5H), 1.21-1.32 (m, 7.5H); MS-ESI (m/z): 464 [M+1]⁺.

### Example 10

### Preparation of H-D-Glu(D-Trp-O—CH₂CO—N(CH₃)₂)—OH, Apo893

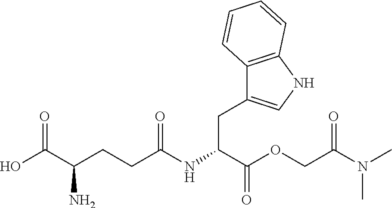 [SMILES: CN(C)C(=O)COC(=O)[C@@H](Cc1c[nH]c2ccccc12)NC(=O)CC[C@@H](N)C(=O)O]

Proceeding in a similar manner as described under example 7B, Cbz-D-Glu(D-Trp-O—CH₂CO—N(CH₃)₂)—O-Bzl (1.11 g, yield=43%) was prepared from the reaction of Cbz-D-Glu(D-Trp-OH)—O—Bzl from Example 7A above (2.24 g, 4.0 mmol) with 2-chloro-N,N-dimethylacetamide (0.73 g, 6.0 mmol) in the presence of potassium carbonate (1.10 g, 8.0 mmol) in N,N-dimethylformamide (20 mL). ¹H NMR (DMSO-D₆, 400 MHz) δ ppm: 10.84 (br. s, 1H), 8.31 (d, J=7.1 Hz, 1H), 7.78 (d, J=8.1 Hz, 1H), 7.49 (d, J=8.1 Hz. 1H), 7.22-7.44 (m, 11H), 7.17 (s, 1H), 7.02-7.15 (m, 1H), 6.98 (d, J=7.1 Hz, 1H), 4.94-5.18 (m, 4H), 4.85 (d, J=14.8 Hz, 1H), 4.75 (d, J=14.8 Hz, 11-1), 4.48-4.60 (m, 1H), 3.99-4.14 (m, 1H), 3.28-3.32 (m, 1H), 2.95-3.07 (m, 1H), 2.89 (s, 3H), 2.82 (s, 3H), 2.07-2.27 (m, 2H), 1.82-1.98 (m, 1H), 1.64-1.80 (m, 1H); MS-ESI (m/z): 643 [M+1]⁺.

Proceeding in a similar manner as described under Example 7C, H-D-Glu(D-Trp-O—CH₂CO—N(CH₃)₂)—OH, Apo893, (0.54 g, yield=75%) was prepared from the deprotection of Cbz-D-Glu(D-Trp-O—CH₂CO—N(CH₃)₂)—O-Bzl (1.10 g, 1.7 mmol) via hydrogenation with 10% Pd/C (wet, 0.62 g) in ethanol (100 mL) under a blanket of hydrogen. ¹H NMR (DMSO-D₆, 400 MHz) δ ppm: 10.98 (br. s, 1H), 8.82 (d, J=7.1 Hz, 1H), 7.51 (d, J=8.1 Hz, 1H), 7.34 (d, J=8.1 Hz, 1H), 7.22 (s, 1H), 7.02-7.13 (m, 1H), 6.94-7.02 (m, 1H), 4.89 (d, J=14.8 Hz, 1H), 4.78 (d, J=14.8 Hz, 1H), 4.46-4.58 (m, 1H), 3.26-3.37 (m, 2H), 2.95-3.08 (m, 1H), 2.90 (s, 3H), 2.83 (s, 3H), 2.24-2.36 (m, 1H), 2.11-2.24 (m, 1H), 1.72-1.89 (m, 2H); MS-ESI (m/z): 419 [M+1]⁺.

### Example 11

### Preparation of H-D-Glu(D-Trp-O-mofetil)-OH.HCl salt (Apo849.HCl)

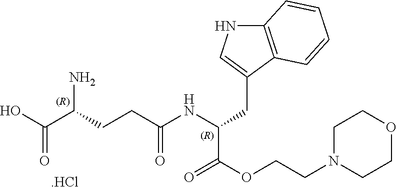

Proceeding in a similar manner as described under example 7B, Cbz-D-Glu(D-Trp-O-mofetil)-O-Bzl hydrochloride (4.53 g, yield=64%) was prepared from the reaction of Cbz-D-Glu(D-Trp-OH)—O—Bzl (2.24 g, 4.0 mmol) with 2-morpholinoethyl methanesulfonate, which was prepared from 2-morpholinoethanol (1.97 g, 15.0 mmol) and methanesulfonyl chloride (1.72 g, 15.0 mmol), in the presence of potassium carbonate (1.10 g, 8.0 mmol) in N,N-dimethylformamide (15 mL). ¹H NMR (DMSO-D₆, 400 MHz) d ppm: 10.91 (br. s, 2H), 8.49 (d, J=7.1 Hz, 1H), 7.81 (d, J=8.1 Hz, 1H), 7.49 (d, J=8.1 Hz, 1H), 7.35 (d, J=3.0 Hz, 11H), 7.17 (s, 1H), 7.07 (t, J=7.6 Hz, 1H), 6.93-7.03 (m, 1H), 5.12 (br. s, 2H), 4.98-5.10 (m, 2H), 4.53 (q, J=7.1 Hz, 1H), 4.22-4.41 (m, 2H), 4.06-4.15 (m, 1H), 3.61-3.89 (m, 4H), 3.01-3.34 (m, 6H), 2.86-3.01 (m, 2H), 2.21-2.30 (m, 2H), 1.89-2.03 (m, 1H), 1.69-1.83 (m, 1H); MS-ESI (m/z): 671 [M+1]⁺.

Proceeding in a similar manner as described under example 7C, H-D-Glu(D-Trp-O-mofetil)-OH hydrochloride salt. Apo849.HCl, (1.01 g, yield=74%) was prepared from the hydrogenation of Cbz-D-Glu(D-Trp-O-mofetil)-O-Bzl hydrochloride (2.00 g, 2.8 mmol) with 10% Pd—C(wet, 1.00 g) in methanol (100 mL). ¹H NMR (DMSO-D₆, 400 MHz) δ ppm: 11.20 (br. s, 1H), 8.87 (d, J=7.1 Hz, 1H), 7.74 (d, J=8.1 Hz, 1H), 7.59 (d, J=8.1 Hz, 1H), 7.44 (s, 1H), 7.32 (t, J 7.1 Hz, 1H), 7.24 (t, J=7.6 Hz, 1H), 4.75 (q, J=7.1 Hz, 1H), 4.35-4.50 (m, 2H), 3.96-4.06 (m, 1H), 3.88 (br. s, 4H), 3.41 (dd, J=14.1, 6.1 Hz, 1H), 3.31 (dd, J=14.1, 8.1 Hz, 1H), 2.91-3.08 (m, 2H), 2.85 (br. s, 4H), 2.45-2.66 (m, 2H), 2.12-2.28 (m, 2H); MS-ESI (m/z): 447 [M+1]⁺ (free base).

### Example 12

### Preparation of H-D-Glu(D-Trp-OCH₂O—CO-Ph)-OH, Apo883

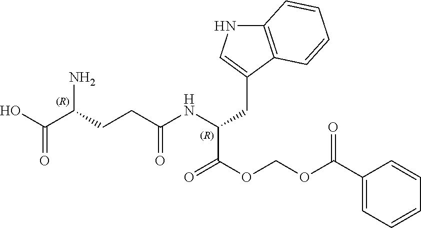

Proceeding in a similar manner as described under example 7B above, Cbz-D-Glu(D-Trp-OCH₂O—CO-Ph)-O-Bzl (2.45 g) was obtained after work-up from the reaction of a mixture of chloromethyl benzoate (1.04 g, 6.1 mmol), sodium iodide (4.6 g, 30.7 mmol) and Cbz-D-Glu(D-Trp-OH)—O—Bzl (2.29 g, 4.1 mmol) in presence of DIPEA (0.82 mL, 4.7 mmol) in acetone (80 mL). Yield 86%; ¹H NMR (DMSO-D₆, 400 MHz) δ ppm: 10.86 (br. s, 1H), 8.40 (d, J=7.1 Hz, 1H), 7.94 (d, J=7.1 Hz, 2H), 7.78 (d, J=8.1 Hz, 1H), 7.70 (t, J=7.6 Hz, 1H), 7.51-7.61 (m, 2H), 7.47 (d, J=8.1 Hz, 1H), 7.24-7.42 (m, 11H), 7.14 (d, J=2.0 Hz, 1H), 7.05 (1, J=7.6 Hz, 1H), 6.89-7.00 (m, 1H), 5.90-6.00 (m, 2H), 4.97-5.18 (m, 4H), 4.47-4.59 (m, 1H), 4.05-4.11 (m, 1H), 3.11-3.21 (m, 1H), 3.00-3.11 (m, 1H), 2.12-2.31 (m, 2H), 1.87-2.02 (m, 1H), 1.67-1.83 (m, 1H); MS-ESI (m/z): 692 [M+1]⁺.

Proceeding in a similar manner as described under example 7C, the hydrogenolysis of Cbz-D-Glu(D-Trp-OCH₂O—CO-Ph)-O-Bzl (2.2 g, 3.2 mmol) obtained above with 2.2 g of wet 10% Pd—C in MeOH (150 mL) under 45 psi hydrogen pressure in a Parr apparatus for 5.5 h afforded crude H-D-Glu(D-Trp-OCH₂O—CO-Ph)-OH. Purification of the crude product (1.2 g) by flash column chromatography on silica gel using a mixture of i-PrOH and H₂O (85/15, v/v) as eluent afforded the title compound H-D-Glu(D-Trp-OCH₂O—CO-Ph)-OH (Apo883, 410 mg). Yield=28%; ¹H NMR (DMSO-D₆+D₂O, 400 MHz): δ ppm: 7.91 (d, J=8.1 Hz, 2H), 7.65-7.74 (m, 1H), 7.54 (t, J=7.6 Hz, 2H), 7.44 (d, J=8.1 Hz, 1H), 7.31 (d, J=8.1 Hz, 1H), 7.13 (s, 1H), 7.04 (t, J=7.6 Hz, 1H), 6.90-6.98 (m, 1H), 5.86-5.97 (m, 2H), 4.45-4.55 (m, 1H), 3.30 (t, J=6.1 Hz, 1H), 2.97-3.19 (m, 2H), 2.24 (t, J=7.6 Hz, 2H), 1.75-1.93 (m, 2H); MS-ESI (m/z): 468 [M+1]⁺.

### Example 13

### Preparation of H-D-Glu(D-Trp-OCH₂O—CO-[pent-3-yl])-OH, Apo889

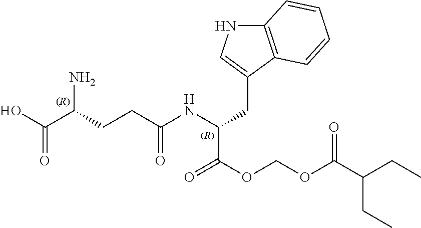

In a similar manner as described under Example 12, by replacing chloromethyl benzoate with chloromethyl 2-ethylbutanoate, H-D-Glu(D-Trp-OCH₂O—CO-[pent-3-yl])-OH (Apo889) was prepared. ¹H NMR (DMSO-D₆+D₂O, 400 MHz) δ ppm: 7.46 (d, J=7.1 Hz, 1H), 7.35 (d, J=8.1 Hz, 1H), 7.18 (s, 1H), 7.04-7.12 (m, 1H), 6.96-7.03 (m, 1H), 5.74-5.82 (m, 1H), 5.67-5.74 (m, 1H), 4.45 (dd, J=9.1, 5.1 Hz. 1H), 3.27 (t, J=6.6 Hz, 1H), 3.06-3.17 (m, 1H), 2.99 (dd, J=14.7, 9.6 Hz, 1H), 2.14-2.32 (m, 3H), 1.72-1.91 (m, 2H), 1.38-1.58 (m, 4H), 0.74-0.87 (m, 6H); MS-ESI (m/z): 462[M+1]⁺.

### Example 14

### Preparation of H-D-Glu(D-Trp-OCH₂O—CO—C(CH₃)₂—CH₂CH₂CH₃)—OH, Apo895

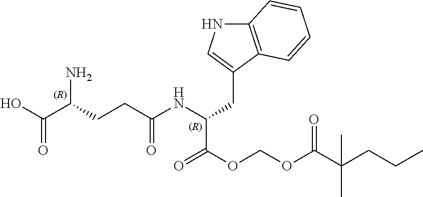

Proceeding in a similar manner as described in Example 12, by replacing chloromethyl benzoate with chloromethyl 2,2-dimethylpentanoate, H-D-Glu(D-Trp-OCH₂O—CO—C(CH₃)₂—CH₂CH₂CH₃)—OH (Apo895) was prepared. ¹H NMR (DMSO-D₆+D₂O, 400 MHz) δ ppm: 7.44 (d, J=8.1 Hz, 1H), 7.34 (d, J=8.1 Hz, 1H), 7.17 (s, 1H), 7.04-7.14 (m, 1H), 6.93-7.04 (m, 1H), 5.61-5.83 (m, 2H), 4.36-4.53 (m, 1H), 3.20-3.38 (m, 1H), 3.06-3.21 (m, 1H), 2.91-3.06 (m, 1H), 214-2.36 (m, 2H), 1.72-1.94 (m, 2H), 1.25-1.52 (m, 2H), 0.91-1.26 (m, 8H), 0.68-0.88 (m, 3H); MS-ESI (m/z): 476 [M+1]⁺.

### Example 15

### Preparation of H-D-Glu(D-Trp-OCH₂CH₂CF₃)—OH, Apo877

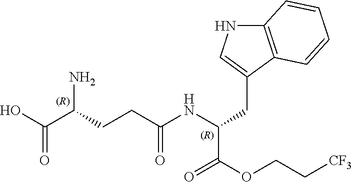

In a similar manner as described under example 12, by replacing chloromethyl benzoate with CF₃CH₂CH₂I, Cbz-D-Glu(D-Trp-OCH₂CH₂CF₃)—O-Bzl (2.0 g, yield=86%) was obtained after purification by flash column chromatography on silica gel. Hydrogenolysis of Cbz-D-Glu(D-Trp-OCH₂CH₂CF₃)—O-Bzl (2.0 g, 3.1 mmol) with 1 g of wet 10% Pd—C in EtOH (150 mL) under 45 psi hydrogen pressure in a Parr apparatus for 1.5 h afforded the title compound H-D-Glu(D-Trp-OCH₂CH₂CF₃)—OH (Apo877, 1.2 g) as a white solid after work up and purification. Yield=91%; ¹H NMR (DMSO-D₆+D₂O, 400 MHz) δ ppm: 7.47 (d, J=8.1 Hz, 1H), 7.35 (d, J=8.1 Hz, 1H), 7.18 (s, 1H), 7.08 (t, J=7.1 Hz, 1H), 6.95-7.04 (m, 1H), 4.43 (dd, J=9.1, 5.1 Hz, 1H), 4.21 (qt, J=11.7, 5.7 Hz, 2H), 3.30 (t, J=6.6 Hz, 1H), 3.10-3.21 (m, 1H), 2.96-3.08 (m, 1H), 2.41-2.64 (m, 2H), 2.17-2.36 (m, 2H), 1.76-1.96 (m, 2H); MS-ESI (m/z): 430 [M+1]⁺.

### Example 16

### Preparation of H-D-Glu(L-Trp-OCH₂—CO—N(CH₃)₂)—OH, Apo894

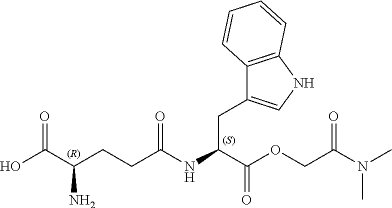

Cbz-D-Glu(OH)—OBzl (18.57 g, 50.0 mmol), HOSu (6.04 g, 52.5 mmol) and EDCl hydrochloride (10.55 g, 55.0 mmol) were mixed in DMF (75 mL) and stirred for 2.5 h. L-Trp-OH (12.25 g, 55.0 mmol) was then added to the reaction mixture. After stirring at RT for overnight, the mixture was diluted with ethyl acetate, then washed with a 0.5N HCl solution (×2), water and brine, dried over MgSO4 and filtered. The filtrate was concentrated by rotary evaporation to give Cbz-D-Glu(L-Trp-OH)—OBzl (27.5 g) as a white solid. Yield=98%. ¹H NMR (DMSO-D₆, 400 MHz) d ppm: 12.55 (br. s, 1H), 10.83 (br. s, 1H), 8.14 (d, J=8.1 Hz, 1H), 7.78 (d, J=8.1 Hz, 1H), 7.51 (d. J=7.1 Hz, 1H), 7.28-7.44 (m, 11H), 7.12 (s, 1H), 7.02-7.10 (m, 1H), 6.90-7.01 (m, 1H), 5.12 (s, 2H), 4.97-5.08 (m, 2H), 4.35-4.50 (m, 1H), 4.04-4.15 (m, 1H), 3.14 (dd, J=14.7, 4.5 Hz, 1H), 2.98 (dd, J=14.7, 8.6 Hz, 1H), 2.12-2.27 (m, 2H), 1.87-2.00 (m, 1H), 1.64-1.81 (m, 1H).

To a mixture of Cbz-D-Glu(L-Trp-OH)—OBzl (2.24 g, 4.08 mmol) with potassium carbonate (1.11 g, 8.0 mmol) in N,N-dimethylformamide (20 mL) warmed under a 45° C. temperature oil bath was added 2-chloro-N,N-dimethylacetamide (0.73 g, 6.0 mmol). After stirring for 3 h, the reaction mixture was diluted with water and extracted with ethyl acetate. The organic layer was washed with water (×3) then brine. The product was purified by column chromatography on silica gel using a solvent mixture of ethyl acetate/hexanes (8/2, v/v) to give the desired alkylated compound Cbz-D-Glu(L-Trp-OCH₂—CO—N(CH₃)₂)—OBzl (1.79 g) as a white foam. Yield=69%; ¹H NMR (DMSO-D₆, 300 MHz) δ ppm: 10.85 (br. s, 1H), 8.34 (d, J=7.5 Hz, 1H), 7.78 (d, J=7.5 Hz, 1H), 7.50 (d, J=7.5 Hz, 1H), 7.34 (br. s, 11H), 7.19 (s, 1H), 7.03-7.13 (m, 1H), 6.93-7.02 (m, 5.12 (s, 2H), 4.96-5.09 (m, 2H), 4.81 (q, J=15.1 Hz, 2H), 4.49-4.62 (m, 1H), 4.02-4.15 (m, 1H), 3.27-3.33 (m, 1H), 3.01 (dd, J=14.3, 9.8 Hz, 1H), 2.90 (s, 3H), 2.83 (s, 3H), 2.12-2.30 (m, 2H), 1.87-1.98 (m, 1H), 1.64-1.80 (m, 1H); MS (m/z): 643 [M+1]⁺.

Cbz-D-Glu(L-Trp-OCH₂—CO—N(CH₃)₂)—OBzl (1.65 g, 2.6 mmol) and 10% Pd—C(wet, 0.36 g) was mixed in ethanol (100 mL). The reaction mixture was hydrogenated in a Parr apparatus for 1.5 h under an atmosphere of hydrogen. The mixture was filtered through Celite™. The filtrate was concentrated by rotary evaporation under reduced pressure and the residue was triturated with acetonitrile. The title compound H-D-Glu(L-Trp-OCH₂—CO—N(CH₃)₂)—OH (Apo894, 1.00 g) was collected by suction filtration as a white solid. Yield=92%; ¹H NMR (DMSO-D₆+D₂O, 300 MHz) d ppm: 7.49 (d, J=7.5 Hz, 1H), 7.34 (d, J=7.5 Hz, 1H), 7.18 (br. s, 1H), 6.90-7.12 (m, 2H), 4.79 (q, J=15.1 Hz, 2H), 4.47-4.61 (m, 1H), 3.26-3.39 (m, 1H), 3.19 (t, J=5.7 Hz, 1H), 2.94-3.12 (m, 1H), 2.88 (br. s, 3H), 2.81 (br. s, 3H), 2.11-2.33 (m, 2H), 1.68-1.93 (m, 2H).

### Example 17

### Preparation of H-D-Glu(D-Trp-O-mofetil)-O-mofetil.3HCl, Apo903.3HCl

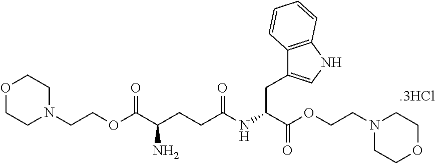 [SMILES: N[C@H](CCC(=O)N[C@H](Cc1c[nH]c2ccccc12)C(=O)OCCN1CCOCC1)C(=O)OCCN1CCOCC1]

### Preparation of Boc-D-Glu(D-Trp-O-mofetil)-O-mofetil

To a solution of 2-morpholinoethanol (3.94 g, 30.0 mmol) with triethylamine (5.06 g, 50 mmol) in dichloromethane (40 mL) cooled in an ice-water bath, methanesulfonyl chloride (3.44 g, 30.0 mmol) was carefully added. After stirring for 10 min, the reaction mixture was concentrated under reduced pressure by rotary evaporation. The residue was mixed with potassium carbonate (4.15 g, 30.0 mmol) and Boc-D-Glu(D-Trp-OH)—OH (4.33 g, 10.0 mmol) in DMF (30 mL) with ice-water bath cooling. The mixture was then heated to 40° C. and stirred for overnight. The mixture was allowed to cool to RT and diluted with ethyl acetate. The inorganic salt was removed by suction filtration and the filtrate was washed with water (×3) and brine. The ethyl acetate layer was concentrated with silica gel and the crude mixture was purified by column chromatography with a solvent mixture of acetone and ethyl acetate (gradient, 1/9 to 4/6 ratio, v/v) to give Boc-D-Glu(D-Trp-O-mofetil)-O-mofetil (3.13 g) as a white foam. Yield=47%; ¹H NMR (DMSO-D₆, 400 MHz) δ ppm: 10.86 (br. s, 1H), 8.26 (d, J=7.1 Hz, 1H), 7.47 (d, J=8.1 Hz, 1H), 7.33 (d, J=8.1 Hz, 1H), 7.25 (d, J=8.1 Hz, 1H), 7.15 (s, 1H), 7.06 (t, J=7.1 Hz, 1H), 6.95-7.01 (m, 1H), 4.46 (q, J=7.1 Hz, 1H), 4.17-4.27 (m, 1H), 3.97-4.12 (m, 3H), 3.88-3.97 (m, 1H), 3.43-3.57 (m, 8H), 3.13 (dd, J=14.7, 6.6 Hz, 1H), 3.01 (dd, J=14.7, 7.6 Hz, 1H), 2.20-2.55 (m, 14H), 1.80-1.94 (m, 1H), 1.65-1.78 (m, 1H), 1.38 (s, 9H); MS-ESI (m/z): 660 [M+1]⁺.

Proceeding in a similar manner as described under example 6B, the title compound H-D-Glu(D-Trp-O-mofetil)-O-mofetil.3HCl (Apo903.3HCl, 590 mg, yield=88%) was obtained from deprotection of Boc-D-Glu(D-Trp-O-mofetil)-O-mofetil (660 mg, to mmol) in 4M HCl in dioxane (4 mL) and ethyl acetate (20 mL); ¹H NMR (DMSO-D₆, 400 MHz) δ ppm: 11.21 (br. s, 2H), 10.94 (br. s, 1H), 8.70 (m, 4H), 7.51 (d, J=7.1 Hz, 1H), 7.35 (d, J=8.1 Hz, 1H), 7.21 (s, 1H), 7.08 (t, J=7.1 Hz, 1H), 6.95-7.04 (m, 1H), 4.28-4.62 (m, 5H), 4.02-4.13 (m, 1H), 3.71-3.99 (m, 9H), 2.85-3.47 (m, 13H), 2.29-2.44 (m, 2H), 1.98-2.06 (m, 2H); MS MS-ESI (m/z): 560 [M+1]⁺ (free base).

### Example 18

### Preparation of H-D-Glu(D-Trp-O—CH₂CH₂CF₃)—O—CH₂CH₂CF₃ hydrochloride (Apo879.HCl)

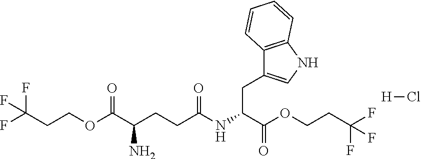

To a suspension of H-D-Glu(D-Trp-OH)—OH (2.0 g, 6.0 mmol) in 3,3,3-trifluoropropan-1-ol (8.5 mL, 96.4 mmol) was bubbled HCl (gas) at ice-water bath temperature. The resulting mixture was allowed to warm to RT and then stirred for overnight. The reaction mixture was concentrated to dryness in vacuo. The residue was purified by flash column chromatography on silica gel using a solvent mixture of IPA and CH₂Cl₂ (from 119 to 2/8, v/v) as eluent. Fractions rich in product were pooled together and concentrated in vacuo. The residue was stirred in 2M HCl in Et₂O (10 mL), then concentrated to dryness and dried under vacuum to afford the title compound (1.8 g). Y=53.4%; ¹H NMR (DMSO-D₆, 400 MHz) δ ppm: 11.00 (br. s, 1H), 8.73 (br. s, 3H), 8.58 (d, J=7.1 Hz, 1H), 7.48 (d, J=8.1 Hz, 1H), 7.36 (d, J=8.1 Hz, 1H), 7.22 (s, 1H), 7.04-7.13 (m, 1H), 6.96-7.04 (m, 1H), 4.43-4.53 (m, 1H), 4.29-4.43 (m, 2H), 4.14-4.29 (m, 2H), 3.95 (t, J=6.1 Hz; 1H), 3.12-3.24 (m, 1H), 3.01-3.12 (m, 1H), 2.64-2.81 (m, 2H), 2.47-2.64 (m, 2H), 2.23-2.45 (m, 2H), 1.91-2.06 (m, 2H); MS-ESI (m/z): 526 [M+1]⁺.

### Example 19

### Preparation of H-D-Glu(D-Trp-O—CH(CH₃)—O—CO—O-cyclohexyl)-O-Et hydrochloride salt, Apo854.HCl

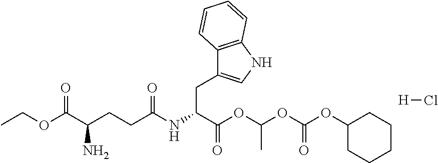

Cbz-D-Glu(OH)—O-Et (12.1 g, 39.1 mmol), HOSu (4.60 g, 40.0 mmol) and EDCl.HCl (7.67 g, 40.0 mmol) were mixed in DMF (100 mL) under ice-water bath temperature. The reaction mixture was allowed to warm to RT then stirred for overnight. The reaction mixture was cooled again in an ice-water bath and D-Trp-OH (8.17 g, 40.0 mmol) was added. The mixture was stirred at room temperature for overnight. The mixture was poured into a beaker containing 0.5N HCl (200 mL) and ice pellets. The mixture was extracted with ethyl acetate (2×200 mL+1×100 mL). The organic layers were combined and washed with a 0.5N HCl solution (100 mL), water (2×100 mL) and brine (100 mL), dried over MgSO4, then filtered. The filtrate was concentrated via rotary evaporation under reduced pressure and the resulting solid Cbz-D-Glu(D-Trp-OH)—O-Et was triturated with 10% ethyl acetate in hexanes. The precipitated white solid was collected via suction filtration (17.6 g). Yield=90%; ¹H NMR (DMSO-D₆, 400 MHz) δ ppm: 12.58 (br. s, 1H), 10.82 (s, 1H), 8.12 (d, J=8.1 Hz, 1H), 7.71 (d, J=8.1 Hz, 1H), 7.52 (d, J=8.1 Hz, 1H), 7.23-7.42 (m, 6H), 7.12 (s, 1H), 7.06 (t, J=7.6 Hz, 1H), 6.97 (t, J=7.6 Hz, 1H), 4.97-5.10 (m, 2H), 4.41-4.51 (m, 1H), 3.95-4.15 (m, 3H), 3.15 (dd. J=14.1, 5.1 Hz, 1H), 2.99 (dd, J=15.2, 8.1 Hz, 1H), 2.09-2.26 (m, 2H), 1.83-1.96 (m, 1H), 1.65-1.81 (m, 1H), 1.16 (t, J=7.1 Hz, 3H); MS-ESI (m/z): 496 [M+1]⁺.

To a mixture of Cbz-D-Glu(D-Trp-OH)—O-Et (4.95 g, 10.0 mmol) with potassium carbonate (4.15 g, 30.0 mmol) and sodium iodide (6.00 g, 40.0 mmol) in N,N-dimethylformamide (30 mL) at room temperature, 1-chloroethylcyclohexyl carbonate (6.20 g, 30.0 mmol) was added, After being stirred at room temperature for overnight, additional N,N-dimethylformamide (30 mL) was added and the reaction mixture was stirred at 40° C. for overnight. The reaction mixture was diluted with ethyl acetate then washed with water (3×) then with brine. The crude product Cbz-D-Glu(D-Trp-O—CH(CH₃)—O—CO—O-cyclohexyl)-O-Et was purified by column chromatography on silica gel using a solvent gradient of a mixture of ethyl acetate in hexanes (20 to 40%) as eluant. Fractions rich in product were combined together and evaporated to dryness. Thus, the desired compound Cbz-D-Glu(D-Trp-O—CH(CH₃)—O—CO—O-cyclohexyl)-O-Et (4.43 g) was obtained as a pale-yellow foam. Yield=66° A); ¹H NMR (DMSO-D₆, 400 MHz) δ ppm: 10.86 (br. s, 1H), 8.36 (dd, J=17.2, 7.1 Hz, 1H), 7.66-7.77 (m, 1H), 7.46 (t, J=8.0 Hz., 1H), 7.22-7.42 (m, 6H), 7.10-7.20 (m, 1H), 7.02-7.10 (m, 1H), 6.90-7.02 (m, 1H), 6.58-6.70 (m, 0.5H), 6.46-6.58 (m, 0.5H), 5.04 (br. s, 2H), 4.38-4.61 (m, 2H), 3.93-4.15 (m, 3H), 2.90-3.17 (m, 2H), 2.20 (br. s, 2H), 1.54-1.96 (m, 6H), 1.02-1.53 (m, 12H); MS-ESI (m/z): 666 [M+1]⁺.

Cbz-D-Glu(D-Trp-O—CH(CH₃)—O—CO—O-cyclohexyl)-O-Et (2.0 g, 3.0 mmol) and 10% Pd/C (wet, 0.6 g) was mixed in ethanol (50 mL) and 2M HCl in ether (1.7 mL, 3.4 mmol). The reaction mixture was hydrogenated in a Parr apparatus at 20-25 psi of hydrogen pressure for an hour. The mixture was filtered through Celite™ and the cake was washed with ethanol. The filtrate was concentrated by rotary evaporation and the residue was triturated with a mixture of ether and hexanes. Thus, H-D-Glu(D-Trp-O—CH(CH₃)—O—CO—O-cyclohexyl)-O-Et hydrochloride salt (Apo854.HCl, 0.80 g) was obtained as a pink solid foam. Yield=47%; ¹H NMR (DMSO-D₆, 400 MHz) δ ppm: 10.94 (br. s, 1H), 8.57 (br. s, 4H), 7.47 (t, J=8.1 Hz, 1H), 7.34 (d, J=8.1 Hz, 1H), 7.19 (s, 1H), 7.07 (t, J=7.6 Hz, 1H), 6.88-7.03 (m. 1H), 6.58-6.72 (q, J=5.1 Hz, 0.5H), 6.53 (q, J=5.1 Hz, 0.5H), 4.39-4.63 (m, 2H), 4.00-4.26 (m, 2H), 3.78-4.00 (m, 1H), 2.93-3.18 (m, 2H), 2.18-2.41 (m, 2H), 1.88-2.02 (m, 2H), 1.82 (br. s, 2H), 1.63 (br. s, 2H), 1.13-1.53 (m, 12H); MS-ESI (m/z): 532 [M+1]⁺ (free base).

### Example 20

### Preparation of H-D-Glu(D-Trp-O—CH(CH₃)—O—CO—O-Et)-O-Et hydrochloride, Apo901.HCl

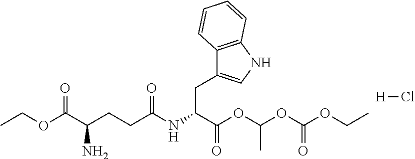

Proceeding in a similar manner as described in Example 19 above, Cbz-D-Glu(D-Trp-O—CH(CH₃)—O—CO—O-Et)-O-Et (1.64 g, yield 53%) was prepared from the reaction of CBz-D-Glu(D-Trp-OH)—O-Et (2.48 g, 5.00 mmol) with 1-chloroethyl ethyl carbonate (1.53 g, 10.0 mmol) in presence of potassium carbonate (1.38 g, 10.0 mmol) and sodium iodide (3.00 g, 20.0 mmol) in N,N-dimethylformamide (30 mL) at 50° C. overnight. ¹H NMR (DMSO-D₆, 400 MHz) δ ppm: 10.87 (br. s, 1H), 8.24-8.48 (m, 1H), 7.72 (t, J=7.1 Hz, 1H), 7.42-7.55 (m, 1H), 7.22-7.42 (m, 6H), 7.14 (d, J=5.1 Hz, 1H), 7.07 (t, J=7.6 Hz, 1H), 6.91-7.02 (m, 1H), 6.63 (q, J=5.1 Hz, 0.5H), 6.51 (q, J=5.1 Hz, 0.5H), 4.97-5.13 (m, 2H), 4.37-4.51 (m, 1H), 3.88-4.23 (m, 5H), 2.92-3.20 (m, 2H), 2.10-2.28 (m, 2H), 1.80-1.96 (m, 1H), 1.73 (m, 1H), 1.43 (d, J=5.1 Hz, 1.5H), 1.12-1.29 (m, 7.5H).

H-D-Glu(D-Trp-O—CH(CH₃)—O—CO—O-Et)-O-Et hydrochloride (Apo901.HCl, 0.97 g) was obtained from the hydrogenation of Cbz-D-Glu(D-Trp-O—CH(CH₃)—O—COO-Et)-O-Et (1.60, 2.60 mmol) with 10% Pd/C (wet, 1.00 g) in ethanol (75 mL) and 4M HCl in dioxane (0.8 mL) in a Parr apparatus under a hydrogen atmosphere. Yield=72%; ¹H NMR (DMSO-D₆, 400 MHz) δ ppm: 11.04 (br. s, 1H), 8.56-8.89 (m, 4H), 7.42-7.53 (m, 1H), 7.35 (d, J=7.1 Hz, 1H), 7.22 (s, 1H), 7.06 (t, J=7.1 Hz, 1H), 6.91-7.01 (m, 1H), 6.64 (m, 0.5H), 6.54 (m, 0.5H), 4.39-4.57 (m, 1H), 4.05-4.27 (m, 4H), 3.80-3.97 (m, 1H), 2.97-3.24 (m, 2H), 2.20-2.45 (m, 2H), 1.92-2.07 (m, 2H), 1.45 (d, J=5.1 Hz, 1.5H), 1.12-1.30 (m, 7.5H); MS-ESI (m/z): 478 [M+1]⁺ (free base).

### Example 21

### Preparation of H-D-Glu(D-Trp-O-mofetil)-O-Et.2HCl, Apo900.2HCl

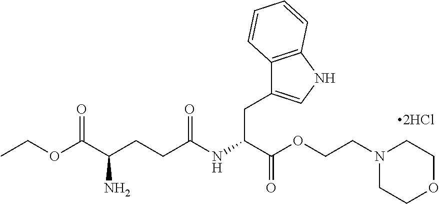 [SMILES: CCOC(=O)[C@H](N)CCC(=O)N[C@H](Cc1c[nH]c2ccccc12)C(=O)OCCN1CCOCC1]

Proceeding in a similar manner as described in Example 19 above, Cbz-D-Glu(D-Trp-O-mofetil)-O-Et hydrochloride salt (2.21 g, yield 34%) was prepared from the reaction of Cbz-D-Glu(D-Trp-OH)—O-Et (4.96 g, 10.0 mmol) with 2-morpholinoethyl methanesulfonate, which was made from 2-morpholinoethanol (1.97 g, 15.0 mmol) with methanesulfonyl chloride (1.72 g, 15.0 mmol), in presence of potassium carbonate (2.76 g, 20.0 mmol) in N,N-dimethylformamide (30 mL). ¹H NMR (DMSO-D₆, 400 MHz) δ ppm: 11.07 (br. s, 1H), 10.90 (br. s, 1H), 8.48 (d, J=7.1 Hz, 1H), 7.73 (d, J=8.1 Hz, 1H), 7.50 (d, J=7.1 Hz, 1H), 7.27-7.43 (m, 611), 7.17 (s, 1H), 7.07 (t, J=7.6 Hz, 1H), 6.93-7.04 (m, 1H), 4.98-5.11 (m, 2H), 4.54 (q, J=7.1 Hz, 1H), 4.25-4.44 (m, 2H), 3.96-4.14 (m, 3H), 3.67-3.91 (m, 4H), 3.04-3.33 (m, 6H), 2.85-3.04 (m, 2H), 2.19-2.30 (m, 2H), 1.85-1.97 (m, 1H), 1.68-1.82 (m, 1H), 1.17 (t, J=7.1 Hz, 3H); MS-ESI (m/z): 609 [M+1]⁺ (free base).

H-D-Glu(D-Trp-O-mofetil)-O-Et dihydrochloride salt (1.22 g, 65%) was prepared from the hydrogenation of Cbz-D-Glu(D-Trp-O-mofetil)-O-Et hydrochloride (2.21, 3.40 mmol) with 10% Pd/C (wet, 1.4 g) in ethanol (100 mL) and 2M HCl in ether (2.5 mL) in a Parr apparatus under a hydrogen atmosphere. ¹H NMR (DMSO-D₆, 400 MHz) δ ppm: 11.80 (br. s, 1H), 11.02 (br. s, 1H), 8.66-8.85 (m. 4H), 7.51 (d, J=8.1 Hz, 1H), 7.35 (d, J=8.1 Hz, 1H), 7.23 (br. s, 1H), 7.06 (t, J=7.1 Hz, 1H), 6.94-7.02 (m, 1H), 4.56 (q, J=7.1 Hz, 1H), 4.33-4.45 (m, 2H), 4.06-4.23 (m, 2H). 3.86 (br. s, 5H), 2.90-3.37 (m, 8H), 2.26-2.46 (m, 2H), 1.91-2.03 (m, 2H), 1.20 (t, J=6.6 Hz, 3H); MS-ESI (m/z): 475 [M+1]⁺ (free base).

### Example 22

### Preparation of H-D-Glu(D-Trp-O-5-indanyl)-OH, Apo851

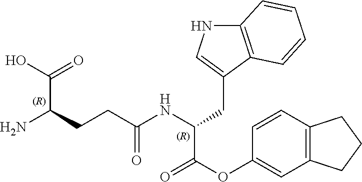

### A. Preparation of H-D-Trp-O-5-indanyl hydrochloride

Boc-D-Trp-OH (3.04 g, 10.0 mmol), 5-indanol (5.41 g, 40.0 mmol), EDCl.HCl (2.30 g, 12.0 mmol), HOBt hydrate (1.68 g, 11.0 mmol) and N-methylmorpholine (1.21 g, 12.0 mmol) were mixed in dichloromethane (10 mL). The reaction mixture was stirred at room temperature for overnight and then diluted with ethyl acetate. The mixture was washed with water (2×) and brine, then dried over magnesium sulphate. The product was purified by column chromatography on silica gel using a solvent gradient consisting of a mixture of ethyl acetate (5 to 20%) in hexanes as eluent to give Boc-D-Trp-O-5-indanyl (3.26 g) as a colorless foam. Yield: 77%; NMR (CD₃OD, 90 MHz) δ ppm: 7.60 (d, J=7.0 Hz, 1H), 7.27-7.47 (m, 1H), 6.88-7.27 (m, 4H), 6.48-6.82 (m, 2H), 4.63 (t, J=6.9 Hz, 1H) 4.10 (q, 6.8 Hz, 1H) 2.63-3.05 (m, 4H), 1.87-2.31 (m, 3H) 1.09-1.65 (m, 11H); MS-ESI (m/z) 421 [M+1]⁺.

Boc-D-Trp-O-5-indanyl (3.25 g, 7.70 mmol) was mixed with 2M HCl in ether (20 mL) at room temperature and stirred for 20 h. Additional 2M HCl in ether (10 mL) was added and the mixture was kept stirring for another 3.5 h. The precipitate was collected by suction filtration, thoroughly washed with ether to give H-D-Trp-O-5-indanyl hydrochloride as off-white solid (2.01 g). Yield: 72%; ¹H NMR (DMSO-D₆, 400 MHz) δ ppm: 7.57 (d, J=8.1 Hz, 1H), 7.40 (d, J=8.1 Hz, 1H), 7.31 (s, 1H), 7.08-7.20 (m, 2H), 6.97-7.06 (m, 1H), 6.51-6.62 (m, 2H), 4.45 (t, J=6.6 Hz, 1H), 3.30-3.49 (m, 2H), 2.70-2.84 (m, 4H), 1.91-2.05 (m, 2H); MS m/z: 321 [M+1]⁺ (free base).

### B. Preparation of Cbz-D-Glu(D-Trp-O-5-indanyl)-O-Bzl

H-D-Trp-O-5-indanyl hydrochloride (1.00 g, 2.8 mmol), Cbz-D-Glu-O-Bzl (1.04 g, 2.80 mmol), EDCl.HCl (0.64 g, 3.30 mmol), HOBt hydrate (0.47 g, 3.10 mmol) and N-methylmorpholine (0.57 g, 5.60 mmol) were mixed in dichloromethane (10 mL). The reaction mixture was stirred at room temperature for overnight and then diluted with ethyl acetate. The mixture was washed with water, a saturated sodium bicarbonate solution, water, 0.5N HCl solution and brine, then dried with magnesium sulphate. The organic solution was concentrated by rotary evaporation and the residue was triturated with ether to give Cbz-D-Glu(D-Trp-O-5-indanyl)-O-Bzl (1.63 g) as a white solid. Yield 87%; ¹H NMR (DMSO-D₆, 400 MHz) δ ppm: 10.92 (br. s, 1H), 8.52 (d, J=6.1 Hz, 1H), 7.82 (d, J=8.1 Hz, 1H), 7.54 (d, J=7.1 Hz, 1H), 7.19-7.41 (m, 12H), 7.04-7.18 (m, 2H), 6.94-7.04 (m, 1H), 6.60 (s, 1H), 6.57 (d, J=8.1 Hz, 1H), 5.13 (s, 2H), 4.99-5.09 (m, 2H), 4.55-4.70 (m, 1H), 4.08-4.21 (m, 1H), 3.12-3.28 (m, 2H), 2.70-2.86 (m, 4H), 2.29 (br. s, 2H), 1.92-2.08 (m, 3H), 1.69-1.90 (m, 1H); MS-ESI (m/z): 674 [M+1]⁺.

### C. Preparation of H-D-Glu(D-Trp-O-5-indanyl)-OH, Apo851

Cbz-D-Glu(D-Trp-O-5-indanyl)-O-Bzl (1.62 g, 2.4 mmol) and 10% Pd/C (wet, 0.60 g) were mixed in ethanol (180 mL). The reaction mixture was hydrogenated under a hydrogen atmosphere using a balloon for 4 h. The mixture was filtered through Celite™ and the cake was washed with ethanol. The filtrate was concentrated by rotary evaporation and the residue was triturated with acetonitrile to give H-D-Glu(D-Trp-O-5-indanyl)-OH (Apo851, 0.95 g) as a white solid. Yield=80%; ¹H NMR (DMSO-D₅, 400 MHz) δ ppm: 10.99 (br. s, 1H), 9.02 (d, J=7.1 Hz, 1H), 7.55 (d, J=8.1 Hz, 1H), 7.37 (d, J=8.1 Hz, 1H), 7.28 (s, 1H), 7.15 (d, J=8.1 Hz, 1H), 7.04-7.12 (m, 1H), 6.92-7.04 (m, 1H), 6.64 (s, 1H), 6.59 (d, J=8.1 Hz, 1H), 4.60 (q, J=7.1 Hz, 1H), 3.11-3.43 (m, 4H), 2.71-2.87 (m, 4H), 2.23-2.42 (m, 2H), 1.94-2.11 (m, 2H), 1.71-1.93 (m, 2H); MS-ESI (m/z): 450 [M+1]⁺.

### Example 23

### Preparation of H-D-Glu(D-Trp-O-(2-methoxyphenyl))-OH, Apo852

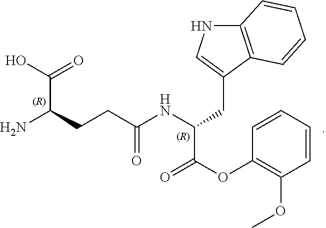

### A. Preparation of H-D-Trp-O-(2-methoxyphenyl) hydrochloride

Proceeding in a similar manner as described in Example 22A above, Boc-D-Trp-O-(2-methoxyphenyl) (5.85 g, yield=70%) was prepared from Boc-D-Trp-OH (6.08 g, 20.0 mmol), EDCl.HCl (4.60 g, 24.0 mmol), HOBt hydrate (3.36 g, 22.0 mmol), N-methylmorpholine (2.42 g, 24.0 mmol) and 2-methoxyphenol (10.3 g, 80.0 mmol) in dichloromethane (20 mL) at room temperature. ¹H NMR (DMSO-D₆, 400 MHz) δ ppm: 10.89 (br. s, 1H), 7.56 (d, J=8.1 Hz, 1H), 7.42 (d, J=8.1 Hz, 1H), 7.36 (d, J=8.1 Hz, 1H), 7.20-7.31 (m, 2H), 7.11-7.16 (m, 1H), 7.09 (t, J=7.6 Hz, 1H), 6.92-7.03 (m, 3H), 4.36-4.51 (m, 1H), 3.76 (s, 3H), 3.31-3.38 (m, 1H), 3.09-3.20 (m, 1H), 1.35 (s, 7.5H), 1.28 (s, 1.5H).

H-D-Trp-O-(2-methoxyphenyl) hydrochloride (4.55 g, yield=96%) was prepared from the reaction of Boc-D-Trp-O-(2-methoxyphenyl) (5.64 g, 13.6 mmol) with 2M HCl in ether (40 mL) at room temperature.

### B. Preparation of Cbz-D-Glu(D-Trp-O-(2-methoxyphenyl))-O-Bzl

Proceeding in a similar manner as described in Example 22B above, Cbz-D-Glu(D-Trp-O-(2-methoxyphenyl))-O-Bzl (2.25 g, yield=47%) was prepared from H-D-Trp-O-(2-methoxyphenyl) hydrochloride (2.50 g, 7.2 mmol), EDCl.HCl (1.66 g, 8.6 mmol), HOBt hydrate (1.21 g, 7.9 mmol), N-methylmorpholine (1.53 g, 15.1 mmol) and Cbz-D-Glu-O-Bzl (2.68 g, 7.2 mmol) in dichloromethane (20 mL) at room temperature. ¹H NMR (DMSO-D₆, 400 MHz) δ ppm: 10.90 (br. s, 1H), 8.50 (d, J=8.1 Hz, 1H), 7.81 (d, J=7.1 Hz, 1H), 7.56 (d, J=8.1 Hz, 1H), 7.19-7.40 (m, 13H), 7.05-7.15 (m, 2H), 6.96-7.03 (m, 1H), 6.87-6.96 (m, 2H), 5.12 (s, 2H), 4.98-5.09 (m, 2H), 4.69-4.79 (m. 1H), 4.05-4.16 (m, 1H), 3.73 (s, 3H), 3.32-3.41 (m, 1H), 3.15 (dd, J=14.7, 8.6 Hz, 1H), 2.15-2.35 (m, 2H), 1.88-2.03 (m, 1H), 1.70-1.86 (m, 1H); MS-ESI (m/z): 664 [M+1]⁺.

### C. Preparation of H-D-Glu(D-Trp-O-(2-methoxyphenyl))-OH, Apo852

Proceeding in a similar manner as described in Example 22C above, H-D-Glu(D-Trp-O-(2-methoxyphenyl))-OH, Apo852 (1.11 g, yield=84%) was prepared from the hydrogenation of Cbz-D-Glu(D-Trp-O-(2-methoxyphenyl))-O-Bzl (2.00 g, 3.0 mmol) with 10% Pd/C (wet, 0.75 g) in ethanol (200 mL) under an atmosphere of hydrogen using a balloon. ¹H NMR (CD₃OD, 400 MHz) δ ppm: 7.60 (d, J=8.1 Hz, 1H). 7.35 (d, J=8.1 Hz, 1H), 7.17-726 (m, 2H), 6.99-7.14 (m, 3H), 6.88-6.95 (m, 2H), 4.99 (dd, J=9.1, 5.1 Hz, 1H), 3.78 (s, 3H), 3.60 (t, J=6.1 Hz, 1H), 3.54 (dd, J=14.1, 5.1 Hz, 1H), 3.21-3.30 (m, 1H), 2.35-2.57 (m, 2H), 1.98-2.11 (m, 2H); MS-ESI (m/z): 440 [M+1]⁺.

### Example 24

### Preparation of H-D-Glu(D-Trp-O-mofetil)-O—CH₂CH₂CF₃ dihydrochloride (Apo913.2HCl)

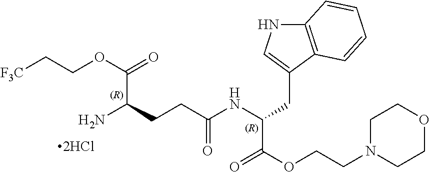

### A. Preparation of Boc-D-Trp-O-mofetil

A solution of Boc-D-Trp-OH (30.4 g, 100.0 mmol), 2-morpholinoethanol (13.2 g, 100.0 mmol), EDCl.HCl (19.2 g, 100.0 mmol), HOBt hydrate (15.3 g, 100.0 mmol) in dichloromethane (300 mL) was stirred at room temperature. After two days the reaction mixture was concentrated in vacuo. The residue was diluted with ethyl acetate. The resulting solution was successively washed with a saturated sodium bicarbonate solution (2×), water (2×) and brine, then dried over magnesium sulphate. After filtration, the organic fraction was concentrated in vacuo to give crude Boc-D-Trp-O-mofetil (37.6 g) as a pale-brown oil. The product was used in the next step reaction without further purification. ¹H NMR (CDCl₃, 400 MHz) δ (ppm): 8.37 (br. s, 1H), 7.56 (d, J=8.1 Hz, 1H), 7.34 (d, J=8.1 Hz, 1H), 7.18 (t, J=7.6 Hz, 1H), 7.06-7.14 (m, 1H), 7.03 (s, 1H), 5.12 (d, J=8.1 Hz, 1H), 4.58-4.71 (m, 1H), 4.10-4.20 (m, 2H), 3.58-3.73 (m, 4H), 3.19-3.35 (m, 2H), 2.43-2.56 (m, 21-1), 2.29-2.42 (m, 4H), 1.43 (s, 9H).

### B. Preparation of H-D-Trp-O-mofetil dihydrochloride

To a solution of Boc-D-Trp-O-mofetil (37.0 g. 89.0 mmol) in ethyl acetate (250 mL) was slowly bubbled HCl gas for 3 h. The resulting precipitate was collected via suction filtration and thoroughly washed with ethyl acetate to give H-D-Trp-O-mofetil dihydrochloride (30.6 g) as an off-white solid. Yield=88%; ¹H NMR (DMSO-D₆, 400 MHz) δ (ppm): 11.35 (br. s, 1H), 11.11 (br. s, 1H), 8.77 (br. s, 3H), 7.56 (d, J=8.1 Hz, 1H), 7.38 (d, J=8.1 Hz, 1H), 7.29 (s, 1H), 7.10 (t, J=7.6 Hz, 1H), 6.93-7.06 (m, 1H), 4.33-4.57 (m, 2H), 4.22-4.31 (m, 1H), 3.85 (br. s, 4H), 2.82-3.48 (m, 8H); MS-ESI (m/z): 318 [M+1]⁺ (free base).

### C. Preparation of Boc-D-Glu-(OBn)-O—CH₂CH₂CF₃

A mixture of Boc-D-Glu-(OBn)-OH (6.75 g, 20.0 mmol), 3,3,3-trifluoropropanol (2.28 g, 20.0 mmol), EDCl.HCl (3.84 g, 20.0 mmol) and HOBt hydrate (3.06 g, 20.0 mmol) in dichloromethane (100 mL) was stirred at room temperature for overnight. The reaction mixture was concentrated in vacuo, and the residue was diluted with ethyl acetate. The resulting solution was successively washed with a 1N HCl solution (2×), a saturated sodium bicarbonate solution (2×), water (2×) and brine, then dried over magnesium sulphate. After filtration, the filtrate was evaporated to dryness and then triturated with ether to afford a first crop of Boc-D-Glu-(OBn)-O—CH₂CH₂CF₃ (3.69 g) as a white solid. The mother liquid was concentrated to give a second crop (1.09 g). Total=4.78 g, ¹H NMR (CDCl₃, 400 MHz) δ (ppm): 7.35 (br. s, 5H), 4.93-5.29 (m, 3H), 4.26-4.50 (m, 2H), 2.34-2.67 (m, 4H), 2.09-2.34 (m, 1H), 1.90-2.04 (m, 1H), 1.35-1.49 (m, 9H).

### D. Preparation of Boc-D-Glu-O—CH₂CH₂CF₃

A mixture of Boc-D-Glu-(OBn)-O—CH₂CH₂CF₃ (4.71 g, 10.8 mmol) and 10% Pd—C(wet, 1.22 g) in ethyl acetate (100 mL) was stirred under a hydrogen atmosphere using a balloon at RT for 2 h. The mixture was filtered through Celite™ and the filtrate was concentrated in vacuo. The residue was triturated with hexanes to give Boc-D-Glu-O—CH₂CH₂CF₃ (3.43 g) as a white solid, which was used without further purification in the next step.

### E. Preparation of Boc-D-Glu-(D-Trp-O-mofetil)-O—CH₂CH₂CF₃

To a mixture H-D-Trp-O-mofetil diHCl (1.26 g, 3.2 mmol), Boc-D-Glu-O—CH₂CH₂CF₃ (1.00 g, 2.94 mmol) and EDCl.HCl (0.62 g, 3.23 mmol) in dichloromethane (75 mL), triethylamine (0.98 g, 9.7 mmol) was added. The reaction mixture was stirred at room temperature for overnight and then concentrated in vacuo. The residue was diluted with ethyl acetate and the resulting solution was successively washed with water, a saturated sodium bicarbonate solution and brine, then dried over magnesium sulphate, filtered, and concentrated with silica gel. The product was purified by column chromatography with ethyl acetate as eluent to give Boc-D-Glu-(D-Trp-O-mofetil)-O—CH₂CH₂CF₃ (0.944 g) as a white foam. Yield=45%. ¹H NMR (CDCl₃, 400 MHz) δ (ppm): 8.21 (br. s, 1H), 7.53 (d, J=7.1 Hz, 1H), 7.35 (d, J=8.1 Hz, 1H), 7.19 (t, J=7.6 Hz, 1H), 7.00-7.15 (m, 2H), 6.37 (br. s, 1H), 5.32 (br. s, 1H), 4.80-5.05 (m, 1H), 4.06-4.48 (m. 5H), 3.58-3.85 (m, 4H), 3.19-3.46 (m, 2H), 2.08-2.69 (m, 10H), 1.80-2.01 (m, 2H), 1.43 (s, 9H); MS-ESI (m/z): 643 [M+1]⁺.

### F. Preparation of H-D-Glu-(D-Trp-O-mofetil)-O—CH₂CH₂CF₃ dihydrochloride

Proceeding In a similar manner as described under example 6B, the title compound H-D-Glu(D-Trp-O-mofetil)-O—CH₂CH₂CF₃.2HCl (Apo913.2HCl, 737 mg, yield=66%) was obtained from the deprotection of Boc-D-Glu(D-Trp-O-mofetil)-O—CH₂CH₂CF₃ (890 mg, 1.4 mmol) in 4M HCl in dioxane (3.45 mL) and ethyl acetate (3.0 mL). ¹H NMR (DMSO-D₅, 400 MHz) δ (ppm): 11.41 (br, s, 1H), 10.95 (br. s, 1H), 8.68 (d, J=7.1 Hz, 1H), 8.61 (br. s, 3H), 7.51 (d, J=8.1 Hz, 1H), 7.35 (d, J=8.1 Hz, 1H), 7.20 (s, 1H), 7.04-7.11 (m, 1H), 6.95-7.03 (m, 1H), 4.57 (q, J=7.1 Hz, 1H), 4.27-4.45 (m, 4H), 3.96-4.07 (m, 1H), 3.85 (br. s, 4H), 2.83-3.37 (m, 8H), 2.63-2.81 (m, 2H), 2.25-2.45 (m, 2H), 1.91-2.06 (m, 2H); MS-ESI (m/z): 543 [(M+1]⁺ (free base).

### Example 25

### Preparation of H-D-Glu(D-Trp-O-mofetil)-O-isoamyl dihydrochloride (Apo917.2HCl)

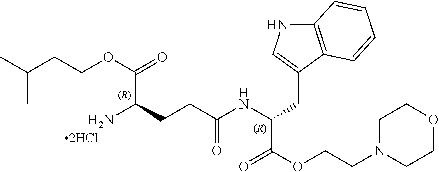

### A. Preparation of Boc-D-Glu(D-Trp-O-mofetil)-O-isoamyl

Proceeding in a similar manner as described in Example 24E above, Boc-D-Glu(D-Trp-O-mofetil)-O-isoamyl (2.22 g, yield=57%) was prepared from the reaction of Boc-D-Glu-O-isoamyl (Example 2, 2.00 g, 6.3 mmol) and H-D-Trp-O-mofetil hydrochloride (Example 24B, 2.46 g, 6.3 mmol) with HOBt hydrate (1.06 g, 6.9 mmol), EDCl hydrochloride (1.38 g, 7.2 mmol) and N-methylmorpholine (0.64 g, 6.3 mmol) in dichloromethane (20 mL) at room temperature for overnight. ¹H NMR (DMSO-D₆, 400 MHz) δ (ppm): 10.86 (br. s, 1H), 8.29 (d, J 7.1 Hz, 1H), 7.47 (d, J=8.1 Hz, 1H), 7.33 (d, J=8.1 Hz, 1H), 7.23 (d, J=7.1 Hz, 1H), 7.15 (s, 1H), 7.07 (t, J=7.6 Hz, 1H), 6.93-7.02 (m, 1H), 4.46 (q, J=7.1 Hz, 1H), 3.96-4.16 (m, 4H), 3.84-3.97 (m, 1H), 3.43-3.57 (m, 4H), 3.08-3.21 (m, 1H), 2.93-3.08 (m, 1H), 2.09-2.45 (m, 8H), 1.79-1.94 (m, 1H), 1.56-1.79 (m, 2H), 1.24-1.55 (m, 11H), 0.87 (d, J=6.1 Hz, 6H); MS-ESI (m/z): 617 [M+1]⁺.

### B. Preparation of H-D-Glu(D-Trp-O-mofetil)-O-isoamyl dihydrochloride

Proceeding In a similar manner as described under example 6B, the title compound H-D-Glu(D-Trp-O-mofetil)-O-isoamyl dihydrochloride (0.81 g, yield=40%) was obtained from the deprotection of Boc-D-Glu(D-Trp-O-mofetil)-O-isoamyl (2.13 g, 1.4 mmol) in 4M HCl in dioxane (15 mL) and ethyl acetate (20 ml). ¹H NMR (DMSO-D₆, 400 MHz) δ (ppm): 11.75 (br. s, 1H), 11.01 (br. s, 1H), 8.72 (br. s, 4H), 7.52 (d, J=8.1 Hz, 1H), 7.35 (d, J=7.1 Hz, 1H), 7.22 (s, 1H), 7.04-7.12 (m, 1H), 6.92-7.02 (m, 1H), 4.49-4.64 (m, 1H), 4.39 (br. s, 2H), 4.14 (br. s, 2H), 3.70-3.97 (m, 5H), 2.80-3.46 (m, 8H), 2.19-2.46 (m, 2H), 1.87-2.11 (m, 2H), 1.55-1.72 (m, 1H), 1.49 (d, J=7.1 Hz, 2H), 0.87 (d, J=7.1 Hz, 6H); MS-ESI (m/z): 517 [M+1]⁺ (free base).

### Example 26

### Preparation of H-D-Glu(D-Trp-O-Bn)-O-mofetil di hydrochloride (Apo904.2HCl)

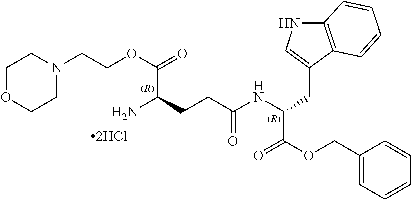

### A. Preparation of Boc-D-Glu-(OBn)-O-mofetil

Proceeding in a similar manner as described in Example 24C above, Boc-D-Glu-(OBn)-O-mofetil (8.70 g, yield=96%) was prepared from the reaction of Boc-D-Glu-(OBn)-OH (6.75 g, 20.0 mmol), 2-morpholinoethanol (2.62 g, 20.0 mmol), HOBt hydrate (3.06 g, 20.0 mmol) and EDCl hydrochloride (3.84 g, 20.0 mmol) in dichloromethane (100 mL) at room temperature for overnight. ¹H NMR (DMSO-D₆, 400 MHz) δ (ppm): 7.16-7.61 (m, 51-1), 5.77 (s, 1H), 5.10 (s, 2H), 4.18-4.42 (m, 1H), 3.88-4.18 (m, 2H), 3.52 (br.s, 4H), 2.51 (br. s, 4H), 2.23-2.46 (m, 4H), 1.71-2.12 (m, 2H), 1.20-1.57 (m, 9H).

### B. Preparation of Boc-D-Glu-(OH)—O-mofetil

Proceeding in a similar manner as described in Example 24D above, Boo-D-Glu-(OH)—O-mofetil (6.58 g, yield=94%) was prepared from the hydrogenation of Boc-D-Glu-(OBn)-O-mofetil (8.70 g, 19.3 mmol) with 10% Pd—C (wet, 2.5 g) in ethyl acetate (100 mL) under a hydrogen atmosphere using a Parr instrument. MS-ESI (m/z): 361 [M+1]+.

### C. Preparation of Boc-D-Glu-(D-Trp-O-Bn)-O-mofetil

Proceeding in a similar manner as described in Example 24E above, Boc-D-Glu-(D-Trp-O-Bn)-O-mofetil (1.72 g, yield=54%) was prepared from the reaction of Boc-D-Glu-O-mofetil (1.80 g, 5.0 mmol) and H-D-Trp-OBn hydrochloride (1.65 g, 5.0 mmol) with EDCl hydrochloride (0.96 g, 5.0 mmol) in dichloromethane (50 mL) at room temperature for overnight. MS-ESI (m/z): 637 [M+1]⁺.

### D. Preparation of H-D-Glu-(D-Trp-O-Bn)-O-mofetil dihydrochloride

Proceeding in a similar manner as described under example 6B, the title compound H-D-Glu-(D-Trp-O-Bn)-O-mofetil dihydrochloride (Apo904.2HCl, 1.26 g, yield=77%) was obtained from the deprotection of Boc-D-Glu-(D-Trp-O-Bn)-0-mofetil (1.70 g, 2.67 mmol) with 4M HCl in dioxane (8 mL) and ethyl acetate (20 mL). ¹H NMR (DMSO-D₆, 400 MHz) δ (ppm): 11.12 (br. s, 1H), 10.91 (s, 1H), 8.66 (br. s, 3H), 8.56 (d, J=7.1 Hz, 1H), 7.48 (d, J=8.1 Hz, 1H), 7.25-7.39 (m, 4H), 7.09-7.20 (m., 3H), 7.08 (t, J=7.6 Hz, 1H), 6.95-7.03 (m, 1H), 4.93-5.10 (m, 2H), 4.38-4.63 (m, 3H), 4.00-4.16 (m, 1H), 3.85-4.00 (m, 4H), 3.00-3.52 (m, 8H), 2.27-2.42 (m, 2H), 1.95-2.16 (m, 2H). MS-ESI (m/z): 537 [M+1]⁺ (free base).

### Example 27

### Preparation of H-D-Glu(D-Trp-OH)—O-mofetil dihydrochloride (Apo905.2HCl)

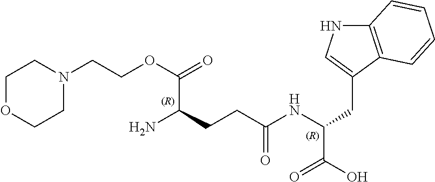

Proceeding in a similar manner as described in Example 22C above, H-D-Glu(D-Trp-OH)—O-mofetil dihydrochloride (Apo905.2HCl, 580 mg, yield=75%) was prepared from the hydrogenation of H-D-Glu-(D-Trp-O-Bn)-O-mofetil dihydrochloride (900 mg, 1.48 mmol) with 10% Pd/C (wet, 450 mg) in ethanol (50 mL) under an atmosphere of hydrogen using a balloon. ¹H NMR (DMSO-D₆, 400 MHz) δ (ppm): 11.28 (br. s, 1H), 10.89 (br. s, 1H), 8.73 (br. s, 3H), 8.34 (d, J=8.1 Hz, 1H), 7.52 (d, J=8.1 Hz, 1H), 7.34 (d, J=8.1 Hz, 1H), 7.17 (s, 1H), 7.06 (t, J=7.6 Hz, 1H), 6.90-7.01 (m, 1H), 4.38-4.63 (m, 3H), 4.03 (br.s, 1H), 3.92 (br. s., 4H), 3.07-3.60 (m, 7H), 3.01 (dd, J=14.7, 8.6 Hz, 1H), 2.27-2.39 (m, 2H), 1.91-2.06 (m, 2H); MS-ESI (m/z): 447 [M+1]⁺.

### Example 28

### Preparation of H-D-Glu(D-Trp-O-5-indanyl)-O-mofetil dihydrochloride (Apo906.2HCl)

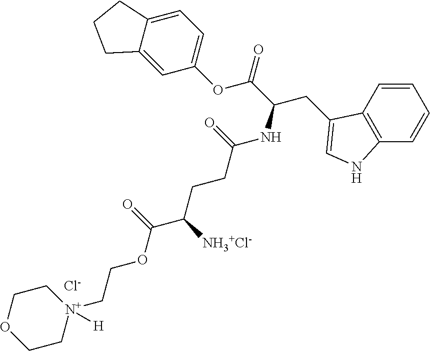

### Preparation of Boc-D-Glu(D-Trp-O-5-indanyl)-O-mofetil

Proceeding in a similar manner as described in Example 24E above, Boc-D-Glu(D-Trp-O-5-indanyl)-O-mofetil (499 mg, yield=75%) was prepared from the reaction of Boc-D-Glu-O-mofetil (2.00 g, 6.3 mmol), H-D-Trp-O-5-indanyl hydrochloride (Example 22A, 2.46 g, 6.3 mmol) with HOBt hydrate (1.06 g, 6.9 mmol), EDCl hydrochloride (1.38 g, 7.2 mmol) and N-methylmorpholine (0.64 g, 6.3 mmol) in dichloromethane (20 mL) at room temperature for overnight. ¹H NMR (CDCl₃, 400 MHz) δ (ppm): 8.79 (br. s, 1H), 7.61 (d, J=8.1 Hz, 1H), 7.35 (d, J=8.1 Hz, 1H), 7.08-7.24 (m, 4H), 6.81 (s, 1H), 6.70-6.77 (m, 2H), 5.14-5.26 (m, 2H), 4.17-4.32 (m, 2H), 4.04-4.14 (m, 1H), 3.63-3.74 (m, 4H), 3.42-3.57 (m, 2H), 2.88 (t, J=7.1 Hz, 4H), 2.61 (t, J=5.1 Hz, 2H), 2.51 (br. s, 4H), 2.24-2.32 (m, 2H), 2.03-2.18 (m, 3H), 1.81-1.96 (m, 1H), 1.44 (br. s, 9H); MS-ESI (m/z): 663 [M+1]⁺.

### Preparation of H-D-Glu(D-Trp-O-5-indanyl)-O-mofetil dihydrochloride

Proceeding in a similar manner as described under Example 6B, the title compound H-D-Glu(D-Trp-O-5-indanyl)-O-mofetil dihydrochloride (Apo906.2HCl, 85 mg, yield=62%) was obtained from the deprotection of Boc-D-Glu(D-Trp-O-5-indanyl)-O-mofetil (142 mg, 0.214 mmol) in 4M HCl in dioxane solution (3 mL) and ethyl acetate (3 mL). ¹H NMR (DMSO-D₆, 400 MHz) δ (ppm): 11.05 (br. s, 1H), 10.97 (br. s, 1H), 8.51-8.91 (m, 4H), 7.54 (d, J=8.1 Hz, 1H), 7.37 (d, J=8.1 Hz, 1H), 7.28 (s, 1H), 7.16 (d, J=8.1 Hz, 1H), 7.09 (t, J=7.6 Hz, 1H), 6.95-7.05 (m. 1H), 6.55-6.67 (m, 2H), 4.38-4.73 (m, 3H), 4.01-4.15 (m, 1H), 3.76-4.01 (m, 4H), 2.98-3.50 (m, 8H), 2.79 (m, 4H), 2.29-2.48 (m, 2H), 1.88-2.17 (m, 4H); MS-ESI (m/z): 563 [M+1]⁺ (free base).

### Example 29

### Preparation of H-D-Glu(D-Trp-O-cyclohexyl)-OH (Apo850)

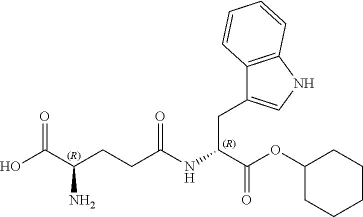

### A. Preparation of H-D-Trp-O-cyclohexyl hydrochloride salt

To a suspension of Boc-D-Trp-OH (15.0 g, 49.4 mmol) in CH₂Cl₂ was added EDC. HCl (14.2 g, 74.1 mmol) at RT. To the resulting clear solution was added cyclohexanol (26.1 mL, 247 mmol) followed by DMAP (0.6 g, 4.9 mmol) at RT. The resulting mixture was stirred for 4 days. The reaction mixture was partitioned between a 1N HCl solution (200 mL) and EtOAc (120 mL). The aqueous layer was extracted once again with EtOAc (150 mL). The combined organic fractions was washed with 1N HCl (100 mL) followed by water (100 mL), then dried over sodium sulfate, filtered and evaporated to dryness. The residue was purified by flash column chromatography on silica gel (100% hexanes and 20% EtOAc in hexanes as eluent) to afford Boc-D-Trp-O-cyclohexyl as a yellowish solid (15.4 g). Yield=81%; MS-ESI (m/z): 387 [M+1]⁺.

To a solution of Boc-D-Trp-O-cyclohexyl (13.8 g, 35.8 mmol) in CH₂Cl₂ cooled to 5-10° C. was bubbled HCl gas for 30 min. The resulting solid suspension was collected by suction filtration, washed with CH₂Cl₂ (2×100 mL), then dried in a vacuum oven at 42° C. for 6 h. Thus, H-D-Trp-O-cyclohexyl hydrochloride salt was obtained (6.4 g) as a solid. ¹H NMR (DMSO-D₆, 400 MHz) δ ppm: 11.12 (br. P s, 1H), 8.68 (br.s, 3H), 7.55 (d, J=7.8 Hz, 1H), 7.37 (d, J=8.0 Hz, 1H), 7.24 (s, 1H), 7.07 (t, J=7.7 Hz, 1H), 7.02 (t, J=7.4 Hz, 1H), 4.64 (apparent br. t, 1H), 4.13 (apparent br. t, 1H), 3.30-3.35 (m, 1H), 3.22-3.28 (m, 1H), 1.44-1.50 (m, 1H), 1.35-1.65 (m, 4H), 1.10-1.30 (m, 5H); MS-ESI (m/z): 287 [M+1]+(free base).

### B. Preparation of Cbz-D-Glu(D-Trp-O-cyclohexyl)-O-Bzl

To a solution of Cbz-D-Glu-O-Bzl (2.8 g, 7.6 mmol), EDC.HCl (2.2 g, 11.4 mmol), HOBt hydrate (1.5 g, 11.4 mmol) and DIPEA (3.3 mL, 19.0 mmol) was added H-D-Trp-O-cyclohexyl hydrochloride salt (3.2 g, 9.9 mmol) at RT. The mixture was stirred under a blanket of nitrogen for overnight. The mixture was evaporated to dryness in vacuo and the residue was partitioned between a 5% sodium carbonate solution (150 mL) and EtOAc (150 mL). The aqueous layer was extracted once again with EtOAc (150 mL). The combined organic fractions was successively washed with a 5% sodium carbonate solution (100 mL), a 1N HCl solution (2×100 mL) and water (100 mL), then dried over sodium sulfate, filtered and concentrated in vacuo. Purification of the residue by column chromatography on silica gel (20 to 40% EtOAc in hexanes then 10% MeOH in EtOAc as eluent) afforded Cbz-D-Glu(D-Trp-O-cyclohexyl)-O-Bzl in quantitative yield. ¹H NMR (DMSO-D₆, 400 MHz) δ ppm: 10.88 (br. s, 1H), 8.35 (d, J=7.0 Hz, 1H), 7.82 (d, J=7.0 Hz, 1H), 7.49 (d, J=7.0 Hz, 1H), 7.25-7.40 (m, 11H), 7.18 (s, 1H), 7.08 (t, J=7.4 Hz, 1H), 6.98 (1, J=7.4 Hz, 1H), 5.13 (s, 2H), 5.02-5.10 (m, 2H), 4.56-4.62 (m, 1H), 4.48 (apparent q, J=7.2 Hz, 1H), 4.05-4.12 (m, 1H), 3.04-3.12 (m, 1H), 2.98-3.06 (m, 1H), 2.15-2.25 (m, 2H), 1.90-2.00 (m, 1H), 0.90-1.80 (m, 11H); MS-ESI (m/z): 640 [M+1]⁺.

### C. Preparation of H-D-Glu(D-Trp-O-cyclohexyl)-OH (Apo850)

A solution of Cbz-D-Glu(D-Trp-O-cyclohexyl)-O-Bzl (2.89 g, 4.52 mmol) and 10% wet Pd/C (387 mg) in EtOH (180 mL) was subjected t hydrogenolysis under a hydrogen pressure of 15 psi for 2.75 h. The mixture was filtered over a pad of Celite™ and the filtrate was evaporated to dryness. Purification of the residue by column chromatography on silica gel (5 to 20% MeOH in CH₂Cl₂) afforded H-D-Glu(D-Trp-O-cyclohexyl)-OH (Apo850, 1.40 g) as a white solid. Yield=75%; ¹H NMR (DMSO-D₆, 400 MHz) δ ppm: 11.01 (br. s, 1H), 8.78 (d, J=7.2 Hz, 1H), 7.50-8.20 (br above baseline hump), 7.49 (d, J=7.8 Hz, 1H), 7.34 (d, J=8.0 Hz, 1H), 7.18 (s, 1H), 7.06 (t, J=7.2 Hz, 1H), 6.98 (t, J=7.5 Hz, 1H), 4.60 (br apparent t, 1H), 4.48 (apparent q, J=6.9 Hz, 1H), 3.20-3.60 (br above baseline hump), 3.30 (t, J=6.3 Hz, 2H), 3.10-3.18 (m, 1H), 2.98-3.06 (m, 1H), 2.24-2.32 (m, 2H), 1.83-1.87 (m, 2H), 1.50-1.70 (m, 4H), 1.15-1.45 (m, 6H); MS-ESI (m/z): 416 [M+1]⁺.

### Example 30

### Preparation of H-D-Glu(D-Trp-OCH₂O—CO—C(CH₃)₃)—OH, Apo839

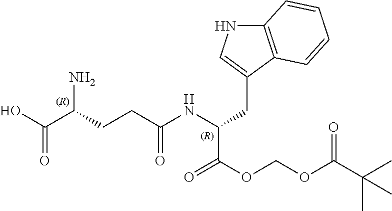

Proceeding in a similar manner as described in Example 12, by replacing chloromethyl benzoate with chloromethyl pivalate, H-D-Glu(D-Trp-OCH₂O—CO—C(CH₃)₃)—OH (Apo839) was prepared. ¹H NMR (DMSO-D₆, 400 MHz) δ ppm: 11.09 (br. 1H), 8.83 (d, J=7.1 Hz, 1H), 7.50-8.20 (br above baseline hump), 7.47 (d, J=7.8 Hz, 1H), 7.34 (d, J=8.0 Hz, 1H), 7.21 (d, J=2.1 Hz, 1H), 7.06 (t, J=7.1 Hz, 1H), 6.98 (t, J=7.4 Hz, 1H), 5.68-5.76 (m, 2H), 4.42-4.48 (m, 1H), 3.40-3.70 (br. above baseline hump), 3.29 (t, J=6.5 Hz, 1H). 3.12-3.15 (m, 1H), 3.00-3.08 (m, 1H), 2.26-2.30 (m, 2H), 1.46-1.52 (m, 2H), 1.12 (s, 9H); MS-ESI (m/z): 448 [M+1]⁺.

### Example 31

### Preparation of H-D-Glu(D-Trp-OH)—OMe (Apo841)

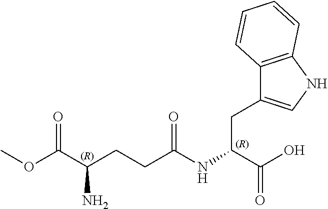

A mixture of Boc-D-Glu(D-Trp-OH)—OBzl (Example 6A, 2.10 g, 4.00 mmol) and sodium methoxide (0.55 g, 10.0 mmol) in methanol (60 mL) was stirred at RT for 25 min. The reaction mixture was quenched with acetic acid (0.6 mL, 10.5 mmol), then evaporated to dryness in vacuo to afford crude Boc-D-Glu(D-Trp-OH)—OMe as an oil.

The residual oil was taken up in CH₂Cl₂ (180 mL), then washed with a mixture of de-ionized water (50 mL) and acetic acid (0.3 mL). The organic solution was dried over Na₂SO₄, filtered, and the volume of the filtrate was reduced to about 80 mL via rotary evaporation. The organic layer was cooled in an ice-water bath, as HCl gas was bubbled in slowly. The progress of the reaction was monitored by HPLC. The upper liquid was decanted, and the sticky solid was triturated with more CH₂Cl₂. The sticky solid was then dissolved in water (30 mL) and the pH of the solution was adjusted to about 5.5 by with a 6N NaOH solution (0.5 mL, 3 mmol). Acetonitrile (100 mL) was then added, and the mixture was evaporated to dryness in vacuo to give an oil. Upon trituration with ethyl acetate a solid formed. The solid was collected via suction filtration, thoroughly washed with water and dried to afford H-D-Glu(D-Trp-OH)—OMe (Apo822, 0.49 g). Yield=35%; HPLC (AUC) purity at 280 nm=98.1%; ¹H NMR (DMSO-D₆) δ ppm: 10.82 (s, 1H), 8.07 (d, J=7.8 Hz, 1H), 7.52 (d, J=7.8 Hz, 1H), 7.32 (d, J=8.0 Hz, 1H), 7.12 (s, 1H), 7.05 (t. J=7.4 Hz, 1H), 6.97 (t, J=7.4 Hz, 1H), 4.39-4.45 (m, 1H), 3.60 (s, 3H), 3.28-3.31 (m, 1H), 3.14-3.19 (m, 1H), 2.95-3.01 (m, 1H), 2.15-2.19 (m, 2H), 1.73-1.82 (m, 1H), 1.54-1.63 (m, 1H); MS-ESI (m/z): 348 [M+1]⁺.

### Example 32

### Preparation of a mixture of D-Glu(D-Trp-OCH₂CF₃)—OH and D-Glu(L-Trp-OCH₂CF₃)—OH, Apo860

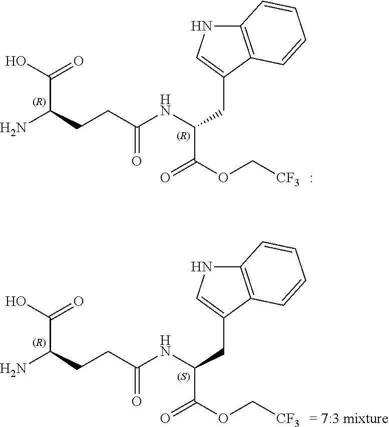

### A. Preparation of Cbz-D-Glu(D-Trp-OH)—OBn

To an ice-water cooled solution of Cbz-D-Glu-OBzl (20.0 g, 53.9 mmol) in DMF (100 mL) was added N-hydroxysuccinimide (6.82 g, 59.2 mmol), followed by EDCl.HCl (11.4 g, 59.2 mmol), and the mixture was stirred at RT for overnight. The mixture was then cooled in an ice-water bath, and H-D-Trp-OH (12.1 g, 59.2 mmol) was added, followed by DIPEA (10 mL). The mixture was stirred for overnight. The reaction mixture was quenched with a 0.5N HCl, solution and then extracted with EtOAc. The EtOAc layer was washed with a 10% citric acid solution and brine, dried over anhydrous Na₂SO₄, filtered and concentrated to dryness. The residue was triturated with Et₂O, and the solid was collected via filtration to afford Cbz-D-Glu(D-Trp-OH)—OBn (26.1 g). Yield=87%; MS-ESI (m/z): 558 [M+1]⁺.

### B. Preparation of a mixture of D-Glu(D-Trp-OCH₂CF₃)—OH and D-Glu(L-Trp-OCH₂CF₃)—OH, Apo860

To an ice-water cooled solution of Cbz-D-Glu(D-Trp-OH)—OBzl (3.5 g, 6.3 mmol) in DMF (50 mL) was added N-hydroxysuccinimide (0.8 g, 6.9 mmol), followed by EDCl.HCl (1.32 g, 6.9 mmol), and the mixture was stirred at RT for overnight. The mixture was then cooled in an ice-water bath and DIPEA (1.23 mL, 6.9 mmol) was added, followed by 2.2.2-trifluoroethanol (1.8 mL, 25.1 mmol). The mixture was stirred at RT for 5 h. The reaction mixture was then partitioned between water and EtOAc. The EtOAc layer was collected and washed with brine, dried over anhydrous Na₂SO₄, filtered and concentrated to dryness. The residue was triturated with hexanes. The hexanes layer was discarded. The crude residue was mixed with 0.95 g of wet 10% Pd—C in EtOH (100 mL), and was hydrogenated under a blanket of hydrogen at 45 psi hydrogen pressure in a Parr apparatus for 3 h. The mixture was filtered, and the filtrate was concentrated to dryness in vacuo. The residue was triturated with a mixture of acetone, EtOAc and hexanes. The solid was collected via filtration, and was further purified by flash column chromatography on silica gel using a solvent mixture of IPA/H₂O (85/15 ratio, v/v) as eluent to afford D-Glu(D-Trp-OCH₂CF₃)—OH (1.16 g). Yield=44%; ¹H NMR (DMSO-D₆+D₂O, 400 MHz): δ (ppm) 7.47 (d, J=8.1 Hz, 1H), 7.35 (d, J=8.1 Hz, 1H), 7.17 (s, 1H), 7.04-7.12 (m, 1H), 6.96-7.04 (m, 1H), 4.57-4.75 (m, 2H), 4.45-4.57 (m, 1H), 3.29 (t, J=6.1 Hz, 0.7H), 3.23 (t, J=6.1 Hz, 0.3H), 318 (d, J=6.1 Hz, 0.3H), 3.15 (d, J=6.1 Hz, 0.7H), 3.01-3.12 (m, 1H), 2.14-2.39 (m, 2H), 1.68-1.97 (m, 2H). MS-ESI (m/z): 416 [M+1]⁺.

The ¹H NMR data indicates the presence of about 30% of D-Glu(L-Trp-OCH₂CF₃)—OH

### Example 33

### Preparation of H-D-Glu(D-Trp-OCH₂CF₃)—OCH₂CF₃ hydrochloride salt, Apo868.HCl

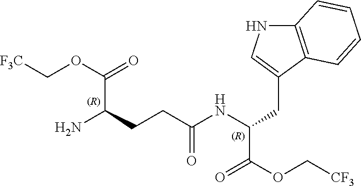

### A. Preparation of Boc-D-Glu (D-Trp-OCH₂CF₃)—OCH₂CF₃

To an ice-water cooled solution of Boc-D-Glu(D-Trp-OH)—OH (6.0 g, 13.8 mmol) in DMF (50 mL) was successively added N-hydroxysuccinimide (3.5 g, 30.5 mmol), EDCl.HCl (5.8 g, 30.5 mmol), 2.2.2-trifluoroethanol (6 mL, 83.1 mmol) and DIPEA (5.3 mL, 30.5 mmol). The resulting solution was then stirred at RT for overnight. The reaction mixture was quenched with water, and then extracted with EtOAc. The EtOAc layer was washed with a 10% citric acid solution and brine, and then dried over anhydrous Na₂SO₄, filtered and concentrated to dryness under reduced pressure. The residue was purified by flash column chromatography on silica gel using a mixture of EtOAc and Hexanes (1/1 ratio, v/v) as eluent to afford Boc-D-Glu (D-Trp-OCH₂CF₃)—OCH₂CF₃ (6.1 g). Yield=74%); MS-ESI (m/z): 598 [M+1]⁺.

### B. Preparation of H-D-Glu (D-Trp-OCH₂CF₃)—OCH₂CF₃ hydrochloride salt, Apo868.HCl

To an ice-water cooled solution of Boc-D-Glu (D-Trp-OCH₂CF₃)—OCH₂CF₃ (6.0 g, 10.0 mmol) in EtOAc was bubbled HCl gas for 35 min. The reaction mixture was concentrated to dryness to give a crude product (5.4 g). A portion of the crude material (1.0 g) was purified by flash column chromatography on silica gel using a mixture of EtOAc and MeCN (gradient from 10/0 to 1/1 ratio, v/v) as eluent to afford H-D-Glu (D-Trp-OCH₂CF₃)—OCH₂CF₃ hydrochloride salt (625 mg). ¹H NMR (DMSO-D₆, 400 MHz): δ (ppm) 10.95 (s, 1H), 8.66-8.72 (m, 4H), 7.48 (d, J=7.8 Hz, 1H), 7.35 (d, J=8.0 Hz, 1H), 7.19 (s, 11-1), 7.06-7.09 (m, 1H), 6.98-7.01 (m, 1H), 4.86-4.92 (m, 2H), 4.69-4.77 (m, 2H), 4.52-4.56 (m, 1H), 4.18-4.20 (m, 1H), 3.07-3.20 (m, 2H), 2.30-2.41 (m, 2H), 1.97-2.01 (m, 2H); MS-ESI (m/z): 498 [M+1]⁺.

### Example 34

### Preparation of (R)-2,3-dihydro-1H-inden-5-yl 5-((S)-3-(1H-indol-3-yl)-1-(isopentyloxy)-1-oxopropan-2-ylamino)-2-amino-5-oxopentanoate hydrochloride or H-D-Glu(L-Trp-O-isoamyl)-O-2,3-dihydro-1H-inden-5-yl.HCl or (Apo928.HCl)

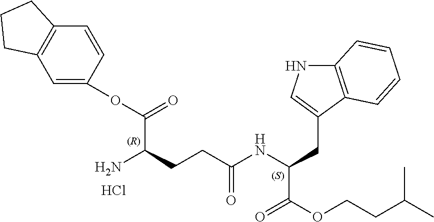

### A. Preparation of Boc-L-Trp-O-isoamyl

Boc-L-Trp-O-isoamyl was prepared from the reaction of Boc-L-Trp-OH (10.0 g, 32.8 mmol), 3-methyl-1-butanol (7.1 mL, 65.7 mmol) with HOBt (5.3 g, 39.4 mmol), DIPEA (7.4 mL, 42.7 mmol) and EDGE (8.2 g, 42.7 mmol) in DMF (100 mL). The resulting mixture was stirred at room temperature for overnight. The reaction mixture was poured into a beaker of cold water (100 mL) with stirring, and the resulting suspension was stirred at 5° C. (ice bath) for 20 min. Suction filtration afforded Boc-L-Trp-O-isoamyl as a white solid, which was air-dried for overnight (10.8 g). Yield=88%; ¹H NMR (DMSO-d₆, 400 MHz) δ ppm: 10.86 (br. s., 1H), 7.48 (d, J=8.1 Hz, 1H), 7.34 (d, J=8.1 Hz, 1H), 7.22 (d, J=7.1 Hz, 1H), 7.16 (s, 1H), 7.07 (t, J=7.1 Hz, 1H), 6.99 (t, J=7.6 Hz, 1H), 4.12-4.24 (m, 1H), 3.93-4.09 (m, 2H), 3.05-3.15 (t, 1H), 2.95-3.05 (m, 1H), 1.48-1.59 (m, 1H), 1.31-1.41 (m, 11H), 0.82 (t, J=6.6 Hz, 6H); MS-ESI (m/z) 375 [M+1]⁺.

### B. Preparation of H-L-Trp-O-isoamyl hydrochloride

HCl gas was bubbled into a suspension of Boc-L-Trp-O-isoamyl (10.52 g, 28.1 mmol) in 150 ml EtOAc for 1.5 h. The suspension was stirred at 5° C. (ice-bath) for 20 min. The solid product was collected by suction filtration, and washed with EtOAc (3×15 mL) to afford H-L-Trp-O-isoamyl hydrochloride as white solid (7.83 g). Yield: 90%; ¹H NMR (DMSO-d₅, 400 MHz) δ ppm: 11.13 (br. s., 1H), 8.66 (br. s., 2H), 7.52 (d, J=8.1 Hz, 1H), 7.38 (d, J=8.1 Hz, 1H), 7.25 (s, 1H), 7.09 (t, J=7.6 Hz, 1H), 7.01 (t, J=7.6 Hz, 1H), 4.19 (t, J=6.6 Hz, 1H), 3.94-4.08 (m, 2H), 3.33 (d, J=5.1 Hz, 1H), 3.20-3.29 (m, 1H), 1.36-1.48 (m, 1H), 1.23-1.33 (m, 2H), 0.78 (d, J=5.1 Hz, 6H); MS-ESI (m/z) 275 [M+1]+(free base).

### C. Preparation of Boc-D-Glu(L-Trp-O-isoamyl)-O-bzl

Boc-D-Glu(L-Trp-O-isoamyl)-O-bzl was prepared from the reaction of H-L-Trp-O-isoamyl hydrochloride (7.65 g, 24.6 mmol), Boc-D-Glu-O-bzl (8.3 g, 24.6 mmol), EDCl (5.67 g, 29.5 mmoL), HOBt (3.5 g, 25.8 mmol) and DIPEA (8.6 mL, 49.2 mmol) in DMF (100 mL). The resulting mixture was stirred at room temperature for overnight. The reaction mixture was poured into a beaker of cold water (250 mL) with stirring. The mixture was extracted with ethyl acetate (100 mL×3). The combined organic layers was successively washed with a 10% citric acid solution (30 mL), a saturated NaHCO₃ (50 mL) and brine (50 mL), and was then dried over MgSO₄. After solvent was removed in vacuo, Boc-D-Glu(L-Trp-O-isoamyl)-O-bzl was obtained as light yellowish oil (13.5 g). Yield=93%; ¹H NMR (DMSO-d₆, 400 MHz) δ ppm: 10.87 (br. s., 1H), 8.30 (d, J=7.1 Hz, 1H), 7.48 (d, J=8.1 Hz, 1H), 7.27-7.40 (m, 7H), 7.15 (br. s., 1H), 7.07 (t, J=7.6 Hz, 1H), 6.91-7.03 (m, 1H), 5.04-5.19 (m, 2H), 4.48 (d, J=6.1 Hz, 1H), 3.97 (t, J=6.1 Hz, 3H), 3.12 (dd, J=14.1, 6.1 Hz, 1H), 3.02 (dd, J=14.1, 8.1 Hz, 1H), 2.14-2.29 (m, 2H), 1.93 (d, J=8.1 Hz, 1H), 1.67-1.83 (m, 1H), 1.41-1.55 (m, 2H), 1.28-1.38 (m, 10H), 0.80 (t, J=6.1 Hz, 6H); MS-ESI (m/z) 594 [M+1]⁺.

### D Preparation of Boc-D-Glu(L-Trp-O-isoamyl)-OH

A mixture of Boc-D-Glu(L-Trp-O-isoamyl)-O-benzyl (12.35 g, 20.8 mmol) and 1.5 g of 10% Pd on activated carbon (wet) in ethanol (250 ml) was shaken in a Parr apparatus under a hydrogen atmosphere at a pressure of 45 psi at room temperature for 2 h. The Pd catalyst was filtered through Celite™ and the filtrate was evaporated under reduced pressure to give a pink oil, which was dried under vacuum to afford Boc-D-Glu(L-Trp-O-isoamyl)-OH (9.1 g) as a pink foamy solid. Yield=87%; ¹H NMR (DMSO-d₆, 400 MHz) δ ppm: 10.87 (s, 1H), 8.30 (d, J=7.1 Hz, 1H), 7.48 (d, J=7.1 Hz, 1H), 7.34 (d, J=8.1 Hz, 1H), 7.15 (s, 1H), 7.03-7.12 (m, 2H), 6.93-7.03 (m, 1H), 4.41-4.54 (m, 1H), 3.98 (t, J=6.6 Hz, 2H), 3.82-3.92 (m, 1H), 3.39-3.50 (m, 2H), 3.07-3.18 (m, 1H), 2.97-3.07 (m, 1H), 2.18 (t, J=7.6 Hz, 2H), 1.90 (d, J=8.1 Hz, 1H), 1.70 (dd, J=13.6, 7.6 Hz, 1H), 1.47 (dq, J=13.3, 6.7 Hz, 1H), 1.26-1.41 (m, 9H), 1.07 (t, J=6.6 Hz, 1H), 0.75-0.84 (m, 6H); MS-ESI (m/z) 504 [M+1]⁺.

### E. Preparation of Boc-D-Glu(L-Trp-O-isoamyl)-O-2,3-dihydro-1H-inden-5-yl

5-indanol (0.43 g, 3.23 mmol) was added to a solution of Boc-D-Glu(L-Trp-O-isoamyl)-OH (1.25 g, 2.48 mmol) in DMF (35 mL) followed by EDCl (0.62 g, 3.23 mmol), HOBt (0.40 g, 2.98 mmol) and DIPEA (0.62 mL, 3.48 mmol). The resulting mixture was stirred at room temperature for overnight. The reaction mixture was poured into a beaker of cold water (100 mL) with stirring. The mixture was extracted with ethyl acetate (50 mL×3). The combined organic layers was successively washed with a 10% citric acid solution (20 mL), a saturated NaHCO₃ solution (25 mL) and brine (40 mL). The organic fraction was dried over MgSO₄. After solvent was removed in vacuo, the crude product was obtained as light yellowish oil. The oil was further purification by flash chromatography on silica gel using a solvent mixture of EtOAc and Hexanes (1/1 ratio, v/v) as eluent to give Boc-D-Glu(L-Trp-O-isoamyl)-O-2,3-dihydro-1H-inden-5-yl as a light yellowish foamy solid (1.36 g). Yield: 91%; ¹H NMR (DMSO-d₆, 400 MHz) δ ppm: 10.87 (s, 1H), 8.37 (d, J=7.1 Hz, 1H), 7.48 (d, J=8.1 Hz, 2H), 7.34 (d, J=8.1 Hz, 1H), 7.21-7.27 (m, J=8.1 Hz, 1H), 7.15 (s, 1H), 7.07 (t, J=7.1 Hz, 1H), 6.98 (1, J=7.1 Hz, 1H), 6.92 (s, 1H), 6.76-6.84 (m, J=8.1 Hz, 1H), 4.43-4.55 (m, 1H), 4.07-4.19 (m, 1H), 3.98 (t, J=6.6 Hz, 2H), 3.13 (dd, J=6.1, 14.2 Hz, 1H), 3.03 (dd, J=8.1, 14.2 Hz, 1H), 2.80-2.90 (m, 4H), 2.24-2.34 (m, 2H), 1.98-2.10 (m, 3H), 1.82-1.95 (m, 1H), 1.46 (dd, J=6.6, 13.6 Hz, 1H), 1.41 (s, 8H), 1.27-1.36 (m, 2H), 0.80 (t, J=6.6 Hz, 6H); MS-ESI (m/z) 620 [M+1]⁺.

### F. Preparation of H-D-Glu(L-Trp-O-isoamyl)-O-2,3-dihydro-1H-inden-5-yl hydrochloride (Apo928.HCl)

HCl gas was bubbled into a solution of Boc-D-Glu(L-Trp-O-isoamyl)-O-2,3-dihydro-1H-inden-5-yl (0.72 g, 1.16 mmoL) in 35 mL dichloromethane for 3.5 h. The reaction mixture was evaporated to dryness and the crude product was further purified by flash chromatography on silica gel using a solvent mixture of isopropyl alcohol and dichloromethane (1/1 ratio, v/v) as eluant to give the sticky foamy solid. The foamy solid was then dissolved in a 2M HCl Et₂O solution, and stirred at room temperature for 15 min. After evaporation of volatiles in vacuo of H-D-Glu(L-Trp-O-isoamyl)-O-2,3-dihydro-1H-inden-5-yl hydrochloride (Apo928.HCl) was obtained as a brown foamy solid (0.34 g). Yield: 52%; ¹H NMR (DMSO-d₅, 400 MHz,) δ ppm: 10.93 (s, 1H), 8.79 (br. s., 2H), 8.62 (d, J=7.1 Hz, 1H), 7.48 (d, J=8.1 Hz, 1H), 7.34 (d, J=8.1 Hz, 1H), 7.28 (d, J=8.1 Hz, 1H), 7.17 (s, 1H), 7.02-7.10 (m, 2H), 6.92-7.01 (m, 2H), 4.50 (q, J=7.1 Hz, 1H), 4.27 (br. s., 1H); 3.97 (t, J=6.6 Hz, 2H), 3.10-3.18 (m, 1H), 3.01-3.10 (m, 1H), 2.87 (q, J=7.1 Hz, 4H), 2.45-2.50 (m, 1H), 2.33-2.45 (m, 1H), 2.10-2.20 (m, 2H), 1.99-2.10 (m, 2H), 1.46 (dt, J=6.6, 13.1 Hz, 1H), 1.26-1.36 (m, 2H), 0.80 (t, J=6.6 Hz, 6H); MS-ESI (m/z) 520[M+1]⁺ (free base).

### Example 35

### A. Biotransformation Studies of a Compound of Formula I in Human Hepatocytes

### General Procedure:

LiverPool® cryopreserved human hepatocytes (pooled from 10 male donors) was obtained from Celsis In Vitro Technologies. The hepatocytes were stored in liquid nitrogen until used. Just before the assay, the hepatocytes were quickly thawed at 37° C. and centrifuged at 100×g for 10 min. The media was removed and cells were re-suspended in PBS at a density of 4×10⁶ cells/mL. The compound of formula I (100 μM) was incubated with 0.1×10⁶ hepatocytes in 50 μL volume. After 10, 20, 60, 120 and 240 min of incubation, the reaction was quenched by adding an equal volume of 5% (w/v) TCA. The “time 0” sample was generated by adding TCA before the test compound. After brief vortexing and 10-min incubation on ice, samples were centrifuged (16,000×g, 10 min) and the supernatants were analyzed by HPLC with UV detection.

HPLC analysis of pro-drugs in SGF, SIF, plasma and hepatocytes samples:

HPLC analysis was done using an Agilent 1100 series HPLC system consisting of a programmable multi-channel pump, auto-injector, vacuum degasser and HP detector controlled by Agilent HPLC218 Chem Station Rev.A.09.03 software for data acquisition and analysis. A gradient method was used for the determination of all pro-drugs and their hydrolysis products including Apo805 on an Agilent Eclipse XDB, C18 column (part #963967-902, 150×4.6 mm, 3.5 μm) with the following chromatographic conditions:
Temperature: Ambient
Mobile phase: A=Aqueous phase: 10 mM Tris-HCl, 2 mM EDTA, pH 7.4 B=Organic phase: Acetonitrile
Gradient method: Time: 0 min 5% B, 25 min 50% B, 35 min 80% B, 45 min 5% B, 50 min 5% B.
Mobile phase flow rate: 1.0 mL/min
Injection volume: 50 μL
Data acquisition time: 30 min
Detection wavelength: 280 nm; 4 nm bandwidth, ref. 360 nm,
   4 nm bandwidth
      The chromatograms at λ=280 nm were analyzed. Peak area (mAU*s) was used for quantitation of pro-drugs, intermediates and thymodepressin (Apo805).

### B. Stability in Human Blood

Blood was collected from healthy volunteers, both male and female, in Becton Dickinson ACD Vacutainer™ containing ACD solution A (22.0 g/L trisodium citrate, 8.0 g/L citric acid, 24.5 g/L dextrose). Blood from the vacutainers was pooled in a 50 mL Falcon™ tube, kept on ice, and used in the assay within 2 hours of collection. To determine rate of hydrolysis, each prodrug (100 μM) was incubated in pooled human blood at 37° C. Immediately after test compound addition and after 0.5, 1, 2, 4, 6 and 24 hour incubation, blood aliquots (500 μL) were removed and centrifuged at 1500×g, for 10 min at 4° C. An aliquot of plasma (150 μL) was transferred to an eppendorf tube and the plasma proteins were precipitated by adding an equal volume of 5% TCA (w/v). After 10-min incubation on ice, samples were centrifuged (16,000×g, 10 min) and the supernatants were analyzed by HPLC.

The biotransformation data of a compound of formula I in human blood and human hepatocytes are shown in Tables 1 to 3 below:

TABLE 1
In vitro bioconversion of H-D-Glu(Trp-O-T)-OH to
Apo805 in human hepatocytes and blood.

| | | | Half-life for | |
|---|---|---|---|---|
| | | | bioconversion | |
| | | | to Apo805*(h) | |
| Compound | | | Human | Human |
| ID | Stereochemistry | T | hepatocytes | blood |
| Apo835 | D, D | ethyl | >2.0 (11% in 2 h) | >24 |
| Apo839 | D, D | CH₂O—CO—t-Bu | 0.4 | 1.3 |
| Apo843 | D, D | CH(Me)—O—CO—O-cyclohexyl | 0.2 | 4.3 |
| Apo849 | D, D | Mofetil | 2.7 | 3.1 |
| Apo851 | D, D | indanyl | 0.3 | >23 |
| Apo852 | D, D | 2-methoxyphenyl | 0.9 | 14 |
| Apo860 | D, D | OCH₂CF₃ | 2.9 | 5.7 |
| Apo877 | D, D | CH₂CH₂CF₃ | >6.0 (19% in 6 h) | >24 |
| Apo888 | D, D | CH(Me)—O—CO—O—CH₂CH₃ | 1.1 | 1.5 |
| Apo891 | D, D | CH(Me)—O—CO—O—i-Pr | 0.8 | 1.8 |
| Apo893 | D, D | CH₂—CO—N(CH₃)₂ | >6.0 (15% in 6 h) | >24 |
| Apo895 | D, D | CH₂—O—CO—C(Me)₂—CH₂CH₂CH₃ | 0.7 | 2.1 |

Selected compounds of formula I with the formula H-D-Glu(D-Trp-OR²)—OR¹ show better t_{1/2} in its biotransformation in human hepatocytes and human blood to thymodepressin (Apo805, H-D-Glu(D-Trp-OH)—OH than the monoethyl ester Apo835 H-D-Glu(D-Trp-OEt)-OH, white the peptide amide Apo893 is not readily converted to thymodepressin in human hepatocytes.

TABLE 2
In vitro bioconversion of H-Glu(Trp-OH)—O-G to
Apo805 in human hepatocytes and blood.

| | | | Half-life for bioconversion | |
|---|---|---|---|---|
| | | | to Apo805* (h) | |
| Compound | Stereo- | | Human | Human |
| ID | chemistry | G | hepatocytes | blood |
| Apo829 | D, D | Benzyl | >2.0 (21% in 2 h) | >24 |
| Apo836 | D, D | Et | >2.0 (12% in 2 h) | >24 |
| Apo841 | D, D | Me | >3.0 (25% in 3 h) | 15 |
| Apo846 | D, D | i-Pr | >3.0 (9% in 3 h) | >24 |
| Apo865 | D, D | CH₂CF₃ | 0.5 | 0.6 |
| Apo878 | D, D | CH₂CH₂CF₃ | >4.0 (41% in 4 h) | 8.9 |
| Apo886 | D, L | CH₂CH₂CF₃ | 4.6 | 5.9 |

*For pro-dugs for which bioconversion half-life was not measured, values in parentheses indicate percent conversion to Apo805 within indicated time.

When compared to the monoalkyl ester derivatives H-D-Glu(D-Trp-OH)—O—R³ such as Apo829, Apo836, Apo841 and Apo846, the fluoroalkyl derivatives H-D-Glu(D-Trp-OH)—O—(CH₂)ₙCF₃ show a faster rate of biotransformation to Apo805 in human hepatocytes.

TABLE 3
In vitro bioconversion of H-Glu(Trp-O-T)-O-G to
Apo805 in human hepatocytes and blood.

| | | | | Bioconversion to Apo805 | |
|---|---|---|---|---|---|
| | | | | after 4 h (%) | |
| Compound | | | | Human | Human |
| ID | Stereochemistry | G | T | hepatocytes | blood |
| Apo854 | D, D | Et | CH(Me)—O—CO—O-cyclohexyl | 23 | 4 |
| Apo900 | D, D | Et | 2-morpholinylethyl | 31 | 7 |
| Apo879 | D, D | CH₂CH₂CF₃ | CH₂CH₂CF₃ | 23 | 13 |

### Example 36

### Pharmacokinetic Studies of a Compound of Formula I in Rats

### General Procedure for Animal Dosing

Groups of five male Sprague-Dawley rats weighing 250 to 300 g were utilized per dosing group. One day prior to dosing, venous and arterial catheters (made of 20 cm long polyurethane coiled tubing, and filled with 100 units/mL heparinized saline) were implanted into the jugular vein and carotid artery of each rat. Rats were fasted overnight prior to oral dosing and fed approximately 2 hours post-dosing. All dosing and blood sampling was performed on fully conscious rats. Tested compounds were administered either by oral gavage as solutions in water, or by intravenous injection (Apo805K1 only) as solution in 0.9% sodium chloride, final pH 7.0, at doses equivalent to 5 mg/kg (per Apo805 content). Blood (0.3 mL) was sampled from each animal from the carotid artery for up to 30 hours post-dosing, each sampling followed by an equivalent naive-blood replacement. The blood sample was immediately centrifuged (4300×g for 5 minutes at 4° C.), and frozen at −80° C. until LC/MS/MS analysis.

### General Procedure for LC-MS/MS Analysis of Plasma Drug Concentration

Methanol (200 μL) was added to plasma samples (50 μL) to precipitate plasma proteins. After brief vortexing and centrifugation, the supernatant (200 uL) was removed and dried at 40° C. under a stream if nitrogen. The sample was reconstituted in water (300 μL) and 25 μL was injected for analysis.

A Sciex API 365 LC/MS/MS spectrophotometer equipped with Ionics EP10+ and HSID, was used, A chiral column (Supelco-Astec CHIROBIOTIC™ TAG), 100×2.1 mm, 5 μm was used at ambient temperature. The mobile phase consisted of 0.1% formic acid in water (A) and 0.1% formic acid in acetonitrile (B) in a ratio of 88:12 (A:B; v/v) and the flow rate was 0.6 mL/min. Positive ion electrospray ionization (ESI+) in MRM mode was used for analysis. Samples were analysed for the concentration of Apo805 (thymodepressin).

### PK Analysis

Non-compartmental analysis was performed using WinNonlin 52 software, on individual animal data. Bioavailability was calculated as a ratio of AUC_{INF}_{_{—}}_{D} after oral dosing of test compound to AUC_{INF}_{_{—}}_{D} after IV dosing of Apo805K1.

### Oral Bioavailability of Apo839 and Apo843 in Rats

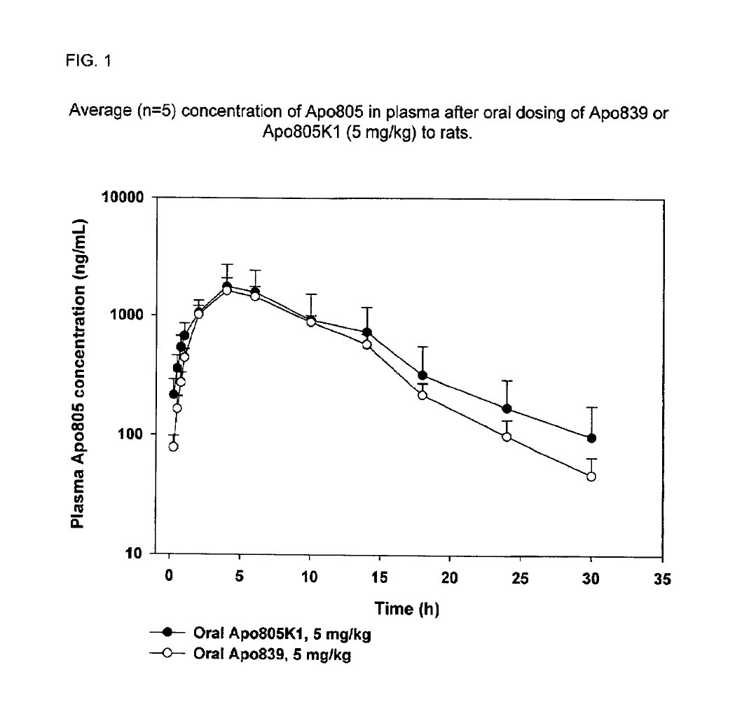

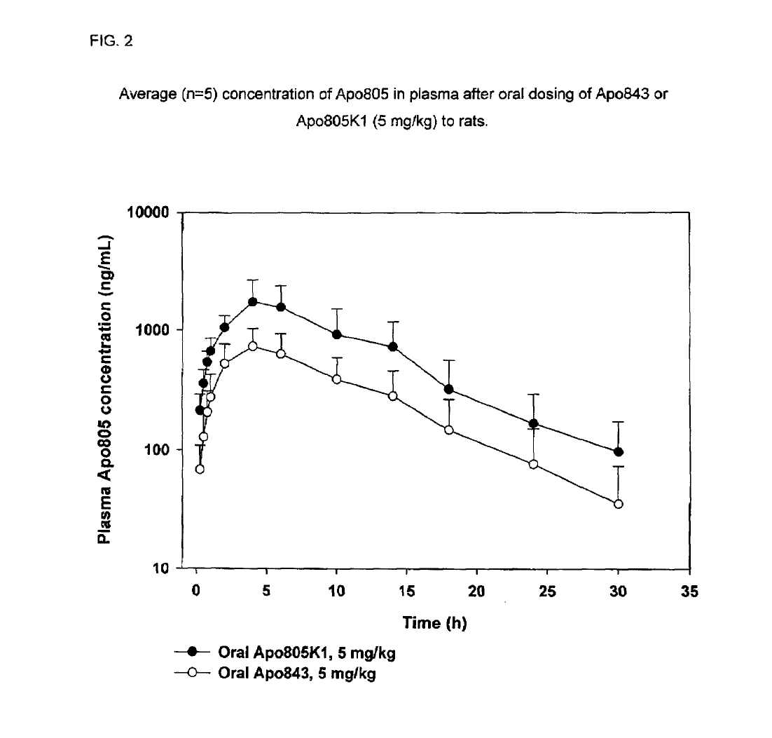

Absolute oral bioavailability of pro-drugs Apo839 and Apo843 was compared to that of Apo805K1 (potassium salt of thymodepressin) in male Sprague-Dawley rats. Adult animals, five per group, were dosed orally with 5 mg/kg Apo805K1, Apo839, or Apo843 and intravenously with 5 mg/kg Apo805K1. FIG. 1 shows the plasma concentration of Apo805 after oral dosing of Apo839 or Apo805K1. FIG. 2 shows the plasma concentration of Apo805 after oral dosing of Apo843 or Apo805K1. Apo839 and Apo843 show oral bioavailability and are transformed to thymodepressin (Apo805) in rats.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Furthermore, material appearing in the background section of the specification is not an admission that such material is prior art to the invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1-53. (canceled)

54. A compound of Formula I: 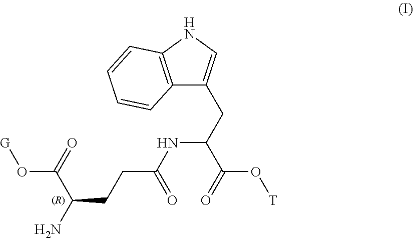
or a pharmaceutically acceptable salt thereof, wherein
G is selected from the group consisting of: H, 2-morpholinoethyl, (CH₂)ₙCF₃, C₁-C₈ alkyl, benzyl and A₅-A₁₀ aryl;
T is selected from the group consisting of: H, C₁-C₈ alkyl, 2-morpholinoethyl, (CH₂)ₙCF₃, CH₂CONR⁴R⁵, CH₂CH₂NR⁴R⁵, C₃-C₆ cycloalkyl, benzyl, A₅-A₁₀ aryl, 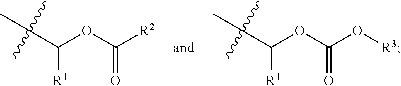
n is 1, 2, 3 or 4;
R¹ is H or C₁-C₈ alkyl;
R² is C₁-C₈ alkyl, C₃-C₆ cycloalkyl, or phenyl;
R³ is C₁-C₈ alkyl, C₃-C₆ cycloalkyl, or phenyl; and
R⁴ and R⁵ are either separate groups or together form a single group with the N to which they are bonded; when R⁴ and R⁵ are separate groups, R⁴ and R⁵ are independently selected from the group consisting of: C₁-C₆ alkyl; when R⁴ and R⁵ together with the N to which they are bonded form the single group, the single group is selected from the group consisting of: morpholinyl, N—(C₁-C₄ alkyl)-piperazinyl and piperidinyl;
provided that if T is H, then G is 2-morpholinoethyl, (CH₂)ₙCF₃, C₁-C₈ alkyl or benzyl; if T is CH₂CONR⁴R⁵, CH₂CH₂NR⁴R⁵, or C₃-C₆ cycloalkyl, then G is H; and if T is C₁-C₈ alkyl, then G is 2-morpholinoethyl, (CH₂)ₙCF₃, or A₅-A₁₀ aryl.

55. The compound of claim 54 wherein if G is H, then T is selected from the group consisting of: 2-morpholinoethyl, (CH₂)ₙCF₃, CH₂CONR⁴R⁵, CH₂CH₂NR⁴R⁵, C₃-C₆ cycloalkyl,

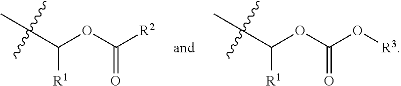

56. The compound of claim 54 wherein if G is H, then T is selected from the group consisting of: 2-morpholinoethyl, (CH₂)ₙCF₃, CH₂CH₂NR⁴R⁵, C₃-C₆ cycloalkyl,

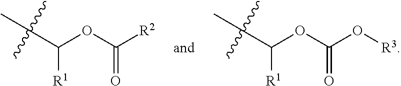

57. The compound of claim 54 wherein if G is H, then T is selected from the group consisting of: 2-morpholinoethyl, (CH₂)ₙCF₃, CH₂CH₂NR⁴R⁵,

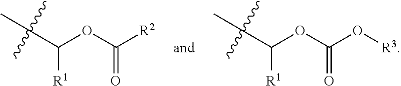

58. The compound of claim 54 wherein a chiral carbon of a tryptophan moiety is in the D-configuration.

59. The compound of claim 54 wherein a chiral carbon of a tryptophan moiety is in the L-configuration.

60. The compound of claim 54 wherein a chiral carbon of a tryptophan moiety is in the D-configuration or L-configuration and wherein G is H and T is A₅ to A₁₀ aryl.

61. The compound of claim 58 wherein T is

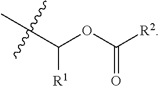

62. The compound of claim 58 wherein T is

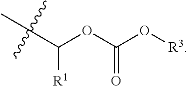

63. The compound of claim 58 wherein T is (CH₂)ₙCF₃.

64. The compound of claim 58 wherein T is 2-morpholinoethyl.

65. The compound of claim 58 wherein G is 2-morpholinoethyl, (CH₂)ₙCF₃, or C₁-C₈ alkyl; and T is 2-morpholinoethyl, (CH₂)ₙCF₃, A₅ to A₁₀ aryl,

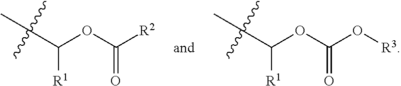

66. The compound of claim 58 wherein T is C₁-C₈ alkyl.

67. The compound of claim 66 wherein G is A₅ to A₁₀ aryl.

68. The compound of claim 67 wherein T is isoamyl, G is indanyl.

69. The compound of claim 58 wherein T is H.

70. The compound of claim 58 wherein G is H.

71. The compound of claim 58 wherein T is H and G is ethyl.

72. The compound of claim 58 wherein T is H and G is benzyl.

73. The compound of claim 58 wherein T is H and G is methyl.

74. The compound of claim 58 wherein T is H and G is isoamyl.

75. The compound of claim 58 wherein T is H and G is isopropyl.

76. The compound of claim 58 wherein T is H, G is (CH₂)ₙCF₃ and n is 1.

77. The compound of claim 58 wherein T is H, G is (CH₂)ₙCF₃ and n is 2.

78. The compound of claim 58 wherein T is H and G is 2-morpholinoethyl.

79. The compound of claim 58 wherein T is 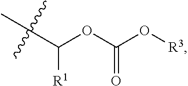
R¹ is methyl, R³ is cyclohexyl and G is H.

80. The compound of claim 58 wherein T is 2-morpholinoethyl and G is H.

81. The compound of claim 54 wherein a chiral carbon of a tryptophan moiety is in the D-configuration and wherein T is cyclohexyl and G is H.

82. The compound of claim 58 wherein T is (CH₂)ₙCF₃, n is 2 and G is H.

83. The compound of claim 58 wherein T is 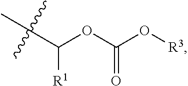
R¹ is methyl, R³ is ethyl and G is H.

84. The compound of claim 58 wherein T is 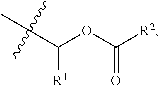
R¹ is H, R² is pent-2-yl and G is H.

85. The compound of claim 58 wherein T is 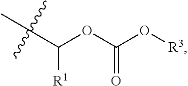
R¹ is methyl, R³ is isopropyl and G is H.

86. The compound of claim 54 wherein a chiral carbon of a tryptophan moiety is in the D-configuration and wherein T is CH₂CONR⁴R⁵, R⁴ is CH₃, R⁵ is CH₃ and G is H.

87. The compound of claim 54 wherein a chiral carbon of a tryptophan moiety is in the L-configuration and wherein T is CH₂CONR⁴R⁵, R⁴ is CH₃, R⁵ is CH₃ and G is H.

88. The compound of claim 58 wherein T is 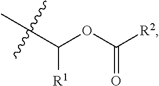
R¹ is H, R² is C(CH₃)₂—CH₂CH₂CH₃ and G is H.

89. The compound of claim 58 wherein T is (CH₂)ₙCF₃, n is 1 and G is H.

90. The compound of claim 59 wherein T is (CH₂)ₙCF₃, n is 1 and G is H.

91. The compound of claim 54 wherein a chiral carbon of a tryptophan moiety is in the D-configuration and wherein T is indanyl and G is H.

92. The compound of claim 54 wherein a chiral carbon of a tryptophan moiety is in the D-configuration and wherein T is 2-methoxyphenyl and G is H.

93. The compound of claim 58 wherein T is 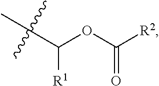
R¹ is H, R² is t-butyl and G is H.

94. The compound of claim 58 wherein T is 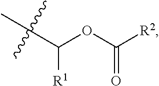
R¹ is H, R² is phenyl and G is H.

95. The compound of claim 58 wherein T is (CH₂)ₙCF₃, n is 2, G is (CH₂)ₙCF₃ and n is 2.

96. The compound of claim 58 wherein T is 2-morpholinoethyl and G is ethyl.

97. The compound of claim 58 wherein T is 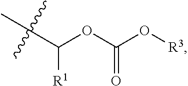
R¹ is methyl, R³ is ethyl and G is ethyl.

98. The compound of claim 58 wherein T is 2-morpholinoethyl and G is 2-morpholinoethyl.

99. The compound of claim 58 wherein T is benzyl and G is 2-morpholinoethyl.

100. The compound of claim 58 wherein T is indanyl and G is 2-morpholinoethyl.

101. The compound of claim 58 wherein T is 2-morpholinoethyl, G is (CH₂)ₙCF₃ and n is 2.

102. The compound of claim 58 wherein T is 2-morpholinoethyl and G is isoamyl.

103. The compound of claim 58 wherein T is (CH₂)ₙCF₃, n is 1, G is (CH₂)ₙCF₃ and n is 1.

104. A pharmaceutical formulation comprising the compound of claim 54 and a pharmaceutically acceptable excipient.

105. The pharmaceutical formulation of claim 104 wherein the formulation is adapted for inhalation.
